(19) United States
(12) Patent Application Publication
(10) Pub. No.: US 2023/0190662 A1
(43) Pub. Date: Jun. 22, 2023

(54) Efficacy of a Gastro-Retentive Bile Acid Sequestrant Dosage Form

(71) Applicant: Ironwood Pharmaceuticals, Inc., Boston, MA (US)

(72) Inventors: Mark G. Currie, Boston, MA (US); Ahmad Hashash, Southborough, MA (US); Bernard Joseph Lavins, Lexington, MA (US); Michael Hall, Weston, MA (US); Silva Karasik, Billerica, MA (US)

(21) Appl. No.: 16/631,208

(22) PCT Filed: Jul. 19, 2018
(86) PCT No.: PCT/US2018/042904

§ 371 Date: Jan. 15, 2020

Related U.S. Application Data
(60) Provisional application No. 62/534,591, filed on Jul. 19, 2017.
(60) Provisional application No. 62/681,633, filed on Jun. 6, 2018.

(51) Int. Cl. A61K 9/28; A61K 31/785; A61K 9/20; A61K 45/06; A61P 1/04

(52) U.S. Cl. CPC A61K 9/284; A61K 31/785; A61K 9/2054; A61K 9/2013; A61K 9/2009; A61K 45/06; A61P 1/04

(57) ABSTRACT

Methods and formulations of reducing one or more symptoms of gastroesophageal reflux disease (GERD) in a human patient with symptomatic GERD not completely responsive to proton pump inhibitors (PPIs). The patient is administered a therapeutically effective amount of an enteric coated gastro-retentive oral dosage form in the form of a tablet of a bile acid sequestrant dispersed in a polymeric matrix consisting essentially of poly(alkylene)oxide and one or more filler or compressing agent such that the patient experiences a clinically meaningful reduction in one or more symptoms of GERD.

### TECHNICAL FIELD

This disclosure relates, inter alia, to methods of using gastro-retentive oral dosage forms comprising a bile acid sequestrant for treating, for example, GERD.

### BACKGROUND

Bile reflux occurs when bile flows upward (refluxes) from the small intestine into the stomach and then into the esophagus. Bile acid refluxes into the esophagus when the lower esophageal sphincter (LES), separating the esophagus and stomach, malfunctions.

Many disorders and/or symptoms are associated with bile reflux, either alone or in combination with acid reflux, including, inter alia, gastroesophageal reflux disease, or GERD, heartburn, indigestion, dyspepsia, erosive esophagitis, peptic ulcer, gastric ulcer, esophageal ulcers, esophagitis, laryngitis, pharyngitis, coarse or hoarse voice, GERD-related pulmonary dysfunction (such as coughing and/or asthma), Barrett's esophagus, esophageal cancer (e.g., adenocarcinoma), and gastritis.

GERD is a generic term encompassing diseases with various digestive symptoms, such as pyrosis, acid regurgitation, obstructed admiration, aphagia, pectoralgia, permeating feeling and the like sensibility caused by reflux in the esophagus and stagnation of gastric contents, duodenal juice, pancreatic juice and the like. The term covers both reflux esophagitis in which erosion and ulcers are endoscopically observed, and esophageal regurgitation-type non-ulcer dyspepsia (NUD), in which no abnormality is endoscopically observed. GERD occurs when the LES does not close properly and stomach contents leak back, or reflux, into the esophagus.

Presently, the main therapies employed in the treatment of GERD and upper GI tract disorders include agents for reducing the stomach acidity, such by using the histamine H2-receptor antagonists or proton pump inhibitors (PPIs). H2 blockers are drugs that inhibit the production of acid in the stomach. PPIs act by inhibiting the parietal cell H⁺/K⁺ ATPase proton pumps responsible for acid secretion from these cells. PPIs, such as omeprazole and its pharmaceutically acceptable salts, are disclosed, for example, in EP 05129, EP 124495 and U.S. Pat. No. 4,255,431. Despite some efficacy, PPIs have notable limitations. Some GERD patients are not fully responsive to treatment with PPI.

### SUMMARY

This disclosure provides a method of reducing one or more symptoms of gastroesophageal reflux disease (GERD) in a human patient with symptomatic GERD not completely responsive to proton pump inhibitors (PPIs), comprising administering to the patient a therapeutically effective amount of an enteric coated gastro-retentive oral dosage form in the form of a tablet of a bile acid sequestrant dispersed in a polymeric matrix comprising of or consisting essentially of poly(alkylene)oxide and, in certain embodiments, one or more filler or compressing agent selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate. The tablet is coated with an enteric coating for prolonged retention of the bile acid sequestrant in the stomach of the patient (and in certain embodiments, in a dose of 500 mg, 700 mg, 750 mg, 1,000 mg, 1,400 mg, 1,500 mg, 2,100 mg, or more; in certain further embodiments, twice per day), and administering a pharmaceutical composition comprising a PPI; wherein the patient experiences a clinically meaningful reduction in one or more symptoms of GERD.

In other aspects, this disclosure provides an enteric coated gastro-retentive oral dosage form in the form of a tablet comprising: colesevelam or colesevelam hydrochloride dispersed in a polymeric matrix comprising of or consisting essentially of poly(alkylene)oxide and, in certain embodiments, one or more filler or compressing agent selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate. The tablet is coated with an enteric coating for prolonged retention of the bile acid sequestrant in the stomach.

[E01] According to a first aspect of the invention, there is provided, a method of reducing one or more symptoms of gastroesophageal reflux disease (GERD) in a human patient with symptomatic GERD not completely responsive to proton pump inhibitors (PPIs), comprising administering to the patient a therapeutically effective amount of an enteric coated gastro-retentive oral dosage form in the form of a tablet of a bile acid sequestrant dispersed in a polymeric matrix consisting essentially of polyethylene oxide CAS Number 25322-68-3, approximate molecular weight 300,000 (PEG-7M) (Polyox™ WSR N-750) and one or more filler or compressing agent selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate, wherein the tablet has a tablet core and is coated with an enteric coating, for prolonged retention of the bile acid sequestrant to the stomach of the patient, and administering a pharmaceutical composition comprising a PPI; wherein the patient experiences a clinically meaningful reduction in one or more symptoms of GERD.

[E02] The method according to E01, wherein the bile acid sequestrant is colesevelam or colesevelam hydrochloride.

[E03] The method according to any one of E01-E02, wherein the bile acid sequestrant is colesevelam hydrochloride.

[E04] The method according to any one of E01-E03, wherein the patient is administered a dose of 500 mg, 700 mg, 750 mg, 1,000 mg, 1400 mg, 1,500 mg, or 2,100 mg, or more, of the bile acid sequestrant, twice per day.

[E05] The method according to any one of E01-E04, wherein the patient is administered a dose of 1500 mg of bile acid sequestrant, twice per day.

[E06] The method according to any one of E01-E05, wherein the dose is 1,500 mg, twice per day.

[E07] The method according to any one of E01-E06, wherein the dose of 1,500 mg is administered as either 2 tablets, each tablet having 750 mg of the bile acid sequestrant or as 3 tablets, each tablet having 500 mg of the bile acid sequestrant, twice per day.

[E08] The method according to any one of E01-E07, wherein the dose of 1,500 mg is administered as 2 tablets, each tablet having 750 mg of the bile acid sequestrant twice per day.

[E09] The method according to any one of E01-E07, wherein the dose of 1,500 mg is administered as 3 tablets, each tablet having 500 mg of the bile acid sequestrant, twice per day.

[E10] The method according to any one of E01-E09, wherein prior to administering said enteric coated gastro-retentive, oral dosage form in the form of a tablet of a bile acid sequestrant, the patient was not completely responsive to other treatments, including individually optimized, standard-labeled dose daily PPI therapy for a minimum of 8 weeks.

[E11] The method according to any one of E01-E10, wherein the patient has erosive esophagitis.

[E12] The method according to E11, wherein the patient has erosive esophagitis on esophagogastroduodenoscopy (EGD) with approximately 48 to 96 hours of pH monitoring with a catheter-free, capsule-based pH monitoring device that is attached to the patient's esophagus.

[E13] The method according to E11 or E12, wherein the patient has evidence of pathological acid reflux on EGD with approximately 48 to 96 hours of pH monitoring with a catheter-free, capsule-based pH monitoring device that is attached to the patient's esophagus.

[E14] The method according to any one of E01-E13, wherein said enteric coated gastro-retentive, oral dosage form in the form of a tablet of a bile acid sequestrant is administration for eight weeks (eight treatment weeks) or more.

[E15] The method according to any one of E01-E14, wherein the patient experiences a clinically meaningful weekly heart burn severity score reduction compared to baseline.

[E16] The method according to any one of E01-E15, wherein the patient experiences a clinically meaningful weekly heart burn severity score reduction compared to baseline of at least 30% for at least four of the eight treatment weeks, including at least one of the last two weeks.

[E17] The method according to any one of E01-E16, wherein the patient experiences a clinically meaningful weekly heart burn severity score reduction compared to baseline of at least 45% for at least four of the eight treatment weeks, including at least one of the last two weeks.

[E18] The method according to any one of E01-E17, wherein the patient experiences a clinically meaningful Weekly Regurgitation Frequency Score (WRFS) reduction compared to baseline.

[E19] The method according to any one of E01-E18, wherein the patient experiences a clinically meaningful a clinically meaningful Weekly Regurgitation Frequency Score (WRFS) reduction compared to baseline of at least 30% for at least four of the eight treatment weeks, including one of the last two weeks.

[E20] The method according to any one of E01-E19, wherein the patient experiences a clinically meaningful a clinically meaningful Weekly Regurgitation Frequency Score (WRFS) reduction compared to baseline of at least 45% for at least four of the eight treatment weeks, including one of the last two weeks.

[E21] The method according to any one of E01-E20, wherein the dosage form is retained in the stomach until it is substantially or completely disintegrated.

[E22] The method according to any one of E01-E21, wherein the enteric coated gastro-retentive, oral dosage form further comprises at least about 0.06% per weight butylated hydroxytoluene of the tablet core.

In another aspect of the invention, there is provided a method of reducing one or more symptoms of gastroesophageal reflux disease (GERD) in a human patient with symptomatic GERD not completely responsive to proton pump inhibitors (PPI), comprising administering a therapeutically effective amount of an enteric coated gastro-retentive oral dosage form in the form of a tablet of colesevelam or colesevelam hydrochloride a dispersed in a polymeric matrix consisting essentially of PEG-7M (polyethylene oxide CAS Number 25322-68-3, approximate molecular weight 300,000 (Polyox™ WSR N-750)), and one or more filler or compressing agent selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate, wherein the tablet has a tablet core and is coated with a polyvinyl alcohol based enteric coating, for prolonged retention of the bile acid sequestrant in the stomach of the patient in a dose of 1,500 mg twice daily; wherein: prior to administering said enteric coated gastro-retentive, oral dosage form in the form of a tablet of a bile acid sequestrant, the patient was not completely responsive to other treatments, including individually optimized, standard-labeled dose daily PPI therapy for a minimum of 8 weeks, the patient has erosive esophagitis; said enteric coated gastro-retentive, oral dosage form in the form of a tablet of a bile acid sequestrant is administration for eight weeks (eight treatment weeks); the dosage form is retained in the stomach until it is substantially or completely disintegrated; the patient experiences a clinically meaningful weekly heart burn severity score reduction compared to baseline; and clinically meaningful Weekly Regurgitation Frequency Score (WRFS) reduction compared to baseline.

[E23] The method according to E23, wherein the patient experiences a clinically meaningful weekly heart burn severity score reduction compared to baseline of at least 30% for at least four of the eight treatment weeks, including at least one of the last two weeks.

[E24] The method according to any one of E23 or E24, wherein the patient experiences a clinically meaningful weekly heart burn severity score reduction compared to baseline of at least 45% for at least four of the eight treatment weeks, including at least one of the last two weeks.

[E25] The method according to any one of E23-E25, wherein the patient experiences a clinically meaningful Weekly Regurgitation Frequency Score (WRFS) reduction compared to baseline of at least 30% for at least four of the eight treatment weeks, including at least one of the last two weeks.

[E26] The method according to any one of E23-E26, wherein the patient experiences a clinically meaningful Weekly Regurgitation Frequency Score (WRFS) reduction compared to baseline of at least 45% for at least four of the eight treatment weeks, including at least one of the last two weeks. The method according to any one of E23-E27, wherein the enteric coated gastro-retentive, oral dosage form further comprises at least about 0.06% per weight of butylated hydroxytoluene of the tablet core.

[E27] In another aspect of the invention, an enteric coated gastro-retentive oral dosage form in the form of a tablet is provided. The dosage form comprises: colesevelam or colesevelam hydrochloride dispersed in a polymeric matrix consisting essentially of PEG-7M (polyethylene oxide CAS Number 25322-68-3, approximate molecular weight 300,000 (Polyox™ WSR N-750)) and one or more filler or compressing agent selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate, wherein the tablet has a tablet core and is coated with an enteric coating, for prolonged retention of the bile acid sequestrant in the stomach.

[E28] The dosage form of E28, wherein the dosage form is for prolonged retention in the stomach until it is substantially or completely disintegrated.

[E29] The dosage form of E28 or E29, wherein the one or more filler or compressing agent is microcrystalline cellulose at about 1-10% w/w of the tablet core, butylated hydroxytoluene at about 0.01 to 0.10% w/w of the tablet core, colloidal silicon dioxide at about 1-5% w/w of the tablet core, magnesium stearate at about 0.1 to 1.0% w/w of the tablet core.

[E30] The dosage form of any one of E28 to E30, wherein the enteric coating is a polyvinyl alcohol-based enteric coating.

[E31] The dosage form of E31, wherein the enteric coating is a polyvinyl alcohol based enteric coating.

[E32] The dosage form of any one of E28 to E32, wherein the enteric coating is a polyvinyl alcohol based enteric coating at about 1-5% w/w of the tablet core.

[E33] The dosage form of any one of E28 to E33, wherein the PEG-7M is at about 30 to about 60% w/w of the tablet core.

[E34] The dosage form of any one of E28 to E34, wherein the PEG-7M is about 46% w/w of the tablet core.

[E35] The dosage form of any one of E28 to E35, wherein the enteric coating is a polyvinyl alcohol based enteric coating at 3% w/w of the tablet core.

[E36] The dosage form of any one of E28 to E36, wherein the one or more filler or compressing agent is microcrystalline cellulose at about 5.4% w/w of the tablet core, butylated hydroxytoluene at least about 0.06 w/w of the tablet core, colloidal silicon dioxide at about 2.0% w/w of the tablet core, magnesium stearate at about 0.5% w/w of the tablet core.

The dosage form of any one of E26 to E35, wherein the enteric coated gastro-retentive, oral dosage form further comprise at least about 0.06% butylated hydroxytoluene per weight of the tablet core.

[E37] In yet another aspect of the invention, a pharmaceutical composition is provided. The pharmaceutical comprises the gastro-retentive oral dosage form of any of E28-E37.

[E38] The pharmaceutical composition of E38, further comprising an additional therapeutic agent.

[E39] In still another aspect of the invention, there is provided a method of treating a disease selected from heartburn, indigestion, dyspepsia, erosive esophagitis, peptic ulcer, gastric ulcer, esophageal ulcers, esophagitis, laryngitis, pharyngitis, coarse voice, gastroesophageal reflux disease (GERD), Barrett's esophagus, gastric cancer, esophageal cancer (e.g., adenocarcinoma), gastritis and GERD-related pulmonary dysfunction, and symptomatic GERD not completely responsive to proton pump inhibitor, comprising administering a therapeutically effective amount of a gastro-retentive, oral dosage form of any of E28-E38 or the pharmaceutical composition of E39 or E40 to a subject in need thereof.

[E40] The method of E40, wherein the disease is gastroesophageal reflux disease (GERD).

[E41] The method of E40, wherein the disease is symptomatic GERD not completely responsive to proton pump inhibitor.

[E42] The method of E40, wherein the disease is symptomatic GERD not completely responsive to proton pump inhibitor and the method comprises administering a therapeutically effective amount of the pharmaceutical composition of E38.

[E43] In another aspect of the invention, there is provided a method to treat/prevent signs and/or symptoms associated with bile acid reflux comprising administering to the patient a therapeutically effective amount of an enteric coated gastro-retentive oral dosage form in the form of a tablet of a bile acid sequestrant dispersed in a polymeric matrix consisting essentially of poly(alkylene)oxide and one or more filler or compressing agent selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate, wherein the tablet is coated with an enteric coating, for prolonged retention of the bile acid sequestrant to the stomach of the patient, to a patient in an amount effective to ameliorate, reduce, palliate, lessen, delay, and/or alleviate one or more of the signs and/or symptoms associated with bile acid reflux.

[E44] The method of E44, wherein the bile acid sequestrant is colesevelam or colesevelam hydrochloride.

[E45] The method of E44 or E45, wherein the dosage form is retained in the stomach until it is substantially or completely disintegrated.

### BRIEF DESCRIPTION OF THE DRAWINGS

FIG. 1 shows a schematic of an efficacy study design (see Example 1).

FIG. 2-FIG. 4 show results of % change from baseline in WHSS at week 8 (mITT population). *Nominal P=0.04. LOCF=last observation carried forward (FIG. 2) (N range=68-71); MMRM=mixed model repeated measures (FIG. 3) (N range=61-66); OC=observed cases (FIG. 4) (N range=61-66). Weekly Heartburn Severity Score (WHSS) is defined as the weekly average of the Daily Heartburn Severity Score (DHSS). DHSS is defined as the maximum of the 3 items measuring heartburn from a particular day, as collected with the mRESQ-eD. * P-values are based on a pairwise comparison versus placebo in an ANCOVA model with fixed effect terms for treatment group and esophagitis and baseline value as covariate. Trend test nominal P=0.0225, performed using linear contrast statement. PBO=placebo.

FIG. 5 (LOCF)-FIG. 6 (MMRM) show results of weekly % change from baseline in WHSS at week 8. Week 8 difference—1500 mg IW-3718 vs. placebo is 11.9% for LOCF and 9.4% for MMRM. LOCF=last observation carried forward; MMRM=mixed model repeated measures Weekly Heartburn Severity Score (WHSS) is defined as the weekly average of the Daily Heartburn Severity Score (DHSS). DHSS is defined as the maximum of the 3 items measuring heartburn from a particular day, as collected with the mRESQ-eD.

FIG. 7 shows results of weekly % change in WHSS of EE patients (N range=36-37) (mITT population; MMRM). Week 8 difference—1500 mg IW-3718 vs, placebo is 12.2%.

FIG. 8 shows results of weekly % change in WHSS in patients with baseline greater than or equal to 2.5 (N range=36-37) (mITT population; MMRM). Week 8 difference— 1500 mg IW-3718 vs, placebo (PBO or Pbo, or the like) is 12.7%.

FIG. 9 shows results of % overall heartburn responders (mITT population). An Overall Heartburn Responder had a decrease of at least 30% (45%) in WHSS for at least 4 of the 8 treatment weeks, including at least 1 of the last 2 weeks.

FIG. 10 shows results of % overall heartburn responders (mITT population) for EE patients. EE=Erosive Esophagitis Patients. An Overall Heartburn Responder had a decrease of at least 30% (45%) in WHSS for at least 4 of the 8 treatment weeks, including at least 1 of the last 2 weeks.

FIG. 11 and FIG. 13 show results of % change in WRFS, baseline greater than zero, at week 8 (mITT population; MMRM). The difference between 1500 mg and PBO was 16.6% (FIG. 11) or 16.7% (FIG. 13).

FIG. 12 and FIG. 14 show results of % change in WRFS, baseline greater than or equal to 2 (mITT population; MMRM). The difference between 1500 mg and PBO was 7.0%. EE=Erosive Esophagitis Patients. An Overall Heartburn Responder had a decrease of at least 30% (45%) in WHSS for at least 4 of the 8 treatment weeks, including at least 1 of the last 2 weeks.

FIG. 15 shows results of weekly % change in WRFS, BL greater than 0, at week 8 (mITT population; MMRM) for EE patients. MMRM=mixed model repeated measures. Weekly Regurgitation Frequency Score (WRFS) is defined as the weekly average of the Daily Regurgitation Frequency Score (DRFS). DRFS is defined as the maximum of the 2 items measuring regurgitation from a particular day, as collected with the mRESQ-eD. The difference between 1500 mg and PBO was 23.6%.

FIG. 16 shows results of % overall regurgitation responders—BL>0 (mITT Population). An Overall Regurgitation Responder had a decrease of at least 30% (45%) in WRFS for 4 of the 8 treatment weeks, including 1 of the last 2 weeks.

FIG. 17 shows results of % overall regurgitation responders—BL>0 (mITT Population) in EE patients.

FIG. 18 shows results of % degree of relief responders with baseline greater than or equal to 2 (mITT Population).

FIG. 19 shows results of % degree of relief responders (HB, RG, GERD)(mITT population). GERD=Gastroesophageal Reflux Disease Patients; HB=Heartburn; RG=Regurgitation. A % Degree of Relief Responder was “significantly” or “moderately” relieved for 4 of the 8 treatment weeks.

FIG. 20 shows results of proportion of days with nighttime awakening during treatment period (mITT population). \hat{ }Nominal p=0.0241, obtained from a pairwise comparison versus placebo in an ANCOVA model with fixed-effect terms for treatment group and esophagitis status and baseline values as a covariate

FIG. 21 shows summary of mRESQ-eD validation results. BL (baseline); W8 (week 8). Leftmost reference line is median responder percent change score (−0.65); right reference line is median responder percent change score (−0.44).

FIG. 22 shows a schematic of a scintigraphy study design. * Prior to study dosing, the subject is randomized to one of six breakfast sequences (see Example 2). Note that there is no day 0.

FIG. 23-FIG. 26 shows scintigraphy study results:

FIG. 23 shows IW-3718 took longer to disintegrate in the stomach than IR colesvelam. 16 of 18 subjects showed IW-3718 tablet retained in the stomach until complete disintegration. Gastroretentive (per literature, label claims): Dosage forms designed to be retained in the upper gastrointestinal tract for a prolonged period of time, during which they release the drug on a controlled basis.

Prolonged period of time (per literature): Generally accepted as >4 h in fed state. 4 h (range of 2-6 h) represents the gastric emptying of non-digestible solids in fed state.

FIG. 24 shows IW-3718 took longer to disintegrate in the stomach than IR colesvelam. All of the subjects' data are graphed.

FIG. 25 shows radioactivity from IW-3718 takes longer to empty from gastric cavity than from immediate release dosage form.

FIG. 26 shows radioactivity from IW-3718 takes longer to empty from gastric cavity than from immediate release dosage form.

FIG. 27 shows complete gastric emptying for IW-3718 is later than the immediate release dosage form.

FIG. 28 shows that formulation 2b is more stable than formulation 2a.

FIG. 29 shows stability data for formulations 2a and 2b. Again, formulation 2b is more stable than formulation 2a.

FIG. 30 shows importance of BHT for stability. FIG. 30a shows comparative drug release data; FIG. 30b shows primera forced PEO degradation study data.

“LS” stands for least squares.

### DETAILED DESCRIPTION

As used herein, the word “a” or “plurality” before a noun represents one or more of the particular noun.

As used herein, the term “subject” and “patient” are used interchangeably. A patient or a subject is a human patient or a human subject.

The term “PEG-7M” used herein refers to polyethylene oxide CAS Number 25322-68-3, approximate molecular weight 300,000 (PEG-7M) (Polyox™ WSR N-750). The terms “Polyox™ WSR N-750” and “PEG-7M,” both refer to polyethylene oxide CAS Number 25322-68-3, approximate molecular weight 300,000.

As used herein, “effective treatment” refers to treatment producing a beneficial effect, e.g., amelioration of at least one symptom of a disease or disorder in a patient.

The term “effective amount” or “a therapeutically effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease in a patient, or any other desired alteration of a biological system. An effective amount can be administered in one or more administrations.

The term “gastro-retentive dosage form” denotes dosage forms which are designed to be retained in the upper gastrointestinal tract for a prolonged period of time (generally, at least 4 hours) during which they release the drug on a controlled basis.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Disorders and/or Symptoms Associated with Bile Reflux

The upper gastrointestinal tract consists of the mouth, a portion of the throat, the esophagus, the stomach and the duodenum, and the uppermost part of the small intestine. The esophagus carries food, liquids, and saliva from the mouth to the stomach by coordinated contractions of its muscular lining. This process is automatic. The muscular layers of the esophagus are normally pinched together at both the upper and lower ends by muscles called sphincters. When one swallows, the sphincters relax automatically to allow food or drink to pass from the mouth into the stomach. The muscles then close rapidly to prevent the swallowed food or drink from leaking out of the stomach back into the esophagus or into the mouth. These sphincters make it possible to swallow while lying down or even upside-down.

Bile reflux occurs when bile, a digestive fluid produced in the liver, flows upward (refluxes) from the small intestine into the stomach and then into the esophagus. Bile reflux often accompanies acid reflux, and together they may cause inflammation of the esophageal lining and potentially increased risk of esophageal cancer. See AJG (1999) 94(12):3649-3650. Bile reflux may also affect the stomach, causing inflammation (gastritis, which, if untreated, can lead to peptic ulcers). Bile reflux can be difficult to distinguish from acid reflux because the signs and symptoms are similar, and the two conditions frequently occur at the same time. Unlike acid reflux, bile reflux inflames the stomach, often causing a gnawing or burning pain in the upper abdomen. Other signs and symptoms may include: frequent heartburn, i.e., a burning sensation in the chest that sometimes spreads to the throat along with a sour taste in the mouth; nausea; vomiting bile; a cough; or hoarseness.

Bile acids are steroid acids found predominantly in the bile of mammals. They are produced in the liver by the oxidation of cholesterol and they and are stored in gallbladder and secreted into the intestine in the form of salts. They act as surfactants, emulsifying lipids and assisting with the absorption and digestion of dietary fat and cholesterol.

The principal bile acids are: cholic acid, chenodeoxycholic acid, deoxycholic acid, taurocholic acid, and glycocholic acid. The chemical distinctions between different bile acids are small, depending only on the presence or absence of hydroxyl groups on positions 3, 7, and 12. In humans, the most prevalent bile acids are cholic acid and chenodeoxycholic acid, and their conjugates with taurine and glycine (glycocholate and taurocholate). Some mammals synthesize predominantly deoxycholic acid.

The body synthesizes about 800 mg of cholesterol per day and about half of that is used for bile acid synthesis. In total about 20-30 grams of bile acids are secreted into the intestine daily; about 90% of excreted bile acids are reabsorbed (by active transport in the ileum) and recycled. Since bile acids are made from endogenous cholesterol, the enterohepatic circulation of bile acids may be disrupted as a way to lower cholesterol. This is the usual therapeutic rationale for administering bile acid sequestrants.

Though bile acids play an important role in the digestive process, the prolonged presence or excess of bile acids in the stomach and esophagus can result in toxic effects on regional tissues. Duodenogastroesophageal reflux (DGER), which contains bile acids, is thought to produce symptoms such as retrosternal pain, heartburn, nausea, and vomiting, and is associated with more severe esophageal pathology in patients with gastroesophageal reflux disease (GERD) and Barrett's esophagus, a precancerous change in the esophagus.

Bile reflux and acid reflux can seriously damage esophageal tissue, as the esophagus, unlike the stomach, lacks a sticky mucous coating to protect it from the corrosive effects of stomach acids. And although bile reflux can injure the esophagus on its own—even when the pH of the reflux is neutral or alkaline—the combination of bile and acid reflux seems to be particularly harmful, increasing the risk of complications.

Additional symptoms are produced when bile acids reflux into the throat. The throat is close to the esophagus, separated only by the epiglottis, a flap which separates the esophagus from the trachea (the windpipe) and prevents inhalation of food or drink into the lungs.

Disorders and/or symptoms that are believed to be associated with bile reflux, either alone or in combination with acid reflux, include, for instance, heartburn, indigestion, dyspepsia, erosive esophagitis, peptic ulcer, gastric ulcer, esophageal ulcers, esophagitis, laryngitis, pharyngitis, coarse or hoarse voice, and GERD-related pulmonary dysfunction such as coughing and/or asthma. Further complications that are believed to occur as a result of chronic bile reflux are, for instance, gastroesophageal reflux disease, or GERD; Barrett's esophagus; esophageal cancer (e.g., adenocarcinoma) and gastritis.

GERD is a generic term encompassing diseases with various digestive symptoms such as pyrosis, acid regurgitation, obstructed admiration, aphagia, pectoralgia, permeating feeling and the like sensibility caused by reflux in the esophagus and stagnation of gastric contents, duodenal juice, pancreatic juice and the like. The term covers both reflux esophagitis in which erosion and ulcers are endoscopically observed, and esophageal regurgitation-type non-ulcer dyspepsia (NUD) in which no abnormality is endoscopically observed. GERD occurs when the LES does not close properly and stomach contents leak back, or reflux, into the esophagus. A persistent GERD patient is a patient who does not respond to PPI.

A hiatal hernia may contribute to causing GERD and can happen in people of any age. Other factors that may contribute to GERD include, but are not limited to, alcohol use, being overweight, pregnancy, smoking, Zollinger-Ellison syndrome, hypercalcemia, and scleroderma. Also, certain foods can be associated with reflux events, including, citrus fruits, chocolate, drinks with caffeine, fatty and fried foods, garlic and onions, mint flavorings, spicy foods, and tomato-based foods, like spaghetti sauce, chili, and pizza.

The inner mucosa of the esophagus is lined with non-keratinized stratified squamous epithelium arranged in longitudinal folds. Damage to the lining of the esophagus causes the normal squamous cells lining the esophagus to turn into a type of cell not usually found in humans, called specialized columnar cells. That conversion of cells in the esophagus by the acid reflux is known as Barrett's Esophagus. Although people who do not have heartburn can have Barrett's esophagus, it is found about three to five times more often in people with this condition. Barrett's esophagus does not cause symptoms itself and is important only because it seems to precede the development of a particular kind of cancer—esophageal adenocarcinoma. The risk of developing adenocarcinoma is 30 to 125 times higher in people who have Barrett's esophagus than in people who do not. This type of cancer is increasing rapidly in white men. This increase may be related to the rise in obesity and GERD.

Barrett's esophagus has no cure, short of surgical removal of the esophagus, which is a serious operation. Surgery is recommended only for people who have a high risk of developing cancer or who already have it. Most physicians recommend treating GERD with acid-blocking drugs, since this is sometimes associated with improvement in the extent of the Barrett's tissue. However, this approach has not been proven to reduce the risk of cancer. Treating reflux with a surgical procedure for GERD also does not seem to cure Barrett's esophagus. Several different experimental approaches are under study. One attempts to see whether destroying the Barrett's tissue by heat or other means through an endoscope can eliminate the condition. This approach, however, has potential risks and unknown effectiveness.

Esophageal cancer can occur almost anywhere along the length of the esophagus, but it frequently starts in the glandular cells closest to the stomach (adenocarcinoma). Because esophageal cancer may not be diagnosed until it's quite advanced, the outlook for people with the disease is often poor. The risk of cancer of the esophagus is increased by long-term irritation of the esophagus, such as with smoking, heavy alcohol intake, and Barrett's esophagitis. Thus, there is a link between esophageal cancer and bile reflux and acid reflux. In animal models, bile reflux alone has been shown to cause cancer of the esophagus.

Unlike acid reflux, bile reflux usually cannot be controlled by changes in diet or lifestyle. Instead, bile reflux is most often managed with certain medications or, in severe cases, with surgery. Neither solution is uniformly effective, and some people continue to experience bile reflux even after treatment.

Numerous medications are used to treat heartburn and indigestion. Presently, the main therapies employed in the treatment of GERD and upper GI tract disorders include agents for reducing the stomach acidity, such as by using the histamine H2-receptor antagonists or proton pump inhibitors (PPIs). H2 blockers are drugs that inhibit the production of acid in the stomach. Exemplary histamine H2-receptor antagonists include, for example, cimetidine (as sold under the brand-name TAGAMET HB®), famotidine (as sold under the brand-name PEPCID AC®), nizatidine (as sold under the brand-name AXID AR®), and ranitidine (as sold under the brand-name ZANTAC 75®). Both types of medication are effective in treating heartburn caused by acid reflux and usually eliminate symptoms within a short period of time.

PPIs act by inhibiting the parietal cell H⁺/K⁺ ATPase proton pumps responsible for acid secretion from these cells. PPIs, such as omeprazole and its pharmaceutically acceptable salts are disclosed, for example, in EP 05129, EP 124495 and U.S. Pat. No. 4,255,431. Despite their well-documented efficacy, PPIs have notable limitations. For example, patients who are non-responsive to treatment with PPI inhibitor alone may be non-responsive because even though the PPI is decreasing acid reflux from the stomach, bile acid from the duodenum is still present. Also, some patients with GERD are not fully responsive.

GERD is a chronic and common medical disorder with a prevalence estimated at approximately 20% to 40% in Western countries and is associated with rising healthcare utilization and cost. Currently, proton pump inhibitors (PPIs) are the standard of care for GERD; however, it is estimated that approximately 10% to 40% of GERD patients remain symptomatic on standard-dose PPI therapy. DGER is hypothesized to be a potential cause of incomplete symptom response in patients who continue to experience bothersome GERD symptoms despite treatment with PPIs.

Bile Acid Sequestrants

Bile acid sequestrants include, for example, cholestyramine (i.e., QUESTRAN®, QUESTRAN LIGHT®, CHOLYBAR®, CA registry no. 11041-12-6), colesevelam (i.e., WELCHOL®, CA registry nos. 182815-43-6 and 182815-44-7), Selevamer (Rinogel®) and colestipol (i.e., COLESTID®, CA registry nos. 50925-79-6 and 37296-80-3), or any of their pharmaceutically acceptable salts or mixtures thereof.

Colesevelam or colesvelam HCl (may be referred to herein jointly as colesevelam) is an orally administered, nonabsorbed, nondigestible polymer that binds bile acids in the GI tract. Colesevelam was approved in 2000 in the United States (US) as the active ingredient in Welchol™ and is indicated as an adjunct to diet and exercise for reduction of elevated low-density lipoprotein cholesterol (LDL-C) in adults with primary hyperlipidemia. Colesevelam is currently available as an immediate-release formulation only. Colesevelam is not systemically absorbed and does not interfere with systemic drug metabolizing enzymes. Distribution of colesevelam is limited to the GI tract and elimination occurs through fecal excretion.

Gamma Scintigraphy

The technique of gamma scintigraphy was first used to investigate the in vivo release properties of drug formulations in 1976 and since then has become an increasingly useful tool in evaluating the GI performance of pharmaceutical dosage forms. Wilding I R et al. Adv Drug Del Rev 2001; 46: 103-124.

Gamma scintigraphy has been used in the development and evaluation of pharmaceutical drug delivery systems, including enteric-coated tablets and complex controlled-release formulations. Wilding I R. J Clin Pharm 2002; 42: 1200-1210. Dobson C L et al. Int J Pharm 2002; 248: 61-70. Cole E T al., Int J Pharm 2002; 231: 83-95. The technique provides information on the deposition, dispersion and movement of a formulation. Typically such scintigraphic imaging is combined with assay of drug concentrations in blood or urine to provide information concerning the sites of release and of absorption (termed pharmacoscintigraphy) within the body. Connor A L et al. Eur J Pharm Sci 2001; 13: 369-374. As colesevelam is not absorbed, there is no need to take blood samples to assay drug concentrations.

Commonly used radionucleotides for gamma scintigraphic studies include technetium-99m (⁹⁹mTc) and indium-111 (¹¹¹In), both of which are short-lived radionucleotides and used clinically in nuclear medicine and research. In example 2 below, ⁹⁹mTc and ¹¹¹In are used to label immediate release colesevelam and IW-3718 (detailed herein), respectively, to allow the in vivo performance of the formulations to be assessed via scintigraphic imaging.

While the gamma scintigraphic technique is not designed to provide anatomical detail (particularly of the small intestine), the images do allow for identification of the dosage form in the stomach and its subsequent gastric emptying as well as definition of its arrival at the caecum. Therefore, the measurement of various GI transit times for the dosage form is possible. Prior studies have shown that gastric emptying times for normal subjects range from 40 minutes for a liquid meal to 85 minutes for a solid meal. In healthy young adult males it was found that capsules and tablets pass through the colon on average in 20 to 30 hours after ingestion. Total GI transit times are variable and influenced by the ingestion of food, by diet, and by disease, and can range from 1 to more than 60 hours.

Methods

In certain aspects, this disclosure provides methods of reducing one or more symptoms of gastroesophageal reflux disease (GERD) in a human patient with symptomatic GERD not completely responsive to proton pump inhibitors (PPIs). The method comprises administering to the patient a therapeutically effective amount of an enteric coated gastro-retentive oral dosage form in the form of a tablet of a bile acid sequestrant dispersed in a polymeric matrix, which in certain embodiments consists essentially of poly(alkylene)oxide, and, in certain embodiments, one or more filler or compressing agent, which may be selected from one or more of microcrystalline cellulose, butylated hydroxytoluene, colloidal silicon dioxide, lactose, starch, maltodextrins, magnesium stearate, diacetylated monoglycerides, hypromellose, and dibasic calcium phosphate, wherein the tablet has a tablet core and is coated with an enteric coating, which in certain embodiments is a polyvinyl alcohol based enteric coating (such as Opadry® II 85F), for prolonged retention of the bile acid sequestrant in the stomach of the patient (and in certain embodiments, in a dose of the bile acid sequestrant at 500 mg, 700 mg, 750 mg, 1,000 mg, 1,400 mg, 1,500 mg, 2,000 mg, 2,100 mg, or more; in certain further embodiments, twice per day); and in certain embodiments the method further comprises administering a pharmaceutical composition comprising a PPI; wherein in certain embodiments the patient experiences a clinically meaningful reduction in one or more symptoms of GERD.

In certain embodiments, the bile acid sequestrant is administered to a patient at 500 mg, 700 mg, 750 mg, 1,000 mg, 1,400 mg, 1,500 mg, 2,000 mg, 2,100 mg, or more. In some embodiments, the bile acid sequestrant is administered to a patient, one dose per day, two dose per day, or 3 dose per day. In certain embodiments, the bile acid sequestrant is administered to a patient as three 500 mg tablets twice per day.

In another aspect, this disclosure provides a method of reducing one or more symptoms of gastroesophageal reflux disease (GERD) in a human patient with symptomatic GERD not completely responsive to proton pump inhibitors (PPI), comprising administering a therapeutically effective amount of an enteric coated gastro-retentive oral dosage form in the form of a tablet of colesevelam or colesevelam hydrochloride a dispersed in a polymeric matrix comprising of or consisting essentially of polyethylene oxide CAS Number 25322-68-3, approximate molecular weight 300,000 Polyox™ WSR N-750 (INCI name is PEG-7M), and, in certain embodiments, one or more filler or compressing agent selected from microcrystalline cellulose, lactose, starch, maltodextrins and dibasic calcium phosphate, wherein the tablet has a tablet core and is coated with a polyvinyl alcohol based enteric coating, for prolonged retention of the bile acid sequestrant in the stomach of the patient in a dose of 1,500 mg twice daily; wherein: prior to administering said enteric coated gastro-retentive, oral dosage form in the form of a tablet of a bile acid sequestrant, the patient was not completely responsive to other treatments, including individually optimized, standard-labeled dose daily PPI therapy for a minimum of 8 weeks, the patient has erosive esophagitis; said enteric coated gastro-retentive, oral dosage form in the form of a tablet of a bile acid sequestrant is administered for eight weeks (eight treatment weeks) or more; and the dosage form is retained in the stomach until it is substantially or completely disintegrated.

In certain aspects, the methods described herein comprise administering an enteric coated gastro-retentive oral dosage form in the form of a tablet of a bile acid sequestrant to a subject (patient) in an amount effective to ameliorate, reduce, palliate, lessen, delay, and/or alleviate one or more of the signs and/or symptoms associated with bile acid reflux. Such an effective amount may be measured after a single dose, or more commonly after at least one week, more typically after at least two weeks of therapy.

In certain embodiments, the subject is administered the enteric coated gastro-retentive oral dosage form in the form of a tablet of a bile acid sequestrant (also referred to herein as composition, formulation, dosage form, tablet, and the like) in an amount effective to reduce the weekly heartburn severity score (WHSS) by at least 30%. In other embodiments, the subject is administered the composition in an amount effective to reduce the WHSS by at least 45%, for example, at least 50%, at least 55%, at least 60%, at least 65% or more.

In other embodiments, the subject is administered the enteric coated gastro-retentive oral dosage form in the form of a tablet of a bile acid sequestrant in an amount effective to reduce the Weekly Regurgitation Frequency Score (WRFS) by at least 30%. In other embodiments, the subject is administered with the composition in an amount effective to reduce the WHSS by at least 45%, for example, at least 50%, at least 55%, at least 60%, at least 65% or more.

In still other embodiments, the subject is administered the enteric coated gastro-retentive oral dosage form in the form of a tablet of a bile acid sequestrant in an amount effective to reduce the saliva bile acid levels by at least 10%, when measured more than 2 hours after a meal. In other embodiments, the subject is administered with the composition in an amount effective to reduce the saliva total bile acid levels by at least 15%, for example, at least 20%, at least 30%, at least 40%, at least 50% or more, when compared with levels prior to administration of said composition when measured more than 2 hours after a meal.

In still other embodiments, the subject is administered the enteric coated gastro-retentive oral dosage form in the form of a tablet of a bile acid sequestrant in an amount effective to reduce the saliva bile acid levels to 200 μmol/L or below when measured more than 2 hours after a meal. In other embodiments, the subject is administered with the composition in an amount effective to reduce the saliva total bile acid levels to 150 μmol/L or below, 100 μmol/L or below, 75 μmol/L or below, 50 μmol/L or below, or lower, when measured more than 2 hours after a meal.

In certain embodiments, the bile acid sequestrant is colesevelam or colesevelam hydrochloride. The colesevelam or colesevelam hydrochloride may be obtained from suitable sources and may be DSM or Formosa.

In certain embodiments, each dose of the enteric coated gastro-retentive oral dosage form in the form of a tablet for prolonged retention of the bile acid sequestrant in the stomach of the patient is in a dose of 500 mg, 700 mg, 750 mg, 1,000 mg, 1,400 mg, 1,500 mg, 2,000 mg, 2,100 mg, or more. In certain further embodiments, the dose is administered twice per day. A dose may be several dosage forms (tablets) disclosed herein or only one. In certain embodiments, two tablets are administered to the patient twice per day. In other embodiments, three tablets are administered to the patient twice per day.

In certain embodiments, an ingredient of this polymeric matrix is at least one hydrophilic, water-swellable, erodible, or soluble polymer, which may generally be described as an “osmopolymer”, “hydrogel” or “water-swellable” polymer. More than one of such polymers may be combined in a dosage form of the invention to achieve gastric-retention as well as the desired erosion rate.

Polymers suitable for achieving the desired gastro-retentive and sustained-release profiles of the dosage forms used in the methods disclosed herein have the property of swelling as a result of imbibing water from the gastric fluid, and gradually eroding over a time period of several hours. Since erosion of the polymer results from the interaction of the fluid with the surface of the dosage form, erosion initiates more or less simultaneously with the swelling process. While erosion and swelling may occur at the same time, the rate for achieving maximum swelling should be faster than the rate the dosage form fully erodes to achieve the desired release profile. Such polymers may be linear, branched, or cross linked. The polymers may be homopolymers or copolymers.

In some embodiments, the polymer is a polyalkylene oxide. In some embodiments, at least one of the one or more hydrophilic polymers is a polyethylene oxide (PEO). In still other embodiments, the at least one hydrophilic polymer is a polyethylene oxide having a molecular weight of about 300,000 Daltons.

“Polyethylene oxide” or “PEO” refers to a polyethylene oxide polymer that has a wide range of molecular weights. PEO is a linear polymer of unsubstituted ethylene oxide and has a wide range of viscosity-average molecular weights. Non-limiting examples of commercially available PEOs and their approximate molecular weights (in grams/mole or Daltons) are: POLYOX® NF, grade WSR coagulant, approximate molecular weight 5 million; POLYOX® grade WSR 301, approximate molecular weight 4 million; POLYOX® grade WSR 303, approximate molecular weight 7 million; POLYOX® grade WSR N60-K, approximate molecular weight 2 million; POLYOX® grade WSR N-80K, approximate molecular weight 200,000; polyethylene oxide CAS Number 25322-68-3, approximate molecular weight 300,000 Polyox™ WSR N-750 (INCI name: PEG-7M), which is a polymer of ethylene oxide, approximate molecular weight 300,000.

The terms “polyethylene oxide” and “poly(ethylene)oxide” are used interchangeably herein. The terms “polyalkylene oxide” and “poly(alkylene)oxide” are used interchangeably herein.

In some embodiments, the poly(ethylene)oxide is present in the unit dosage form in an amount ranging from 40 weight percent ratio to 75 weight percent ratio of the tablet core. In some embodiments, the poly(ethylene)oxide is present in the unit dosage form in an amount ranging from 40 weight percent ratio to 60 weight percent ratio of the tablet core. In some embodiments, the poly(ethylene)oxide is present in the unit dosage form in an amount ranging from 45 weight percent ratio to 55 weight percent ratio of the tablet core. In some embodiments, the poly(ethylene)oxide is present in the unit dosage form in an amount ranging from 45 weight percent ratio to 60 weight percent ratio of the tablet core. In some embodiments, the poly(ethylene)oxide is present in the unit dosage form in an amount ranging from 40 weight percent ratio to 50 weight percent ratio of the tablet core. In some embodiments, the poly(ethylene)oxide is present in the unit dosage form in an amount ranging from 50 weight percent ratio to 60 weight percent ratio of the tablet core. In some embodiments, the poly(ethylene)oxide is present in the unit dosage form in an amount ranging from 47 weight percent ratio to 53 weight percent ratio of the tablet core.

In certain embodiments, the poly(alkylene)oxide is polyethylene oxide CAS Number 25322-68-3, approximate molecular weight 300,000 (PEG-7M) (Polyox™ WSR N-750). In certain embodiments, the poly(alkylene)oxide is polyethylene oxide CAS Number 25322-68-3, approximate molecular weight 300,000 (PEG-7M)(Polyox™ WSR N-750) at 30 to 46 to 60% w/w of the tablet core weight. The tablets have a core, which in turn is coated to become a coated tablet. In certain embodiments, the poly(alkylene)oxide has approximate molecular weight of 300,000 Daltons. In certain embodiments, the poly(alkylene)oxide yields viscosity of 600 to 1,000 at moderate addition levels.

In other embodiments, the at least one hydrophilic polymers of the dosage form include a cellulose. In certain embodiments, the polymers may be synthetic polymers derived from vinyl, acrylate, methacrylate, urethane, ester and oxide monomers. In other embodiments, they can be derivatives of naturally occurring polymers such as polysaccharides (e.g. chitin, chitosan, dextran and pullulan; gum agar, gum arabic, gum karaya, locust bean gum, gum tragacanth, carrageenans, gum ghatti, guar gum, xanthan gum and scleroglucan), starches (e.g. dextrin and maltodextrin, corn-starch—unmodified or pregelatinized-), hydrophilic colloids (e.g. pectin), phosphatides (e.g. lecithin), alginates (e.g. ammonium alginate, sodium, potassium or calcium alginate, propylene glycol alginate), gelatin, collagen, and cellulosics. Cellulosics are cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent.

In certain embodiments, the cellulosics for the erodible matrix comprises aqueous-soluble and aqueous-erodible cellulosics can include, for example, methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC). In certain embodiments, the cellulosics comprises various grades of low viscosity (MW less than or equal to 50,000 Daltons, for example, the Dow Methocel™ series E5, E15LV, E50LV and K100LY) and high viscosity (MW greater than 50,000 Daltons, for example, E4MCR, E10MCR, K4M, K15M and K100M and the Methocel™ K series) HPMC. Other commercially available types of HPMC include the Shin Etsu Metolose 90SH series.

Other materials useful as the erodible matrix material include, but are not limited to, pullulan, polyvinyl pyrrolidone (povidone), polyvinyl alcohol, polyvinyl acetate, glycerol fatty acid esters, polyacrylamide, polyacrylic acid, copolymers of ethacrylic acid or methacrylic acid (EUDRAGIT®, Evonik Health Care, Birmingham, Ala.) and other acrylic acid derivatives such as homopolymers and copolymers of butylmethacrylate, methylmethacrylate, ethylmethacrylate, ethylacrylate, (2-dimethylaminoethyl) methacrylate, and (trimethylaminoethyl) methacrylate chloride.

In some embodiments, the hydrophilic polymer is used as a binder in the unit dosage form and is selected from povidone, starch, hydroxypropylcellulose, and hydroxypropylmethylcellulose.

In some embodiments, the tablets used in the methods disclosed herein comprise a core and an enteric coating. The enteric coating surrounding the core may be applied using standard coating techniques. Materials used to form the enteric coating may be dissolved or dispersed in organic or aqueous solvents and may include one or more of the following: methacrylic acid copolymers; shellac; hydroxypropylmethylcellulose phthalate; polyvinyl acetate phthalate; hydroxypropylmethylcellulose trimellitate; carboxymethylcellulose; cellulose acetate phthalate; or other suitable enteric coating polymers. The pH at which the enteric coat will dissolve can be controlled by the polymer or combination of polymers selected and/or ratio of pendant groups. For example, dissolution characteristics of the coating can be altered by the ratio of free carboxyl groups to ester groups. Enteric coating layers may also contain pharmaceutical plasticizers such as: triethyl citrate; dibutyl phthalate; triacetin; polyethylene glycols; polysorbates; acetylated glycerides, etc. Additives such as dispersants, colorants, anti-adhering, taste-masking and anti-foaming agents may also be included. Any suitable enteric coating may be used. In certain embodiments, the enteric coating is a polyvinyl alcohol (PVA)-based coating composition such as Opadry® II 85 supplied by Colorcon. Opadry® Enteric is a platform of fully formulated delayed release coating systems from Colorcon.

The disclosed gastro-retentive dosage forms can be prepared by any suitable process, including by direct compression or by a dry granulation process. Methods of making the dosage forms and tablets used in the methods disclosed herein are known. See U.S. Pat. No. 9,205,094 and WO2014/113377.

In certain embodiments, prior to this treatment, the patient has not been completely responsive to other treatments, including individually optimized, standard-labeled dose daily PPI therapy for a minimum of 8 weeks prior to this treatment.

In certain embodiments, the patient has erosive esophagitis. In further embodiments, the patient has erosive esophagitis on esophagogastroduodenoscopy (EGD). In further embodiments, the patient has erosive esophagitis on EGD with approximately 48 to 96 hours of pH monitoring. In further embodiments, the pH monitoring is performed with a catheter-free, capsule-based pH monitoring device that is attached to the patient's esophagus (e.g., a Bravo® device). In other embodiments, the patient has evidence of pathological acid reflux on Esophagogastroduodenoscopy (EGD). In other embodiments, the patient has evidence of pathological acid reflux on EGD with approximately 48 to 96 hours of pH monitoring. In further embodiments, the pH monitoring is performed with a catheter-free, capsule-based pH monitoring device that is attached to the patient's esophagus (e.g., a Bravo® device). In further embodiments, the patient has pathological acid reflux with a pH of <4 for ≥4.2% during at least 1 of the 24-hour time intervals of pH testing with the catheter-free, capsule-based pH monitoring device that is attached to the patient's esophagus (e.g., a Bravo® device).

In certain embodiments, the tablets comprising the bile acid sequestrants are administered to the patient for eight weeks (eight treatment weeks), or more. The tablets may also be administered for less time, or until the patient's symptoms improve.

In certain embodiments, the patient experiences a clinically meaningful weekly heart burn severity score reduction compared to baseline. In certain embodiments, the patient experiences a clinically meaningful weekly heart burn severity score reduction compared to baseline of at least 30% for at least four of the eight treatment weeks, including at least one of the last two weeks. In certain embodiments, the patient experiences a clinically meaningful weekly heart burn severity score reduction compared to baseline of at least 45% for at least four of the eight treatment weeks, including at least one of the last two weeks. In certain embodiments, the patient experiences a clinically meaningful Weekly Regurgitation Frequency Score (WRFS) reduction compared to baseline. In certain embodiments, the patient experiences a clinically meaningful a clinically meaningful Weekly Regurgitation Frequency Score (WRFS) reduction compared to baseline of at least 30% for at least four of the eight treatment weeks, including one of the last two weeks. In certain embodiments, the patient experiences a clinically meaningful a clinically meaningful Weekly Regurgitation Frequency Score (WRFS) reduction compared to baseline of at least 45% for at least four of the eight treatment weeks, including one of the last two weeks.

In certain embodiments, the disclosed dosage form comprising the bile acid sequestrant is retained in the stomach until it is substantially or completely disintegrated.

In certain embodiments, the time for complete disintegration of the disclosed dosage form comprising the bile acid sequestrant is at least 1 hour, at least 2 hours, at least 3 hours, at least 4 hours, at least 4.7 hours, at least 5 hours, at least 5.5 hours, at least 5.55 hours, at least 6 hours, at least 6.4 hours, or at least 6.441 hours.

In certain embodiments, at least 50% of the dosage form comprising the bile acid sequestrant is retained in the stomach (not disintegrated) for at least 4.5 hours, at least 4,510 hours, at least 6.5 hours, at least 6.589 hours, at least 7 hours, or at least 7.069 hours.

In certain embodiments, at least 10% of the dosage form comprising the bile acid sequestrant is retained in the stomach (not disintegrated) for at least 6.4 hours, at least 6.432 hours, at least 8 hours, at least 8.394 hours, at least 9 hours, or at least 9.179 hours.

In certain embodiments, the patient takes PPI once daily.

The methods may be used to treat patients with a disease selected from heartburn, indigestion, dyspepsia, erosive esophagitis, peptic ulcer, gastric ulcer, esophageal ulcers, esophagitis, laryngitis, pharyngitis, coarse voice, gastroesophageal reflux disease (GERD), Barrett's esophagus, gastric cancer, esophageal cancer (e.g., adenocarcinoma), and gastritis and GERD-related pulmonary dysfunction, instead of, or in addition to, patients with symptomatic GERD not completely responsive to proton pump inhibitor.

Formulations and Compositions

This disclosure provides an enteric coated gastro-retentive, oral dosage form in the form of a tablet comprising: colesevelam or colesevelam hydrochloride dispersed in a polymeric matrix. In certain embodiments, the polymeric matrix comprising of or consists essentially of polyethylene oxide CAS Number 25322-68-3, approximate molecular weight 300,000 (PEG-7M). In some embodiments, the dosage form comprises one or more filler or compressing agent, which in certain embodiments, are selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate. The tablet is coated with an enteric coating, for prolonged retention of the bile acid sequestrant in the stomach of the patient. In certain embodiments, the disclosed oral dosage form comprises butylated hydroxytoluene (BHT). In certain embodiments, the disclosed oral dosage form comprises about 0.01 mg to about 1.5 mg of BHT. In certain embodiments, the disclosed oral dosage form comprises at least about 0.06% BHT by weight of the tablet core; the at least 0.065 BHT are added to formulation. These dosage forms may be used in the methods disclosed herein. In certain embodiments, the pharmaceutical composition further comprises an additional therapeutic agent. The enteric coated gastro-retentive, oral dosage forms in the form of a tablet are intended for oral delivery to a patient.

These dosage forms, formulations and pharmaceutical compositions are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dosage forms, formulations and pharmaceutical compositions disclosed herein are administered in a dose of 500 mg, 750 mg, 1,000 mg, 1,500 mg, 2100 mg or more (to the extent that it is safe). In certain embodiments, these dosage forms, formulations and pharmaceutical compositions are administered twice daily, each at a dose of 500 mg, 1,000 mg, or 1,500 mg, 700 mg, 1,400 mg, 2,100 mg, or more; in certain further embodiments, twice per day.

In certain embodiments, the dosage form may additionally contain suitable diluents, glidants, lubricants, acidulants, stabilizers, fillers, binders, plasticizers or release aids and other pharmaceutically acceptable excipients.

An ingredient of this polymeric matrix is at least one hydrophilic, water-swellable, erodible, or soluble polymer, which may generally be described as an “osmopolymer”, “hydrogel” or “water-swellable” polymer. More than one of such polymers may be combined in a dosage form of the invention to achieve gastric-retention as well as the desired erosion rate.

Polymers suitable for achieving the desired gastro-retentive and sustained-release profiles of the dosage forms of the invention have the property of swelling as a result of imbibing water from the gastric fluid, and gradually eroding over a time period of several hours. Since erosion of the polymer results from the interaction of the fluid with the surface of the dosage form, erosion initiates more or less simultaneously with the swelling process. While erosion and swelling may occur at the same time, the rate for achieving maximum swelling should be faster than the rate the dosage form fully erodes to achieve the desired release profile. Such polymers may be linear, branched, or cross linked. The polymers may be homopolymers or copolymers.

In some embodiments, the polymer is a polyalkylene oxide. In some embodiments, at least one of the one or more hydrophilic polymers is a polyethylene oxide (PEO). In still other embodiments, the at least one hydrophilic polymer is a polyethylene oxide having a molecular weight of about 300,000 Daltons.

In certain embodiments, the dosage form comprises one or more of microcrystalline cellulose (at between 1-10% w/w of the tablet core), butylated hydroxytoluene oxide (at between 0.01-0.10% w/w of the tablet core), colloidal silicon oxide (at between 1.0-2.5% w/w of the tablet core) and magnesium stearate (at between 0.1-1.0% w/w of the tablet core).

In certain embodiments, the enteric coating is a polyvinyl alcohol (PVA)-based coating composition such as Opadry® II 85 supplied by Colorcon. Opadry® Enteric is a platform of fully formulated delayed release coating systems from Colorcon. In certain embodiments, the tablets are coated with 1-4% Opadry® II 85F w/w of the coated tablet core.

Methods of making the dosage forms and tablets used in the methods disclosed herein are known. See U.S. Pat. No. 9,205,094 and WO2014/113377, the disclosures of both are incorporated by reference herein.

In certain embodiments, the oral dosage form is IW-3718. IW-3718 are tablets formulated as a gastric retentive, solid oral dosage form intended to extend the release of the drug substance, colesevelam, into the stomach. The released colesevelam binds bile acids that are refluxed into the stomach from the duodenum, rendering them functionally inert by forming a bile acid-colesevelam complex. This would prevent free bile acids from entering the esophagus and reduce residual GERD symptoms and mucosal damage resulting from bile reflux in GERD patients on PPI therapy. In certain embodiments, IW-3718 tablets contain colesevelam incorporated into a polymeric matrix. Such matrix may be an aqueous-erodible or water-swellable or aqueous-soluble in the sense of being either erodible or swellable or dissolvable (or combinations of these properties) in pure water or requiring the presence of an acid or base to ionize the polymeric matrix sufficiently to cause erosion or dissolution (e.g. gastric fluid). See U.S. Pat. No. 9,205,094 and WO2014/113377. In certain embodiments, IW-3718 tablets comprise polyethylene oxide (PEO). PEO is a linear polymer of unsubstituted ethylene oxide and has a wide range of viscosity-average molecular weights. An IW-3718 tablet used in Examples 1 and 2 is shown in Table 14.

In certain embodiments, the one or more filler or compressing agent of the oral dosage form comprising a bile acid sequestrant is microcrystalline cellulose at 1-10% w/w of the tablet core, butylated hydroxytoluene at 0.01 to 0.10% w/w of the tablet core, colloidal silicon dioxide at 1-5% w/w of the tablet core, and/or magnesium stearate at 0.1 to 1.0% w/w of the tablet core. In certain embodiments, the one or more filler or compressing agent is microcrystalline cellulose at 5.4% w/w of the tablet core, butylated hydroxytoluene at about 0.06 w/w of the tablet, colloidal silicon dioxide at 2.0% w/w of the tablet core, and/or magnesium stearate at 0.5% w/w of the tablet core.

In certain embodiments, the enteric coating of the oral dosage form comprising a bile acid sequestrant is a polyvinyl alcohol based enteric coating. In certain embodiments, the enteric coating of the oral dosage form comprising a bile acid sequestrant is a polyvinyl alcohol based enteric coating is Opadry® II 85F. In certain embodiments, the enteric coating of the oral dosage form comprising a bile acid sequestrant is a polyvinyl alcohol based enteric coating is Opadry® II 85F at 1-5% w/w of the tablet core. In further embodiments, the enteric coating is a polyvinyl alcohol based enteric coating is Opadry® II 85F at 3% w/w of the tablet core.

In certain embodiments, the PEG-7M (Polyox™ WSR N-750) is at 30 to 60% w/w of the tablet core. In further embodiments, the PEG-7M (Polyox™ WSR N-750) is at 46% w/w of the tablet core.

Combination Therapies

The methods disclosed herein may be used to treat patients using combination therapy, comprising administering a gastric-retentive oral dosage forms comprising at least one bile acid sequestrant in combination with one or more additional therapeutic agents. For combination treatment with more than one active agent, where the active agents may be in separate dosage forms, the active agents may be administered separately or in conjunction. In addition, the administration of one agent may be prior to, concurrent to, or subsequent to the administration of the other agent.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

In some embodiments, the methods further comprise administering to the patient simultaneously, separately, or sequentially, one or more proton pump inhibitors (PPI). In certain embodiments, the PPI is administered QD (once-per-day). In other embodiments, the methods further comprise administering simultaneously, separately or sequentially, one or more acid pump antagonists. In other embodiments, the methods further comprise administering simultaneously, separately, or sequentially one or more agents chosen from an antacid, a histamine H2-receptor antagonist, a γ-aminobutyric acid-β (GABA-B) agonist, a prodrug of a GABA-B agonist, and a protease inhibitor.

While the two or more agents in the combination therapy can be administered simultaneously, they need not be. For example, administration of a first agent (or combination of agents) can precede administration of a second agent (or combination of agents) by minutes, hours, days, or weeks. Combination therapy can also include two or more administrations of one or more of the agents used in the combination.

PPI drugs are substituted benzimidazole compounds that specifically inhibit gastric acid secretion by affecting the H⁺/K⁺ ATPase enzyme system (the proton pump). These drugs, for example esomeprazole, are rapidly absorbed and have very short half-lives. However, they exhibit prolonged binding to the H⁺/K⁺ ATPase enzyme. The anti-secretory effect reaches a maximum in about 4 days with once-daily dosing. Because of these characteristics, patients beginning PPI therapy do not receive maximum benefit of the drug and healing may not begin for up to 5 days after therapy begins when PPIs are used alone for initial therapy of upper GI tract disorders.

Proton pump inhibitors (PPIs) are potent inhibitors of gastric acid secretion, inhibiting H⁺/K⁺ ATPase, the enzyme involved in the final step of hydrogen ion production in the parietal cells. The term proton pump inhibitor includes, but is not limited to, omeprazole (as sold under the brand-names PRILOSEC®, LOSEC, or ZEGERID®), lansoprazole (as sold under the brand-name PREVACID®, ZOTON®, or INHIBITOL®), rabeprazole (as sold under the brand-name RABECID®, ACIPHEX®, or PARIET®), pantoprazole (as sold under the brand-name PROTONIX®, PROTIUM®, SOMAC®, or PANTOLOC®), tenatoprazole (also referred to as benatoprazole), and leminoprazole, including isomers, enantiomers and tautomers thereof (e.g., esomeprazole (as sold under the brand-name NEXIUM®)), Dexlansoprazole, Dexrabeprazole, (S)-Pantoprazole, Ilaprazole and alkaline salts thereof; The following patents describe various benzimidazole compounds suitable for use in the disclosure described herein: U.S. Pat. Nos. 4,045,563, 4,255,431, 4,359,465, 4,472,409, 4,508,905, JP-A-59181277, U.S. Pat. Nos. 4,628,098, 4,738,975, 5,045,321, 4,786,505, 4,853,230, 5,045,552, EP-A-295603, U.S. Pat. No. 5,312,824, EP-A-166287, U.S. Pat. No. 5,877,192, EP-A-519365, EP5129, EP 174,726, EP 166,287 and GB 2,163,747. Thus, proton pump inhibitors and their pharmaceutically acceptable salts, which are used in accordance with the present disclosure, are known compounds and can be produced by known processes. In certain embodiments, the proton pump inhibitor is omeprazole, either in racemic mixture or only the (−) enantiomer of omeprazole (i.e. esomeprazole), as set forth in U.S. Pat. No. 5,877,192, hereby incorporated by reference.

Omeprazole is typically administered in a 20 mg dose/day for active duodenal ulcer for 4-8 weeks; in a 20 mg dose/day for gastro-esophageal reflux disease (GERD) or severe erosive esophagitis for 4-8 weeks; in a 20 mg dose/twice a day for treatment of Helicobacter pylori (in combination with other agents); in a 60 mg dose/day for active duodenal ulcer for 4-8 weeks and up to 120 mg three times/day, and in a 40 mg dose/day for gastric ulcer for 4-8 weeks. In other embodiments of the present disclosure, the dose of proton pump inhibitor is sub-therapeutic.

Lansoprazole is typically administered about 15-30 mg/day; rabeprazole is typically administered 20 mg/day and pantoprazole is typically administered 40 mg/day. However, any therapeutic or sub-therapeutic dose of these agents is considered within the scope of the present disclosure.

Acid pump antagonists (APAs) acting by K(+)-competitive and reversible (as opposed to irreversible PPIs) binding to the gastric proton pump, which is the final step for activation of acid secretion in the parietal cell. One class of APAs are imidazopyridines. Examples of some APAs include, but are not limited to: BY-841 (Prumaprazole), Sch-28080, YJA-20379-8, YJA-20379-1, SPI-447, SK&F-97574, AU-2064, SK&F-96356, T-330, SK&F-96067, SB-641257A (YH-1885, Revaprazan hydrochloride, RevanexR), CS-526, R-105266, Linaprazan, Sorapraza, DBM-819, KR-60436, RQ-00000004 (RQ-4) and YH-4808.

Other agents include: histamine H2 receptor blockers, motility agents (gastroprokinetics), antacids, antiulcerative agents, γ-aminobutyric acid-0 (GABA-B) agonists, prodrugs of GABA-B agonists, GCC agonists and/or protease inhibitors. Non-limiting examples of these additional agents include: cinitapride, cisapride, fedotozine, loxiglumide, alexitol sodium, almagate, aluminum hydroxide, aluminum magnesium silicate, aluminum phosphate, azulene, basic aluminum carbonate gel, bismuth aluminate, bismuth phosphate, bismuth subgallate, bismuth subnitrate, calcium carbonate, dihydroxyaluminum aminoacetate, dihydroxyaluminum sodium carbonate, ebimar, magaldrate, magnesium carbonate hydroxide, magnesium hydroxide, magnesium oxide, magnesium peroxide, magnesium phosphate (tribasic), magnesium silicates, potassium citrate, sodium bicarbonate, aceglutamide aluminum complex, acetoxolone, aldioxa, arbaprostil, benexate hydrochloride, carbenoxolone, cetraxate, cimetidine, colloidal bismuth subcitrate, ebrotidine, ecabet, enprostil, esaprazole, famotidine, gefamate, guaiazulene, irsogladine, misoprostol, nizatidine, omoprostil, -Oryzanol, pifamine, pirenzepine, plaunotol, polaprezinc, ranitidine, rebamipide, rioprostil, rosaprostol, rotraxate, roxatidine acetate, sofalcone, spizofarone, sucralfate, telenzepine, teprenone, trimoprostil, trithiozine, troxipide, zolimidine, baclofen, R-baclofen, XP19986 (CAS Registry No. 847353-30-4), pepstatin and other pepsin inhibitors (e.g., sodium benzoate); and chymotrypsin and trypsin inhibitors. A wide variety of trypsin and chymotrypsin inhibitors are known to those skilled in the art and can be used in the methods described herein. Such trypsin and chymotrypsin inhibitors can include tissue-factor-pathway inhibitor; α-2 antiplasmin; serpin α-1 antichymotrypsin family members; gelin; hirustasin; eglins including eglin C; inhibitors from Bombyx mori (see; e.g.; JP 4013698 A2 and JP 04013697 A2; CA registry No. 142628-93-1); hirudin and variants thereof; secretory leukocyte protease inhibitor (SLPI); α-1 anti-trypsin; Bowman-Birk protease inhibitors (BBIs); chymotrypsin inhibitors represented by CAS registry Nos. 306762-66-3, 306762-67-4, 306762-68-5, 306762-69-6, 306762-70-9, 306762-71-0, 306762-72-1, 306762-73-2, 306762-74-3, 306762-75-4, 178330-92-2, 178330-93-3, 178330-94-4, 81459-62-3, 81459-79-2, 81460-01-7, 85476-59-1, 85476-62-6, 85476-63-7, 85476-67-1, 85476-70-6, 8585866-8, 85858-68-0, 85858-69-1, 85858-70-4, 85858-71-5, 85858-72-6, 85858-73-7, 85858-75-9, 85858-77-1, 85858-79-3, 85858-81-7, 85858-83-9, 85858-84-0, 85858-85-1, 85858-87-3, 85858-89-5, 85858-90-8, 85858-92-0, 85879-03-4, 85879-05-6, 85879-06-7, 85879-08-9, 85858-74-8, 90186-24-6, 90185-93-6, 89703-10-6, 138320-33-9 (YS3025), 94149-41-4 (MR889), 85858-76-0, 89703-10-6, 90185-92-5, 90185-96-9, 90185-98-1, 90186-00-8, 90186-01-9, 90186-05-3, 90186-06-4, 90186-07-5, 90186-08-6, 90186-09-7, 90186-10-0, 90186-11-1, 90186-12-2, 90186-13-3, 90186-14-4, 90186-22-4, 90186-23-5, 90186-24-6, 90186-25-7, 90186-27-9, 90186-28-0, 90186-29-1, 90186-31-5, 90186-35-9, 90186-43-9, 90209-88-4, 90209-89-5, 90209-92-0, 90209-94-2, 90209-96-4, 90209-97-5, 90210-01-8, 90210-03-0, 90210-04-1, 90210-25-6, 90210-26-7, 90210-28-9, 90230-84-5, 90409-84-0, 95460-86-9, 95460-87-0, 95460-88-1, 95460-89-2, 95460-91-6, 114949-00-7, 114949-01-8, 114949-02-9, 114949-03-0, 114949-04-1, 114949-05-2, 114949-06-3, 114949-18-7, 114949-19-8, 114964-69-1, 114964-70-4, 9076-44-2 (chymostatin), 30827-99-7 (Pefabloc), 618-39-3 (benzamidine), 80449-31-6 (urinistatin), 130982-43-3, 197913-52-3, 179324-22-2, 274901-16-5, 792163-40-7, 339169-59-4, 243462-36-4, 654671-78-0, 55123-66-5 (leupeptin), 901-47-3, 4272-74-6, 51050-59-0, 221051-66-7, 80449-31-6, 55-91-4, 60-32-2, 88070-98-8, 87928-05-0, 402-71-1 (benzenesulfonamide), 139466-47-0, CI-2A (see U.S. Pat. No. 5,167,483), CI-2A (see bWO9205239), WCI-3 (see Shibata et al. 1988 J Biochem (Tokyo) 104:537-43), WCI-2 (see Habu et al. 1992 J Biochem (Tokyo) 111:249-58), and WCI-x (Habu et al., supra) and 178330-95-5; and compounds with chymotrypsin inhibition activity described in patent publications JP 56092217 A2, U.S. Pat. Nos. 4,755,383, 4,755,383, 4,639,435, 4,620,005, 4,898,876, and EP0128007.

Examples of other therapeutic agents that may be combined with a compound of this disclosure, either administered separately or in the same pharmaceutical composition, include, but are not limited to linaclotide, IW-9179, plecanatide and SP-333.

Any additional suitable agents may be administered to the patient.

### EXAMPLES

For this invention to be better understood, the following examples are set forth. These examples are for purposes of illustration only and are not be construed as limiting the scope of the invention in any manner.

### Example 1: A Randomized, Double-Blind, Placebo-Controlled, Parallel-Group, Dose-Range-Finding Trial to Determine Safety and Efficacy of a Bile Acid Sequestrant Dosage Form Administered Orally for 8 Weeks to Patients with Symptomatic Gastroesophageal Reflux Disease not Completely Responsive to Proton Pump Inhibitors

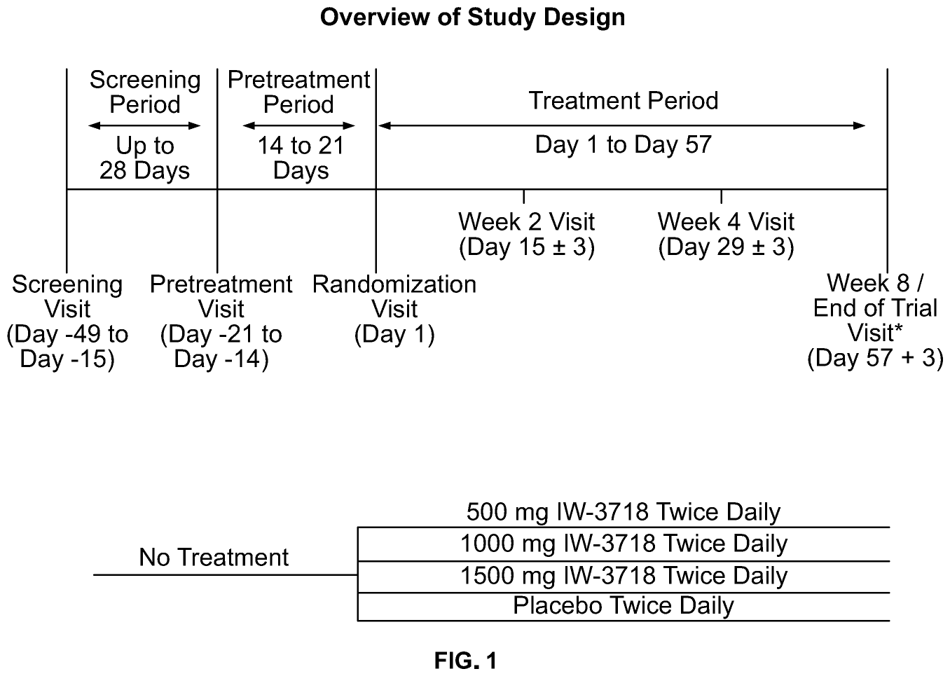

This Example presents protocol and results of a multicenter (approximately 60-80 centers in the United States), randomized, double-blind, placebo-controlled, parallel-group, 8-week study, consisting of 3 distinct periods (a screening period of up to 28 days; a pretreatment period of 14-21 days; and a treatment period of 57 days). FIG. 1. The study enrolls patients who have GERD and continue to experience GERD symptoms while receiving QD, standard-dose PPI therapy that in the investigator's opinion has been optimized. Eligible patients continue to take their PPI and are to be randomized to placebo or to 500 mg IW-3718 twice daily (BID), 1000 mg IW-3718 BID, or 1500 mg IW-3718 BID.

IW-3718 tablets are formulated as a solid oral dosage form intended to extend the release of the drug substance, colesevelam, into the stomach. The released colesevelam binds bile acids that are refluxed into the stomach and upper duodenum, forming a bile acid-colesevelam complex and preventing the free bile acids from entering the esophagus. The bile acid-colesevelam complex travels down the GI tract and is excreted without being absorbed. The controlled-release formulation in IW-3718 tablets is based on Depomed's Acuform® technology which utilizes swelling polymers to allow the tablet to be retained in the stomach for approximately 9 hours when dosed in the fed state, during which time the tablet slowly releases the active ingredient in the stomach and upper GI tract. The tablet's active ingredient is steadily delivered to the stomach and upper GI tract in a near zero-order manner. The technology is used in the formulation of 5 Food and Drug Administration (FDA)-approved drugs: Glumetza® (metformin HCl, extended release), Janumet® XR (sitagliptin and metformin HCl extended-release), Proquin® XR (ciprofloxacin HCl monohydrate, extended release), NUCYNTA® ER (tapentadol extended-release tablets), and once-daily Gralise™ (gabapentin) tablets. Colesevelam is not systemically absorbed and does not interfere with systemic drug metabolizing enzymes. Distribution of colesevelam is limited to the GI tract and elimination occurs through fecal excretion.

The objectives are to evaluate the safety, efficacy, and dose-response relationship of IW-3718 administered orally to patients who have GERD and continue to experience GERD symptoms while receiving once-daily (QD), standard-dose PPIs.

Eligible patients continue to take their PPI and are randomized to placebo or to 500 mg IW-3718 twice daily (BID), 1000 mg IW-3718 BID, or 1500 mg IW-3718 BID. Randomization is stratified by whether they have, or do not have, erosive esophagitis on the screening esophagogastroduodenoscopy (EGD).

All patients are to continue to take their current PPI approximately 30-60 minutes before breakfast each day during the Screening, Pretreatment, and Treatment Periods. During the Treatment Period, all patients take IW-3718 or matching placebo (PBO) immediately upon completion of the morning and evening meal each day.

Screening Period: The Screening Period starts with the signature of the informed consent form (ICF) and is expected to last for up to 28 days. During this period, patient eligibility for entry into the Pretreatment Period is to be determined. Two procedures are required during the Screening Period in all patients; a third procedure Bilitec® testing is optional for all patients at selected sites (all is to be done while patients continue to take their PPI):

EGD

Approximately 48 to 96 hours of pH testing with the Bravo® device. If for some reason 96 hours of testing is not possible, then approximately 48 hours of testing is acceptable

Approximately 24 hours of bilirubin detection using the Bilitec monitoring system (optional at selected sites)

Histamine-2 receptor antagonists (H2RAs) should be stopped 5 calendar days prior to the EGD and Bravo® pH monitoring and antacids should be stopped 1 calendar day prior to the EGD and Bravo® pH monitoring. The EGD must be performed during the Screening Period and at least 7 days before the start of the Pretreatment Period to allow time for pH collection and allow the patient to stabilize following these procedures. Upon completion of the Bravo testing, patients are to refrain from using H2RAs, but may continue to use antacids if needed. Patients are to continue to use their current PPI throughout the Screening Period. The end of the Screening Period coincides with the start of the Pretreatment Period.

At selected sites, patients who are screened for the study with EGD and Bravo testing are also given the option of having a Bilitec monitor inserted at that time. The Bilitec procedure is being completed in a subset of patients to collect information about whether bilirubin is present in the duodenogastric refluxate. Previous Bilitec 2000 studies have shown associations between total bile acid concentrations and Bilitec 2000 absorbance. Patients participating in the Bilitec procedure agree to comply with a “white diet,” defined below, and return approximately 24 hours later for removal of the probe. The results of the Bilitec monitoring do not affect qualification for enrollment and during participation in the study, patients are to be blinded of their Bilitec testing results.

A one-week extension of the screening period window is granted when needed for logistical delays.

Pretreatment Period: The Pretreatment Period is defined as the 14 to 21 calendar days immediately before the Randomization Visit. During this period, patients continue to use their PPI and refrain from using other anti-reflux medications, including antacids and H2RAs, except for the antacid that is dispensed as rescue medicine. They provide the following information in a handheld eDiary; perform at least 2 weeks of symptom assessments during which they are required to complete daily assessments for at least 5 days each week during the 14 calendar days before the start of the Treatment Period and weekly assessments at least once during the 7 calendar days before the start of the Treatment Period to be eligible for randomization:

GERD symptoms assessed once daily in the evening in the modified Reflux Symptom Questionnaire eDiary (mRESQ-eD—Modified Reflux Symptom Questionnaire, from Endpoint Outcomes)

Dyspepsia symptoms assessed once daily in the evening using Daily Dyspepsia Symptoms

Assessment of sleep assessed once daily in the morning using Daily Assessment of Sleep

Use of per-protocol rescue medicine assessed twice daily

Symptom relief and “bothersomeness” assessed once a week in the evening

Patients are instructed to take their PPI at least approximately 30-60 minutes before breakfast each day, even on study visit days. Patients who satisfy all the entry criteria enter the Treatment Period.

Treatment Period: The Treatment Period begins with treatment assignment and lasts for 8 weeks. Patients are stratified by whether they have, or do not have, erosive esophagitis on the screening EGD and randomly assigned to 1 of 4 treatments (1:1:1:1) within each stratum: placebo or 500 mg IW-3718 BID, 1000 mg IW-3718 BID, or 1500 mg IW-3718 BID. Enrollment is monitored to ensure that no single center contributes >15% of the targeted study enrollment, unless otherwise approved by the Medical Monitor. Study drug is taken immediately after the morning and evening meals. Patients are to continue to take their PPI approximately 30-60 minutes before breakfast each day and to use the eDiary to provide their daily assessments (GERD symptoms, dyspepsia symptoms, assessment of sleep), weekly assessments (weekly symptom bothersomeness and degree of relief questions), PPI compliance, and use of per-protocol rescue medicine.

A double-blind, placebo-controlled, parallel-group study design was chosen in accordance with the concepts in International Conference on Harmonization (ICH) E10, Choice of Control Groups and Related Issues in Clinical Trials (International Conference on Harmonization of Technical Requirements for Registration of Pharmaceuticals for Human Use, 2001), to provide comparable treatment groups and minimal chance of selection or Investigator bias.

This study has a 14- to 21-day Pretreatment Period to establish a baseline without therapy and to familiarize patients with data collection methodology (i.e., personal digital assistants [PDAs]), and an 8-week Treatment Period to compare the test treatment with a placebo control.

Selection of Study Population

This study is conducted in patients with GERD not completely responsive to QD PPI therapy. Approximately 260 patients are randomized (approximately 65 patients per treatment group). Study medication is administered orally BID for 8 weeks; total patient participation is expected to last up to 109 days, including the Screening Period.

Eligibility Criteria: Inclusion Criteria

Each patient must meet all the following criteria to be eligible for enrollment in this study:

1. Patient has signed an informed consent form (ICF) before any study-specific procedures are performed.

2. Patient is an ambulatory, community-dwelling male or nonpregnant female and is at least 18 years old at the Screening Visit. Lactating females must agree not to breastfeed.

3. Patient has a diagnosis of GERD and reports experiencing GERD symptoms (heartburn or regurgitation) on ≥4 days per week during the 8 weeks before the Screening Visit while taking standard QD PPI therapy.

4. Patient has been receiving individually optimized, standard-labeled dose, QD, PPI therapy (treatment that, according to the Investigator's judgment, could not be further improved by changing the brand or timing of PPI administration) for a minimum of 8 weeks before the Screening Visit. Patients should be on a PPI dose and schedule that is consistent with the approved labeling. Patients who have their PPI modified during the Screening Period may be re-screened after 8 weeks of optimized, standard-labeled dose, QD (one-a-day), PPI therapy provided they have not previously entered the Pretreatment Period.

5. An EGD with approximately 48 to 96 hours of pH monitoring (with a Bravo® device) during the Screening Period (while the patient continues taking their PPIs) demonstrates one or more of the following:

a. Erosive esophagitis (Grade A or greater based on the Los Angeles classification of esophagitis) on EGD

b. Evidence of pathological acid reflux (pH is <4 for ≥4.2% of the recording time) during at least 1 of the 24-hour time intervals of pH testing with the Bravo® device

6. Patient reports heartburn severity (HS, mRESQ-eD)≥3 (moderate) on at least 2 days and has an average HS of ≥2 (mild) during the last 7 days before randomization.

7. Sexually active female patients of childbearing potential (i.e., women who are not postmenopausal or who have not had a bilateral oophorectomy, hysterectomy, or tubal ligation) must agree to use 1 of the following methods of birth control from the date they sign the ICF until 24 hours after their final dose of study drug:
a. Hormonal contraception (i.e., contraceptive implant or injectable hormonal contraceptive)
b. Double-barrier birth control (e.g., condom plus intrauterine device, diaphragm plus spermicide, etc.) Patients may continue taking oral contraceptives while using one or more barrier methods.
c. Maintenance of a monogamous relationship with a male partner who has been surgically sterilized by vasectomy

8. Females of childbearing potential must have a negative serum pregnancy test at the Screening Visit and a negative urine pregnancy test at the Randomization Visit prior to dosing.

9. Patient agrees not to make any changes to their usual diet during the study, except those patients participating in Bilitec testing, who must agree to a “white diet” during Bilitec testing only.

10. Patient is compliant with eDiary completion; that is, they have adequately completed the eDiary questions on at least 5 days each week during the 14 calendar days before the start of the Treatment Period.

11. Patient is compliant with QD PPI dosing during the 14 calendar days before the start of the Treatment Period. Patients are considered compliant if, as reported in the eDiary, they take their PPI on at least 5 days each week.

12. Patient is fluent and literate in English or Spanish.

13. Patient is able to operate the eDiary adequately and agrees to adhere to the study requirements.

14. For patients who are receiving supplementation of a fat-soluble vitamin in order to correct or avoid a fat-soluble vitamin deficiency, the patient is willing to take the vitamin supplement at least 4 hours before taking study medication.

Eligibility Criteria: Exclusion Criteria

1. Patient has a history of complete lack of GERD symptom response to PPIs in the past.

2. Patient reports epigastric pain or burning as his or her predominant symptom at the Screening Visit.

3. Patient has a history of gastroparesis, bowel obstruction, or is at risk for a bowel obstruction (e.g., patient has an organic GI motility disorder or a history of major GI surgery).

4. Patient has a history of serum triglycerides concentrations>500 mg/dL on a fasting specimen, or has serum triglycerides concentrations>500 mg/dL on a fasting specimen at Screening or any time during the Pretreatment Period.

5. Patient has a history of hypertriglyceridemia-induced pancreatitis.

6. In the Investigator's opinion, patient is susceptible to a deficiency of fat-soluble vitamins (especially vitamin D deficiency; e.g., the patient is African American or Hispanic or has osteoporosis, osteomalacia, etc.) and is put at risk by receiving colesevelam for 8 weeks.

7. Patient has an active swallowing disorder that would prevent her from being able to swallow the study medication.

8. Patient has any alarm symptoms including but not limited to GI bleeding, anemia, vomiting, or unexpected weight loss any time during the Screening or Pretreatment Periods.

9. Patient has undergone surgery that meets any of the following criteria:

a. Surgery of the GI tract (including gastric banding) other than an appendectomy, holecystectomy, minor oral or rectal surgery (e.g., tonsillectomy, hemorrhoidectomy, rectocele repair) at any time before the Screening Visit
b. An appendectomy during the 3 months before the Screening Visit or a cholecystectomy during the 6 months before the Screening Visit or minor oral or rectal surgery during the 30 days before the Screening Visit
c. Non-GI surgery of the abdomen, pelvis, or retroperitoneal structures during the 6 months before the Screening Visit
d. Thoracic surgery during the 6 months before the Screening Visit
e. Other major non-GI surgery during the 30 days before the Screening Visit

10. Patient has previously undergone thoracic or abdominal radiotherapy.

11. EGD, conducted during the Screening Period, reveals that the patient has long-segment Barrett's esophagus (greater than 3 centimeters) or definite dysplastic changes in the esophagus, peptic ulcer disease, active GI bleeding, presence of symptomatic esophageal strictures, presence of esophageal or fundic varices, erosive gastritis, or eosinophilic, herpetic or candida esophagitis.

12. Patient has Gilbert's disease, Crohn's disease, diabetes mellitus, Zollinger-Ellison syndrome, pancreatitis, cholecystitis, or systemic sclerosis.

13. Patient has elevated (defined as >1.5 times the upper limit of normal by the laboratory) levels of serum bilirubin at Screening or anytime during the Pretreatment Period.

14. Patient has a history of clinically significant hypersensitivity or allergies to any of the excipients contained in the study medication (active or placebo).

15. Patient has a history of cancer (resected basal cell or squamous cell carcinoma is acceptable). Note: patients with a history of cancer are allowed provided that the malignancy has been in complete remission for at least 5 years before the Screening Visit. A complete remission is defined as the disappearance of all signs of cancer in response to treatment.

16. Patient has history of active alcoholism, drug addiction, or illicit drug use (including marijuana) during the 12 months before the Screening Visit.

17. Patient has any clinically significant finding on a physical exam, 12-lead electrocardiogram (ECG), or clinical laboratory test after signing the ICF but before receiving the first dose of study medication. (Note: The Investigator determines if a particular finding is clinically significant.) In making this determination, the Investigator considers whether the particular finding could prevent the patient from performing any of the protocol-specified assessments, could represent a condition that would exclude the patient from the study, could represent a safety concern if the patient participates in the study, or could confound the study-specified assessments of safety or efficacy.)

18. Patient reports using a prohibited medication during the Screening or Pretreatment Periods, or is not willing or able to abide by the restrictions regarding use of prohibited medications.

19. Patient has received an investigational drug during the 30 days before the Screening Visit, or is planning to receive another investigational drug or use an investigational device at any time during the study.

20. Patient has an acute or chronic condition that, in the Investigator's opinion, would limit the patient's ability to complete or participate in this clinical study.

21. Patient has previously entered the Treatment Period of study in which IW-3718 is a treatment.

22. Patient has previously entered the Pretreatment Period of this study.

23. Patient is enrolled in this study at another clinical study site; is an employee of the Institution or Ironwood Pharmaceuticals; or is a first-degree family member, significant other, or relative residing with an employee of the Institution or Ironwood Pharmaceuticals.

Removal of Patients from Therapy or Assessment

A premature discontinuation occurs when a patient who has signed the ICF ceases participation in the study, regardless of circumstances, before completion of the Treatment Period.

A patient is considered to have completed the study after receiving 8 weeks of treatment and completing the End-of-treatment (EOT) Visit at Day 57. A window of +3 days is allowed for the EOT visit; if a patient completes the EOT Visit prior to Day 57, it is considered a protocol deviation.

Patients is informed that they are free to withdraw from the study at any time and for any reason. The Investigator may remove a patient from the study if, in the Investigator's opinion, it is not in the best interest of the patient to continue the study. Patients may also be discontinued from the study by the Investigator or the Sponsor at any time for any reason, including the following:
Failure to meet an inclusion/exclusion criterion that is clinically relevant and affects patient safe
Adverse event(s)
Protocol violation, including lack of compliance
Insufficient therapeutic response (lack of efficacy)
Lost to follow-up (every effort must be made to contact the patient; a certified letter must be sent)
Withdrawal of consent (attempts should be made to determine the reason for the patient withdrawing consent if possible)
Study termination by the Sponsor
Other reasons (e.g., administrative reasons or pregnancy)

Patients who discontinue from the study for any reason should complete the assessments required at the EOT Visit at the time of their discontinuation. Patients who discontinue from the study are followed until resolution of all their AEs or until the unresolved AEs are judged by the Investigator to have stabilized. Study centers should make a reasonable effort to follow any pregnant patients until delivery or end of the pregnancy.

If a patient does not return for a scheduled visit, the study center should contact the patient. An effort must be made to contact the patient, including sending a certified letter. In every case, the patient outcome, including lost to follow-up information, is documented.

Patients in this study who prematurely discontinue treatment are not be replaced.

Study Treatments

Study medication is provided as 500 mg IW-3718 tablets, which are white to off-white, oval shaped, film-coated tablets intended for oral administration. In addition to the active drug substance, colesevelam, 500 mg IW-3718 tablets contain the following inactive ingredients: microcrystalline cellulose, polyethylene oxide, colloidal silicon oxide, butylated hydroxyltoluene, magnesium stearate, polyvinyl alcohol, polyethylene glycol, titanium dioxide, and talc. Tablets should be taken whole and never broken, crushed, or chewed. See also Table 14.

Patients randomized to receive IW-3718 are administered the test product as follows:
500 mg IW-3718 BID: One 500 mg IW-3718 oral tablet plus 2 placebo oral tablets (3 tablets total) administered BID, immediately after the morning and evening meals
1000 mg IW-3718 BID: Two 500 mg IW-3718 oral tablets plus 1 placebo oral tablet (3 tablets total) administered BID, immediately after the morning and evening meals
1500 mg IW-3718 BID: Three 500 mg IW-3718 oral tablets administered BID, immediately after the morning and evening meals

Placebo to match 500 mg IW-3718 tablets are provided as white to off-white, oval shaped, film-coated, oral tablets containing microcrystalline cellulose, polyethylene oxide, colloidal silicon oxide, butylated hydroxyl toluene, magnesium stearate, polyvinyl alcohol, polyethylene glycol, titanium dioxide, and talc. Tablets should be taken whole and never broken, crushed, or chewed.

Patients randomized to receive placebo are administered three oral placebo tablets BID immediately after the morning and evening meals.

Antacid rescue medicine is provided to the clinical site as a bottled liquid (magnesium hydroxide 200 mg/aluminum hydroxide 200 mg per 5 mL).

During completion of the PDA daytime and nighttime diary questions, patients are asked about their rescue medicine use by responding to the following questions:
“How many times did you use your rescue medicine (liquid antacid) from the time you got out of bed this morning until now?” (Daytime assessment, completed at night before going to bed)
“How many times did you use your rescue medicine (liquid antacid) last night?” (Nighttime assessment, completed in the morning)

Treatment Compliance

Patients are dispensed the appropriate number of Sponsor-packaged, labeled blister wallets needed until the next study visit. Patients are asked to return all blister wallets (including unused tablets) at each study visit for assessment of compliance with the dosing regimen. Patients need to record their PPI administration each day (once daily) in their eDiary.

Packaging and Labeling

Study medication (IW-3718 and placebo tablets) is provided by Ironwood Pharmaceuticals as 60 count blisters in wallets, indicating morning and evening doses. Study medication is uniquely numbered and labeled in a double-blind fashion that conforms to regulatory requirements.

Study medication (IW-3718 and placebo tablets) is shipped at refrigerated temperatures between 2° C. and 8° C. (36° F. and 46° F.) and must be stored at refrigerated temperatures between 2° C. and 8° C. (36° F. and 46° F.). Antacid rescue medicine is shipped at room temperature between 20° C. and 25° C. (68° F. and 77° F.) and must be stored at room temperature between 20° C. and 25° C. (68° F. and 77° F.). Any deviation from these storage conditions must be reported and use of the study medication suspended until authorization for its continued use has been provided.

The Investigator must ensure that the receipt and use of all study medication supplied is recorded and must supervise the storage and allocation of these supplies. All study medication supplies must be retained in a locked room that may only be accessed by the Investigator, or other duly designated persons. Study medication must not be used outside the context of this protocol, and under no circumstances should the Investigator or study center personnel allow the supplies to be used other than as directed by this protocol.

Method of Assigning Patients to Treatment Groups

Patients who meet all the inclusion criteria and none of the exclusion criteria are randomized into the study on Day 1. Patients are stratified by whether they have or do not have erosive esophagitis on the screening EGD then randomized through central randomization in a 1:1:1:1 ratio to receive either 500 mg IW-3718 BID, 1000 mg IW-3718 BID, 1500 mg IW-3718 BID, or placebo BID.

The computer-generated randomization schedule is prepared by an independent statistician not otherwise associated with the study.

During this study, oral doses of IW-3718 at 500 mg BID (total daily dose of 1000 mg/day), 1000 mg BID (total daily dose of 2000 mg/day), and 1500 mg BID (total daily dose of 3000 mg/day) given as an adjunct to QD PPI for 56 days are studied to evaluate the dose-response relationship for IW-3718 in a placebo-controlled study. The 1000 mg BID dose was used in a previous Phase 2a study (Study ICP-3718-201) and provided a reasonable level of efficacy with an acceptable safety and tolerability profile. The highest dose level (1500 mg BID) was chosen to determine whether it can provide a higher level of efficacy with an acceptable safety profile. The lowest dose level (500 mg BID) is included since it may demonstrate efficacy, and allows for a more accurate assessment of the dose-response relationship for IW-3718. The safety profile at all 3 doses is also evaluated.

Patients are randomized to receive 500 mg IW-3718 BID, 1000 mg IW-3718 BID, 1500 mg IW-3718 BID, or placebo BID. The first dose of study medication is taken with liquid and a snack in clinic at the Randomization Visit (on Day 1). Patients are to take their second dose that evening immediately upon completion of dinner, ensuring that at least 8 hours have elapsed since the first dose in clinic.

During the Treatment Period, patients are to take study medication BID at home, in the morning (immediately upon completion of breakfast) and in the evening (immediately upon completion of dinner), even on study visit days. The last dose of study medication is taken the morning of the EOT Visit.

Additionally, all patients are to take their current PPI during the Pretreatment, Randomization, and Treatment Periods (i.e., Day −21 through Day 57 [+3 days]). The PPI is to be taken each day, approximately 30-60 minutes before breakfast.

During completion of the PDA daily diary questions, patients are asked to confirm daily dosing of their PPI.

Blinding

This study is double-blind and placebo-controlled.

Unblinding of a patient's treatment assignment is restricted to emergency situations and should only be used under circumstances where knowledge of the treatment is necessary for the proper handling of the patient. Except in a medical emergency, the Investigator and blinded study center staff remain blinded during the conduct of the study and until such time that all discrepancies in the clinical database are resolved (i.e., at the time of the database lock).

Individual patient treatment assignment unblinding is available to the Investigator through the interactive web response system (IWRS) in the event of an emergency. The Investigator should make all reasonable efforts to notify and discuss the circumstances requiring unblinding with the Medical Monitor or designee in advance of breaking the blind. If the treatment blind is broken, the reason and the date should be recorded and signed by the Investigator and information regarding the unblinding should be submitted as soon as possible to the Sponsor. The patient is immediately withdrawn from the study if the code is broken. The Sponsor may also break the blind in circumstances where unblinding is necessary for the safety of the patients.

Prior and Concomitant Medications

At the Screening Visit, the following information are recorded for each patient:
All medications the patient is taking (ongoing)
All prior medications taken during the 30 days before the Screening Visit
Most recent use of an H2RA
Most recent use of an antacid

Any medication taken by a patient during the course of the study (beginning at the Screening Visit), including any new medications added or changes in medications previously reported, and the reason for its use.

Rescue Medication

During the Pretreatment and Treatment Periods, patients may use dispensed, protocol-permitted antacid (magnesium hydroxide 200 mg/aluminum hydroxide 200 mg per 5 mL; 15 mL up to 4 times/day) as rescue medicine when their heartburn becomes intolerable. Each day, patients record in their eDiary the number of times that rescue medication was used.

Prohibited Medicines

Medicines that are not permitted during the Pretreatment and Treatment Periods are provided below.

Dietary Requirements

Per the inclusion criteria, patients must agree not to make any changes to their usual diet during the study, except as required for study-specific procedures (e.g., Bilitec).

Safety and Efficacy Assessments

Adverse Events (AE)

An AE is any untoward medical occurrence in a patient or clinical investigation subject administered a pharmaceutical product and which does not necessarily have to have a causal relationship with this treatment. An AE, therefore, can be any unfavorable and unintended sign (including an abnormal laboratory finding, for example), symptom, or disease temporally associated with the use of a medicinal product, whether or not considered related to the medicinal product.

An AE includes, but is not limited to, the following: Any unfavorable changes in general condition; Any clinically significant worsening of a pre-existing condition; Any intercurrent diseases and accidents; A procedure is not an AE, but the reason for a procedure may be an AE.

Causality Assessment

For all AEs, the Investigator must provide an assessment of causal relationship to study medication. The causality assessment must be recorded in the patient's source documentation and on the AE page of the patient's eCRF. Causal relationship must be assessed according to the following: Related: An AE where there is a reasonable possibility of a causal relationship between the event and the study medication; Unrelated: Any other AE.

Severity Assessment

The Investigator provides an assessment of the severity of each AE by recording a severity rating in the patient's source documentation and on the AE page of the patient's eCRF. Severity is to be assessed according to the following scale:

Mild: The AE was an annoyance to the patient but did not further hinder baseline functioning

Moderate: The AE caused the patient to experience some discomfort or some interference with normal activities but was not hazardous to health; prescription drug therapy may have been employed to treat the AE

Severe: The AE caused the patient to experience severe discomfort or severely limited or prevented normal activities and represented a definite hazard to health; prescription drug therapy and/or hospitalization may have been employed to treat the AE

A serious AE (SAE) is defined as any AE occurring at any dose that results in any of the following outcomes: Death; Life-threatening: the patient was at immediate risk of death from the reaction as it occurred (i.e., it does not include a reaction that hypothetically might have caused death had it occurred in a more severe form); Hospitalization or prolongation of an existing hospitalization; Persistent or significant disability or incapacity: a substantial disruption of a person's ability to conduct normal daily functions; Congenital anomaly or birth defect.

Important medical events: events that may not result in death, be life-threatening, or require hospitalization. Such an event may be considered serious when, based on appropriate medical judgment, it may jeopardize the subject and may require medical or surgical intervention to prevent 1 of the outcomes listed in this definition. Examples of such medical events include allergic bronchospasm requiring intensive treatment in an emergency department or at home, blood dyscrasias or convulsions that do not result in inpatient hospitalization, or the development of drug dependency or drug abuse.

Emergency room visits that do not result in admission to the hospital should be evaluated for 1 of the other serious outcomes (e.g., life-threatening, other serious [medically important] event).

Reporting Adverse Events and Serious Adverse Events and Pregnancy

Special Situation: Exposure to Study Drug During Pregnancy

A patient who reports pregnancy prior to study drug randomization must be withdrawn from the study. The pregnancy is recorded as a reason for screen failure. Since there has been no exposure to study drug, there is no need to notify Ironwood Drug Safety of the pregnancy. A patient who reports pregnancy after randomization, must discontinue study drug at once.

General Reporting Requirements

Adverse events are collected and recorded from the time the patient signs the ICF at the Screening Visit until 7 days after the EOT Visit. The study site makes contact with the patient via telephone 7 days following the EOT visit to collect information pertaining to ongoing AEs and information concerning new AEs. All AEs, regardless of the assumption of a causal relationship with study procedures or study medication, must be recorded in the patient's source documentation and subsequently on the appropriate AE page of the patient's eCRF. This record includes AEs the patient reports spontaneously, those observed by the Investigator, and those elicited by the Investigator in response to open-ended questions during the study, such as “Have you had any health problems since your last visit?”

For every AE, the Investigator must

Provide an assessment of the severity, causal relationship to the study medication, and seriousness of the event;

Document all actions taken with regard to the study medication (i.e., no action taken, treatment temporarily interrupted, or treatment discontinued); and

Detail any other treatment measures taken for the AE, including concomitant medications and/or procedures.

Pretreatment AEs are collected from the time the patient signs the ICF until the patient receives study medication. Pretreatment AEs are captured in the patient's source documentation, but are only entered for randomized patients on the AE page of the patient's eCRF.

Laboratory abnormalities and changes in vital signs, physical examination findings, and 12-lead ECG parameters should be considered AEs and reported on the AE page of the patient's eCRF if the Investigator considers them clinically significant and/or they necessitate intervention.

Any medical condition that is present when a patient is screened and does not worsen in severity and/or frequency should be reported as Medical History and not as an AE. However, if the condition does deteriorate in severity and/or frequency at any time during the study, it should be reported as an AE.

Appropriate remedial measures should be taken by the Investigator using his/her best medical judgment to treat the SAE. These measures and the patient's response to these measures should be recorded. All SAEs regardless of relationship to study medication, are to be followed by the Investigator until satisfactory resolution, the Investigator deems the SAE to be chronic or stable, or until the patient is lost to follow-up. Clinical, laboratory, and diagnostic measures should be employed by the Investigator as needed to adequately determine the etiology of the event.

Medical History

A complete medical history is performed as defined in the Schedule of Events (Table 2).

Physical Examination, Body Weight, and Height

A complete physical examination is performed as defined in the Schedule of Events (Table 2). The physical examination of each patient should include examination and assessment of the following:

General appearance Head, eyes, ears, nose, and throat

Cardiovascular system Neck

Respiratory system Musculoskeletal system

Abdomen/liver/spleen Nervous system

Lymph nodes Skin

Neurologic status Mental status

Breast, genitourinary, and rectal examinations are optional and may be performed at the discretion of the Investigator. Any new, clinically significant abnormal findings from the physical examination is reported as an AE.

Each patient's weight is recorded at every study visit; height is only recorded at the Screening Visit.

Electrocardiograms (ECG)

A 12-lead ECG is performed of Events (Table 2) and documented on the eCRF.

Electrocardiograms should be obtained after the patient has been supine for at least 5 minutes.

Vital Signs

Vital sign measurements are performed as defined in the Schedule of Events (Table 2) and documented on the eCRF. Vital sign measurements include oral temperature (° C.), respiratory rate, systolic and diastolic blood pressure (BP), and pulse. Respiratory rate, pulse, and BP readings are taken after the patient has been seated for at least 5 minutes.

Clinical Laboratory Determinations

Blood and urine samples for clinical laboratory tests are collected at the visits defined in the Schedule of Events (Table 2). The clinical laboratory evaluations include the clinical chemistry, hematology, coagulation, and urinalysis parameters are presented in Table 1.

TABLE 1
Clinical Laboratory Tests

| Clinical Chemistry | Hematology (CBC) | Complete Urinalysis |
|---|---|---|
| A1C | Hematocrit | pH and specific gravity |
| Albumin | Hemoglobin | Bilirubin |
| Alkaline phosphatase | Platelet count | Glucose |
| ALT | MPV | Ketones |
| AST | RBC count | Leukocytes |
| Bicarbonate | WBC count | Nitrites |
| BUN | WBC differential | Occult blood |
| Calcium | (% and absolute) | Protein |
| Chloride | Basophils | Urobilinogen |
| Total cholesterol | Eosinophils | |
| HDL cholesterol | Lymphocytes | |
| LDL cholesterol | Monocytes | |
| Creatinine | Neutrophils | |
| GGT | RBC indices | |
| Glucose | MCH | |
| Iron | MCHC | |
| LDH | MCV | |
| Magnesium | RDW | |
| Phosphorus | Coagulation | |
| Potassium | aPTT | |
| Sodium | PT | |
| Total bilirubin | | |
| Total protein | | |
| Triglycerides | | |
| Uric acid | | |

Abbreviations:
A1C = glycated hemoglobin;
ALT = alanine aminotransferase;
aPTT = activated partial thromboplastin time;
AST = aspartate aminotransferase;
BUN = blood urea nitrogen;
CBC = complete blood count;
GGT = gamma glutamyl transferase;
HDL = high-density lipoprotein;
LDH = lactate dehydrogenase;
LDL = low- density lipoprotein;
MCH = mean corpuscular hemoglobin;
MCHC = mean corpuscular hemoglobin concentration;
MCV = mean corpuscular volume;
MPV = mean platelet volume;
PT = prothrombin time;
RBC = red blood cell;
RDW = red cell distribution width;
WBC = white blood cell

A blood sample for serum pregnancy testing are collected from all female patients of childbearing potential (i.e., women who are not postmenopausal or who have not had a bilateral oophorectomy, hysterectomy, or tubal ligation) at the Screening and EOT Visits, and a urine sample is collected at the Randomization Visit (prior to dosing) and the Week 4 Visit.

These pregnancy test results must be negative for patient eligibility.

A urine screen for selected drugs of abuse (cocaine, barbiturates, amphetamines, opiates, benzodiazepines, and cannabinoids) and a serum alcohol screen are performed at the Screening Visit.

Esophagogastroduodenoscopy (EGD)

At all sites, an EGD is performed on all patients at the screening visit. An additional EGD is performed at the Week 8/EOT Visit in all patients who had an EGD during the Screening Period that demonstrated Grade C or D esophagitis (based on the Los Angeles classification of esophagitis; Table 4).

Bravo®

At all sites, all patients are to undergo 48 to 96 hours of pH testing with the Bravo® device (Given Imaging, Duluth Ga., USA). Bravo® pH monitoring is a capsule-based, patient-friendly test for identifying the presence of acid reflux. Bravo® pH monitoring device is a catheter-free, capsule-based pH monitoring device that is attached to a patient's esophagus. Information is collected over multiple days, which allows the doctor to evaluate reflux symptoms by determining the frequency and duration of acid flowing back up into the esophagus. The test is used to confirm if the patient's symptoms are caused by gastroesophageal reflux disease (GERD).

If for some reason 96 hours of testing is not possible, then approximately 48 hours of testing is acceptable. During the entire period of Bravo® pH monitoring (approximately 48-96 hours), patients record ingestion of anything other than water by depressing the ‘meal’ button on the Bravo® device upon the beginning and completion of the event. Patients are to also record any periods during which they are lying down by depressing the ‘supine’ button on the Bravo device at the beginning and upon completion of the supine period. In addition, for the entire 48-96 hour Bravo® pH monitoring period, patients are also to complete a paper diary, recording at a minimum all instances of meals, snacks, drinks, and/or resting in the supine position. All patients are to return to the site with their Bravo pH monitor and patient diary after approximately 96 hours (or 48 hours where applicable) to return the recording device.

Bilitec®

At certain participating sites, selected patients who are screened with EGD and Bravo testing also receive insertion of a Bilitec device during the same procedure. The patient selection depend on Investigator selection and patient consent. All such patients return to the site approximately 24 hours after the procedure for removal of the device. All patients participating in the Bilitec® testing also have to follow very specific procedures including a “white diet” during this 24 hour period.

Efficacy Assessments

The daily patient assessments used to determine the key efficacy parameters are the daily assessment of heartburn symptoms (assessed on a 0-to-5 ordinal severity scale) and regurgitation symptoms (assessed on a 0-to-4 ordinal frequency scale) obtained from the mRESQ-eD. Additional assessments also are used to determine the other efficacy parameters.

Symptom Severity Assessment

Daily Assessments

During the Pretreatment and Treatment Periods, patients are to enter information into their eDiary at approximately the same time each day.
GERD symptoms (mRESQ-eD) completed once daily before going to bed each night.

The following items are assessed on a 0-5 severity scale: 0=Did not have, 1=Very mild, 2=Mild, 3=Moderate, 4=Moderately severe, 5=Severe

Heartburn; Burning feeling behind breastbone or in the center of the upper stomach; Pain behind the breastbone or in the center of the upper stomach; Difficulty swallowing; Hoarseness; Cough

The following items are assessed on a 0-4 frequency scale: 0=Never, 1=Rarely, 2=Sometimes, 3=Often, 4=Very often

Regurgitation (liquid or food) moving upwards toward your throat or mouth; An acid or bitter taste in the mouth; Burping; Coughing; Dyspepsia symptoms (Daily Dyspepsia Symptoms, Appendix 2) completed once daily before going to bed each night.

All items are assessed on a 0-to-10 numerical rating scale (NRS) with 0=not having the symptom and 10=having the worst possible level of the symptom
Worst nausea (feeling like you might throw up)
Worst stomach fullness after you finished eating
Difficulty you had finishing your meals because you felt full too quickly
Worst abdominal pain. Assessment of sleep (Daily Assessment of Sleep, Appendix 3) completed once daily upon getting up each morning (5:00 a.m. to 12:00 p.m.)
Last night, did you wake up during the night after falling asleep? Y/N
[If yes], how many times did you wake up last night after falling asleep?
How long did you sleep last night? Do not count any time you lay in bed, but did not sleep.
Please rate the overall quality of your sleep last night. 1=very poor, 2=poor, 3=fair, 4=good, 5=very good

Use of Per-Protocol Rescue Medicine (Antacids)

Weekly Assessments

The following information are entered into the eDiary each week at about the same time as the evening daily assessment (see Appendix 6):

Degree of Relief Questions

all items are assessed on a 7-point balanced ordinal scale: 1=Significantly relieved, 2=Moderately relieved, 3=Somewhat relieved, 4=Unchanged, 5=Somewhat worse, 6=Moderately worse, 7=Significantly worse
How would you rate your heartburn (a burning sensation in your chest, behind the breastbone) over the past 7 days?
How would you rate your regurgitation (the feeling of stomach contents, either liquid or food, moving upwards to your throat or mouth) over the past 7 days?
Compared to before you started this study, how would you rate your overall GERD symptoms over the past 7 days?

Global Treatment Satisfaction Assessment

the following items are assessed on a 5-point ordinal scale: 1=Very dissatisfied, 2=Dissatisfied, 3=Neither satisfied nor dissatisfied, 4=Satisfied, 5=Very satisfied
How would you rate your satisfaction with the study treatment?

Bothersomeness Assessment.

the following items are assessed on a 5-point ordinal scale: 1=Not at all, 2=A little bit, 3=A moderate amount, 4=A great deal, 5=An extreme amount
How much were you bothered by heartburn (a burning sensation in your chest, behind the breastbone) over the past week?
How much were you bothered by regurgitation (the feeling of stomach contents, either liquid or food, moving upwards to your throat or mouth) over the past week?

Weekly items assessing symptom bothersomeness and symptom relief is used to explore responder definitions and treatment benefit analysis.

GSRS-Self

The Gastrointestinal Symptoms Rating Scale (GSRS)-Self is a self-administered 15-item questionnaire that uses a 7-point Likert scale for discomfort:

1=None; 2=Minor; 3=Mild; 4=Moderate; 5=Moderately severe; 6=Severe; 7=Very severe

The items can then be grouped into 5 domains: abdominal pain (3 items), reflux syndrome (2 items), indigestion (4 items), diarrhea (3 items), and constipation (3 items). Patients are to complete the GSRS-Self at the Randomization Visit, the Week 4 Visit, and at the EOT Visit; responses are recorded in an electronic diary via a portable PDA device.

The Questions are:

Have you been bothered by PAIN OR DISCOMFORT IN YOUR UPPER ABDOMEN OR THE PIT OF YOUR STOMACH during the past week?

Have you been bothered by HEARTBURN during the past week? (By heartburn we mean an unpleasant stinging or burning sensation in the chest.)

Have you been bothered by ACID REFLUX during the past week? (By acid reflux we mean the sensation of regurgitating small quantities of acid or flow of sour or bitter fluid from the stomach up to the throat.)

Have you been bothered by HUNGER PAINS in the stomach during the past week? (This hollow feeling in the stomach is associated with the need to eat between meals.)

Have you been bothered by NAUSEA during the past week? (By nausea we mean a feeling of wanting to throw up or vomit.)

Have you been bothered by RUMBLING in your stomach during the past week? (Rumbling refers to vibrations or noise in the stomach.)

Has your stomach felt BLOATED during the past week? (Feeling bloated refers to swelling often associated with a sensation of gas or air in the stomach.)

Has your stomach felt BLOATED during the past week? (Feeling bloated refers to swelling often associated with a sensation of gas or air in the stomach.)

Have you been bothered by PASSING GAS OR FLATUS during the past week? (Passing gas or flatus refers to the need to release air or gas from the bowel, often associated with easing a bloated feeling.)

Have you been bothered by CONSTIPATION during the past week? (Constipation refers to a reduced ability to empty the bowels.)

Have you been bothered by DIARRHEA during the past week? (Diarrhea refers to a too frequent emptying of the bowels.)

Have you been bothered by LOOSE STOOLS during the past week? (If your stools (motions) have been alternately hard and loose, this question only refers to the extent you have been bothered by the stools being loose.)

Have you been bothered by HARD STOOLS during the past week? (If your stools (motions) have been alternately hard and loose, this question only refers to the extent you have been bothered by the stools being hard.)

Have you been bothered by an URGENT NEED TO HAVE A BOWEL MOVEMENT during the past week? (This urgent need to go to the toilet is often associated with a feeling that you are not in full control.)

When going to the toilet during the past week, have you had the SENSATION OF NOT COMPLETELY EMPTYING THE BOWELS? (This feeling of incomplete emptying means that you still feel a need to pass more stool despite having exerted yourself to do so.)

QIDS-SR-16

The Quick Inventory of Depressive Symptomatology (QIDS)-SR-16 is a self-completed questionnaire designed to assess the severity of 9 clinically defined symptoms of depression on a scale from 0 (no symptom impact) to 3 (severe impact). Rush A J et al. The 16-Item Quick Inventory of Depressive Symptomatology (QIDS), clinician rating (QIDS-C), and self-report (QIDS-SR): a psychometric evaluation in patients with chronic major depression. Biol Psychiatry 2003; 54(5):573-83. The tool can be used to screen for depression or as a measure of symptom severity. Patients are to complete the QIDS-SR-16 at the Randomization Visit.

SF-12V2 Health Survey

The SF-12V2 is a widely used generic measure of health status and measures 8 concepts of health: physical functioning, role limitations due to physical health problems, bodily pain, general health, vitality (energy/fatigue), social functioning, role limitations due to emotional problems, and mental health (psychological distress and psychological well-being). These 8 scales are aggregated into 2 summary measures: the physical component and mental component summary scores. Patients are to complete the SF-12V2 at the Randomization Visit, the Week 4 Visit, and at the EOT Visit.

EQ-5D-3L

The EuroQol (EQ)-5D-3L is a generic measure of health widely used in Europe. The EuroQol Group. EuroQol—a new facility for the measurement of health-related quality of life. Health Policy 1990; 16(3):199-208. The first component consists of 5 questions assessing the following dimensions: mobility, self-care, usual activities, pain/discomfort, and anxiety/depression. Responses to the 5 questions define a health state for which a utility index can be derived from published algorithms. Shaw J W et. Al, Med Care 2005; 43(3):203-20. The second component of the EQ-5D is a visual analogue scale, asking patients to rate their health from 0 to 100 (0 represents worst imaginable health state and 100 represents best imaginable health state). Patients complete the EQ-5D-3L at the Randomization Visit, the Week 4 Visit, and at the EOT Visit; responses are recorded in an electronic diary via a portable PDA device.

End-of-Treatment Question

Patients are asked a single item at their final visit (EOT), asking about the difficulty of swallowing the treatment medication. The item asks “How difficult were the tablets to swallow?” and be rated on a 4 point ordinal scale:

1=Not at all difficult 2=A little difficult 3=Moderately difficult 4=Extremely difficult

mRESQ Cognitive Debriefing Interviews

Cognitive debriefing interviews are conducted to support the modified mRESQ as fit for purpose in the context of a clinical trial. The original RESQ-eD was modified including instructions, removal of redundant items, and inclusion of alternate response scales for selected symptoms. Guidance on instrument development emphasizes the need for adequate empirical evidence in the patient population targeted for enrollment in the clinical trials to support content validity for the desired claim. To that end, a sample of patients at selected sites are asked to participate in an optional symptom diary interview, either via phone or face to face.

Approximately 30 subjects are interviewed from participating sites; 5 screen failures, 5 pre-treatment and 20 active treatment subjects are interviewed. During the one-time interview, patients are asked to respond to open-ended questions intended to assess content validity of the mRESQ-eD instrument.

TABLE 2
Schedule of Events

| | Visit Days | | | | | | |
|---|---|---|---|---|---|---|---|
| | Screening | | | | | | |
| | Period | Pretreatment | | | | | |
| | (Up to 4 | Period | | | | | |
| | weeks)^{w} | (2 weeks) | Treatment Period (8 weeks) | | | | |
| | Screening | Pretreatment | | | | Week 8/End- | |
| | Visit | Visit | Randomization | Week 2 | Week 4 | of-treatment | Follow-up |
| | (Day −49 to | (Day −21 to | Visit | Visit | Visit | Visit (Day | EOT + |
| | Day −15) | Day −1) | (Day 1) | (Day 15 ± 3) | (Day 29 ± 3) | 57 + 3) | 7 days |
| | Visit Numbers | | | | | | |
| | Visit 1 | Visit 2 | Visit 3 | Visit 4 | Visit 5 | Visit 6 | |
| Study | | | | | | | |
| Procedure 1 | | | | | | | |
| Drug and | X | | | | | | |
| Alcohol | | | | | | | |
| Screen (m) | | | | | | | |
| AE | X | X | X | X | X | X | |
| Evaluations (n) | | | | | | | |
| Rescue | | X | X | X | X | | |
| Medicine | | | | | | | |
| Dispensed (o) | | | | | | | |
| PDA | | X | | | | | |
| Training | | | | | | | |
| and | | | | | | | |
| Dispensation | | | | | | | |
| Saliva | X | X | X | | X | X | |
| Collection (p) | | | | | | | |
| eDiary (q) | | X | X | X | X | X | |
| Weekly | | X | X | X | X | X | |
| Symptom and | | | | | | | |
| Treatment | | | | | | | |
| Assessments (r) | | | | | | | |
| GSRS- | | | X | | X | X | |
| Self | | | | | | | |
| QIDS-SR-16 | | | X | | | | |
| SF-12V2 | | | X | | X | X | |
| EQ-5D- | | | X | | X | X | |
| 3L | | | | | | | |
| Randomization | | | X | | | | |
| Study | | | X | X | X | | |
| Medication | | | | | | | |
| Dispensed (s) | | | | | | | |
| Study | | | | X | X | X | |
| Medication | | | | | | | |
| Return (t) | | | | | | | |
| PDA Return | | | | | | X | |
| mRESQ | | X (x) | | | X (x) | | |
| Debrief | | | | | | | |
| Interview (x) | | | | | | | |
| End-of- | | | | | | X | |
| treatment | | | | | | | |
| Question (u) | | | | | | | |
| Safely | | | | | | | X |
| Follow-up | | | | | | | |
| Call (v) | | | | | | | |

Abbreviations: AE = adverse event; BP = blood pressure; CBC = complete blood count; ECG = electrocardiogram; EGD = esophagogastroduodenoscopy;
EOT = End-of-treatment; EQ = EuroQol; GSRS = Gastrointestinal Symptoms Rating Scale; H2RA = histamine-2 receptor antagonist; HEENT = head, eyes, ears, nose, and throat; ICF = informed consent form; IWRS = interactive web response system; PDA = personal digital assistant; PPI = proton pump inhibitor;
QID = Quick Inventory of Depressive Symptomology; mRESQ-eD = modified Reflux Symptom Questionnaire Electronic Diary; SAE = serious adverse event;
SF = short form
^{a.} Site personnel interacts with IWRS to register the patient visit and transition the patient to the next appropriate study period.
^{b.} A physical examination should include the following assessments: general appearance; HEENT; neck; cardiovascular; respiratory; abdomen/liver/spleen; musculoskeletal; lymph nodes; skin; neurologic; neryous system, and mental status. Breast, genitourinary, and rectal examinations are optional and may be performed at the discretion of the Investigator.
^{c.} Height I measured only at the Screening Visit.
^{d.} During the Screening Period, patients begin to washout H2RAs and antacids. H2RAs should be stopped 5 calendar days prior to the EGD and Bravo pH monitoring and antacids should be stopped 1 calendar day prior to the EGD and Bravo pH monitoring. Patients may resume antacid use upon completion of the Bravo testing, but must refrain from H2RA use for the remainder of the study. During the Pretreatment Period, patients refrain from using any anti- reflux medications, antacids, and H2RAs, except for the antacids that are provided as rescue medicine). Patients continue to use their current PPI during the Pretreatment Period.
^{e.} At selected sites, selected patients who are screened for the study with EGD and Bravo testing also to be given the option of having a Bilitec monitor inserted during the same procedure. These patients are to return 24 hours later for removal of the probe. The results of the Bilitec test do not affect qualification for enrollment. The Bilitec device and Bravo recorder internal clocks must be synchronized, and both devices should be activated concurrently; however, if it is not possible to simultaneously activate both devices, the Bravo recorder should be activated first, with the Bilitec device activated immediately (within 5 minutes) after the Bravo pH recorder activation.
^{f.} All patients are required to undergo an EGD during the Screening Period. There must be a minimum of 7 days between the EGD and the start of the Pretreatment Period. An EGD is performed at the Week 8/EOT Visit in all patients who have Grade C or D esophagitis (based on the Los Angeles classification of esophagitis) on the EGD obtained during the Screening Period.
^{g.} Approximately 48 to 96 hours ot pH testing with the Bravo device. It tor some reason 96 hours of testing is not possible, then approximately 48 hours of testing is acceptable. During the entire period of Bravo pH monitoring (approximately 48-96 hours), patients record ingestion of all meals, snacks, and drinks by depressing the ‘meal’ button on the Bravo recorder upon the beginning and completion of the meal, snack, or drink. Patients also record any periods during which they are lying down by depressing the ‘supine’ button on the Bravo recorder at the beginning and upon completion of the supine period. In addition, for the entire 48-96-hour Bravo pH monitoring period, patients also complete a paper diary, recording at a minimum, all instances of meals, snacks, drinks, and/or resting in the supine position.
^{h.} Vital sign measurements include oral temperature (° C.), respiratory rate, systolic and diastolic BP, and pulse. Respiratory rate, BP, and pulse measurements must be obtained after the patient has been seated for at least 5 minutes.
^{i.} 12-lead ECGs should be obtained after the patient has been supine tor at least 5 minutes.
^{j.} Prior medications are collected at the Screening Visit as follows: all medicines taken by the patient during the 30 days before the Screening Visit, most recent use of an H2RA, and most recent use of an antacid.
^{k.} Clinical laboratory tests include clinical chemistry, hematology (CBC), coagulation, and urinalysis. If the triglyceride value exceeds the protocol-specified criteria and the patient was not under fasted conditions, the patient may return to completed a fasted lipid panel.
^{l.} For all female patients of childbearing potential, a negative serum pregnancy test must be documented at the Screening Visit, and a negative urine pregnancy test must be documented at the Randomization Visit (before dosing) for the patient to be randomized into the study. A urine pregnancy test is obtained at the Week 4 Visit; serum pregnancy test is conducted at the EOT Visit.
(m) Patients must undergo a urine drug screen for selected drugs of abuse (cocaine, barbiturates, amphetamines, opiates, benzodiazepines, and cannabinoids) and a serum alcohol screen at the Screening Visit.
(n) All AEs are captured from the time the patient signs the ICF through the EOT Visit.
(o) Rescue medicine is supplied to patients at the Pretreatment Visit, and if needed, at subsequent visits.
(p) At each of the indicated visits, patients provide approximately 1 mL of saliva for future use (bile acids are quantified in the saliva, and the quantity of bile acid may be used to define potential responders to IW-3718). At the time of saliva collection, study site staff is to collect the following information: time of day, time of patient's last meal, and time of last study medication administration. During the Randomization Visit, study site staff is to collect the saliva sample immediately following the light snack but prior to study drug administration.
(q) The eDiary is dispensed at the Pretreatment Visit and patients must complete 5 days each week during the 14 calendar days before the Treatment Period to be eligible for randomization. Patients should bring their eDiary to each visit. The eDiary collects daily PPI administration, rescue medication use, mRESQ-eD (daily), Daily Assessment of Sleep (daily), Daily Dyspepsia Symptoms (daily).
(r) Symptom bothersomeness and relief items (weekly); Treatment satisfaction item (weekly).
(s) The first dose of study medication is administered in the clinic with liquid and a snack at the Randomization Visit. At all other visits, patients take their study medication prior to arriving at the clinic, but are to be dispensed additional doses needed until the next study visit.
(t) Treatment compliance with study drug is assessed based on return of unused tablets.
(u) All patients are asked an EOT question regarding the difficulty of swallowing the IW-3718 or placebo tablets.
(v) Study site is to contact each patient via telephone 7 days after the EOT Visit to collect information pertaining to ongoing AEs/SAEs and information concerning any new AEs/SAEs since the EOT Visit
^{w}Ironwood may grant a one-week extension of the screening period window if needed for logistical delays (e.g., subject travel, scheduling issues, delays in test results, equipment malfunction, etc.). Approval should be requested from Ironwood prior to each extension.
(x) Patients participating in the optional mRESQ-eD Cognitive Debriefing interviews are assigned to one of three interview groups. Interviews take place at different timepoints depending on the patient's group assignment.

Screening Period (Day −49 to Day −15)

Screening Visit (Visit 1) Procedures

Signing of ICF (informed consent form); Register visit in IWRS; Review of inclusion and exclusion criteria; Demographics; Medical history; Physical examination; Body weight and height; Begin H2RA and antacid washout (for five calendar days before the EGD and Bravo pH monitoring [H2RA] and 1 calendar day before the EGD and Bravo pH monitoring [antacids])

EGD

Approximately 48 to 96 hours of pH testing with a Bravo® device (if 96 hours of testing is not possible, then approximately 48 hours of testing is acceptable); Optional 24 hours of measurements of bilirubin using the Bilitec device (for patients undergoing the Bilitec procedure—device removed after 24 hours)

Seated Vital Signs

12-Lead ECG

Prior medications (all medicines taken during the 30 days before the Screening Visit, most recent use of an H2RA, and most recent use of an antacid)

Collection of blood and urine samples for clinical laboratory test, including:

Clinical Chemistry

Hematology (complete blood count [CBC]); Coagulation; Urinalysis; Serum pregnancy test for all female patients of childbearing potential (must be confirmed negative); AE evaluation (throughout the Screening Period); Saliva collection

Extension of Screening Period: one-week extension of the screening period window may be granted if needed for logistical delays (e.g., subject travel, scheduling issues, delays in test results, equipment malfunction, etc.).

If an extension of greater than one week is needed for logistical delays, subjects are considered screen failures and need to re-screen. If these subjects had already completed the EGD/Bravo (and Bilitec, if applicable) assessments during the original screening period, they do not need to repeat these tests if they enter Pre-Treatment within 35 days of the assessments. All other study assessments should be repeated during re-screening. Pretreatment Period (Day −21 to Day −1).

Pretreatment Visit (Visit 2) Procedures

Register visit in IWRS; Review of inclusion and exclusion criteria; Body weight; Seated vital signs; Prior and concomitant medications; AE evaluation (throughout the Pretreatment Period); Antacid rescue medicine dispensation; PDA training; PDA is dispensed (for recording daily and weekly evaluations throughout the Pretreatment and Treatment Periods in the eDiary); Saliva collection

A subset of patients participating in the optional Cognitive Debriefing Interview at selected sites is to undergo the interview at this visit.

Treatment Period (Day 1 to Day 57)

For all visits during the Treatment Period, patients are to take their PPI and study medication (except for the Randomization Visit) prior to reporting to the study site.

Randomization Visit (Visit 3) Procedures

Register visit in IWRS; Review of inclusion and exclusion criteria; Body weight; Seated vital signs; 12-lead ECG; Concomitant medications.

Collection of blood and urine samples for clinical laboratory tests, including: Clinical chemistry; Hematology (CBC); Coagulation; Urinalysis; Urine pregnancy test for all females of childbearing potential (must be confirmed negative prior to dosing); AE evaluation (throughout the Treatment Period); Antacid rescue medicine dispensation (if needed); Saliva collection; Collect PDA and review eDiary; GSRS-Self; QIDS-SR-16; SF-12V2; EQ-5D-3L; Randomization; Study medication dispensed.

Study medication administration (first dose of study medication taken in clinic with liquid and a snack. Patients should take their second dose that evening, with liquid, immediately upon completion of dinner and ensure that at least 8 hours have elapsed since the first dose in clinic).

Week 2 Visit (Visit 4) Procedures

Register visit in IWRS; Body weight; Seated vital; signs; Concomitant medications; AE evaluation (throughout the Treatment Period); Antacid rescue medicine dispensation (if needed); Collect PDA and review eDiary; Return of all unused study medication; Additional study medication dispensed

Week 4 (Visit 5) Procedures

Register visit in IWRS; Body weight; Seated vital signs; Concomitant medications; Collection of blood and urine samples for clinical laboratory tests, including: Clinical chemistry; Hematology (CBC); Coagulation; Urinalysis; Urine pregnancy test for all females of childbearing potential; AE evaluation (throughout the Treatment Period); Antacid rescue medicine dispensation (if needed); Saliva collection; Collect PDA and review eDiary; GSRS-Self; SF-12V2; EQ-5D-3L; Study medication dispensed; Return of all unused study medication

A subset of patients participating in the optional Cognitive Debriefing Interview at selected sites to undergo the interview at this visit.

Week 8/End-of-treatment (EOT) (Visit 6) Procedures

Register visit in IWRS; Physical examination; Body weight; EGD; Seated vital signs; 12-lead ECG; Concomitant medications; Collection of blood and urine samples for clinical laboratory tests, including: Clinical chemistry; Hematology (CBC); Coagulation; Urinalysis; Serum pregnancy test for all female patients of childbearing potential; AE evaluation (throughout the Treatment Period); Saliva collection; Review eDiary; GSRS-Self; SF-12V2; EQ-5D-3L; Return of all unused study; medication; Return PDA device; End of treatment question; Follow-up.

The study site is to contact all patients via telephone 7 days following the EOT Visit to collect information regarding ongoing AEs and/or SAEs and any new AEs and/or SAEs since the EOT Visit.

Patients who discontinue from the study for any reason should complete the assessments required at the EOT Visit at the time of their discontinuation.

Statistical Methods

Analysis Populations

The Screened Population consists of all patients who consented to participate in the study.

The modified Intent-to-Treat (mITT) Population consists of all randomized patients who received at least 1 dose of study treatment.

The Per-Protocol Population (PP) is defined as those patients in the mITT Population who have a minimum of 6 weeks of eDiary data for the heartburn severity and regurgitation frequency scores and >80% compliance with study treatment for the available Treatment Period days.

The Safety Population is defined as all randomized patients who received at least 1 dose of study treatment.

General Methods

Continuous variables are summarized using descriptive statistics (n, mean, standard deviation, median, and range). Categorical variables are summarized using the subject count and proportions of patients in each category. Unless otherwise specified, all confidence intervals are two-sided and with a confidence level of 95%. Due to the exploratory nature of the trial, no adjustments are made for multiplicity. If not otherwise specified, the baseline value is defined as the last non-missing value measured before administration of study treatment on Day 1. All statistical analyses are performed using SAS® Version 9.3 (or later) for Windows.

Patient Disposition, Demographics, and Baseline Characteristics

Efficacy Analyses

Table 3 provides the analysis time windows allowed for the efficacy analyses in the Pretreatment and Treatment Periods.

TABLE 3
Analysis Time Windows for Efficacy Analysis

| | Analysis | | |
|---|---|---|---|
| Period | Week | Begins^{a} | Ends^{a} |
| Pretreatment | Week −2 | Day −14 | Day −8 |
| (Baseline^{b}) | Week −1 | Day −7 | Day −1 (Day before |
| Treatment | | | first dose) |
| | Week 1 | Day 1 (Day of first dose) | Day 7 |
| | Week 2 | Day 8 | Day 14 |
| | Week 3 | Day 15 | Day 21 |
| | Week 4 | Day 22 | Day 28 |
| | Week 5 | Day 29 | Day 35 |
| | Week 6 | Day 36 | Day 42 |
| | Week 7 | Day 43 | Day 49 |
| | Week 8 | Day 50 | Day 56 |

^{a}Relative to the date of first dose (Day 1).
^{b}Baseline values for efficacy parameters are derived from the daily eDiary and eCRF data collected in the Pretreatment Period Week −2 and Week −1.

Primary and Secondary Efficacy Parameters

The primary efficacy parameter is the percent change from baseline (i.e., Pretreatment) in weekly heartburn severity score (WHSS) at Week 8. Daily heartburn severity score (DHSS) is defined as the maximum of the 3 items measuring heartburn from a particular day collected with the mRESQ-eD instrument; WHSS is defined as the average of available DHSS in a particular week.

The secondary efficacy parameters include the following: Percent change from baseline in WHSS at Week 4; Change from baseline in WHSS at (a) Week 4 and (b) Week 8; Proportion of patients who are overall heartburn responders; Weekly Heartburn Responder: patient with a decrease from baseline of ≥30% in WHSS; Overall heartburn responder: patient who is a weekly heartburn responder for at least 1 of the final 2 weeks and for ≥50% of the treatment weeks; Proportion of patients with a DHSS of no more than very mild (≤1) on any day during (a) Week 4 and (b) Week 8; The number of days per week where DHSS was no more than very mild (≤1) at (a) Week 4 and (b) Week 8.

Change from baseline in each mRESQ-eD item by week.

Proportion of Heartburn Free Days in Week 4 and Week 8

DHSS is defined as the maximum of the 3 items in the heartburn assessments of a particular day, collected with the mRESQ-eD instrument. Further, WHSS is defined as the average of DHSS in a particular week.

Heartburn-free days is calculated by 2 approaches. The first employs the single heartburn item in the heartburn domain (mRESQ-eD: Heartburn), similar to the heartburn rating scale(s) used in the PPI literature. The second employs all 3 items in the heartburn domain (mRESQ-eD: Heartburn; Burning feeling behind the breastbone or in the center of the upper stomach; Pain behind the breastbone or in the center of the upper stomach). In the first approach, the heartburn item needs to be assessed at 0 (Did not have) while in the second approach all 3 heartburn domain items need to be assessed at 0 (Did not have) For the analysis of continuous parameters (e.g., absolute change from baseline and percent change from baseline), descriptive statistics (patient number [n], mean, standard deviation, median, and range) is presented for each treatment group. Each IW-3718 group is compared with the placebo group using an analysis of covariance (ANCOVA) model with treatment group, and esophagitis stratum as fixed effect terms and the corresponding baseline efficacy parameter value as a covariate. Least-squares means (LSMs) for each treatment group, a linear contrast among the LSMs to test the overall ordinal dose response, LSMs differences between each IW-3718 group and the placebo group and their corresponding confidence intervals, and p-values for the pairwise comparisons versus placebo are presented. The treatment by esophagitis stratum interaction term is to be explored to evaluate whether quantitative or qualitative interaction(s) are present, and treatment comparisons within each stratum is conducted, as warranted The cumulative distribution function (CDF) of change from baseline for key efficacy parameters is plotted by treatment group. To aid in the interpretation of the graphical representation of the CDF across treatments, a two-sample Kolmogorov-Smirnov test is conducted. Hollander M, Wolfe D A. Chapter 10: Tests Designed to Detect Broad Alternatives. In: Nonparametric Statistical Methods. New York, N.Y.: John Wiley & Sons; 1973. p. 219. A p-value≤0.05 indicates that the 2 treatment samples are unlikely to have been drawn from the same continuous distribution by chance alone.

For the analysis of responder parameters (i.e., responder vs. non-responder), the counts and proportion of responders are calculated for each treatment group. The proportions of responders between each IW-3718 group and the placebo group area compared using a Cochran-Mantel-Haenszel (CMH) test controlling for esophagitis stratum. The CMH test is the primary analysis for responder parameters. The difference in the proportion of responders between each IW-3718 group and the placebo group as well as the CMH estimates of the odds ratio (IW-3718 over placebo) and the corresponding 95% confidence intervals are presented.

Other Efficacy Parameters Include the Following:

Percent change from baseline in weekly regurgitation frequency scores (WRFS) to (a) Week 4 and (b) Week 8 in patients with a mean baseline regurgitation frequency of ≥2 (sometimes).

Change from baseline in WRFS to (a) Week 4 and (b) Week 8

Proportion of patients who are overall regurgitation responders (of patients with a baseline regurgitation frequency of ≥2 [sometimes])

Weekly regurgitation responder: patient with a decrease from baseline of ≥30% in WRFS

Overall regurgitation responder: patient who is a weekly responder for at least 1 of the final 2 weeks and for ≥50% of the treatment weeks

Proportion of patients with regurgitation frequency of no more than rarely (daily regurgitation frequency score [DRFS]≤1) on any day during (a) Week 4 and (b) Week 8

Change from baseline to (a) Week 4 and (b) Week 8 in the number of days per week where DRFS was no more than rarely (≤1)

Change from baseline to Week 8 in weekly means for each of the daily symptom assessments

DRFS is the maximum of the 2 items in the regurgitation domain scores of a particular day, collected with the mRESQ-eD instrument. Similarly, WRFS is defined as the average of DRFS in a particular week. A DRFS of zero is considered Regurgitation Free for a particular day.

Weekly means for daily sleep assessments (number of awakenings, hours of sleep, sleep quality)

Weekly means for weekly symptom bothersomeness assessments, symptom relief assessments, and treatment satisfaction assessment

Exploratory non-efficacy parameters and analysis: Summarize patient baseline disease characteristics and key efficacy differences for patients with or without Bilitec monitoring performed during the Screening Period;

Compare patient baseline disease characteristics and key efficacy parameters between bile acid positive versus bile acid negative patients (on the subgroup of patients with Bilitec monitoring during the Screening Period).

Assess sensitivity and specificity (i.e., receiver operating characteristic [ROC] curve) of bile acid levels measured in saliva as a test for bile acid pathophysiology with Bilitec test findings as the reference standard (on the subgroup of patients with Bilitec monitoring during the Screening Period)

Utilize bile acid levels in saliva as a predictor of response to treatment

For analysis of continuous parameters, descriptive statistics (n, mean, standard deviation, median, and range) is presented for each treatment group. Each IW-3718 group is compared with the placebo group using an ANCOVA model with treatment group and esophagitis stratum as fixed effect terms and corresponding baseline efficacy parameter value as a covariate.

Safety Analyses

All safety parameters are analyzed with descriptive statistics. Safety analyses are performed on the Safety Population. The safety parameters include AEs, treatment-emergent AEs (TEAEs), clinical laboratory evaluations, vital signs, ECGs, and physical examination. For each safety parameter, the last non-missing assessment made before randomization is used as the baseline for all analyses of that safety parameter.

Adverse Events

Adverse event verbatim terms are coded using the most current version of Medical Dictionary for Regulatory Activities (MedDRA) available at the start of the study. An AE (classified by preferred term) is considered a TEAE if the AE onset date was after initial study medication administration and within 1 day of the last dose of study medication. The number and percentage of patients reporting TEAEs are tabulated by system organ class (SOC), preferred term, and treatment group. The number and percentage of patients reporting TEAEs are also tabulated by SOC, preferred term, severity, and treatment group. Listings are provided for deaths (if any), severe AEs, drug-related AEs, SAEs, and AEs leading to study discontinuation.

If a patient has more than 1 TEAE coded to the same preferred term, the patient is counted only once for that preferred term. For the analysis of TEAEs by severity, the patient's highest severity TEAE within a preferred term is used.

ECGs, Vital Signs, and Clinical Laboratory Tests

Descriptive statistics is calculated on ECGs, vital signs, and clinical laboratory test results at each assessment time point, by treatment group. The change from baseline at each post-baseline time point is also summarized by treatment group.

Determination of Sample Size

The sample size per arm was determined by estimating the overall power of a linear trend test in a one-way design that included placebo and all the active treatment arms (500 mg BID, 1000 mg BID, and 1500 mg BID of IW-3718). The efficacy endpoint of interest is assessed with an 11-point [0 to 10] NRS, where 0=none and 10=very severe. A study with 58 patients per arm (the expected number of patients at Week 8 given an enrollment of 260 patients) has statistical power of at least 80% for the overall trend test (i.e., linear contrast) where the highest active treatment arm reflects 110% of the previously observed treatment difference and the lowest 2 active treatment arms reflect 55% of the same (two-sided, α=0.05). At the proposed sample size, a subsequent pairwise comparison between placebo and an active treatment arm reflecting the previously observed treatment difference has 80% statistical power (two-sided, α=0.05).

mRESQ-eD requires a patient to answer the following questions since waking up.

Over the past 24 hours, how would you rate the severity of your [insert symptom]?

0=Did Not Have, 1=Very Mild, 2=Mild, 3=Moderate, 4=Moderately Severe, 5=Severe

1. Heartburn; 2. Burning feeling behind the breastbone or in the center of the upper stomach; 3. Pain behind the breastbone or in the center of the upper stomach; 4. Difficulty swallowing; 5. Hoarseness; 6. Coughing

Over the past 24 hours, how often did you experience [insert symptom]—0=Never, 1=Rarely, 2=Sometimes, 3=Often, 4=Very often

Regurgitation (liquid or food moving upwards towards your throat or mouth); An acid or bitter taste in the mouth; Burping; Cough

DAILY DYSPEPSIA SYMPTOMS are to be completed by a patient before going to bed each night. All items are assessed on a 0-to-10 numerical rating scale [NRS] with anchors of 0=not having the symptom and 10=having the worst possible symptom.

Over the past 24 hours, rate your worst nausea (feeling like you might throw up). NRS anchors: 0=No nausea, 10=Worst possible nausea

Over the past 24 hours, rate your worst stomach fullness after you finished eating. NRS anchors: 0=No stomach fullness, 10=Worst possible stomach fullness

Over the past 24 hours, how would you rate the difficulty you had finishing your meals because you felt full too quickly

NRS anchors: 0=No difficulty, 10=Worst possible difficulty

Over the past 24 hours, how would you rate your worst abdominal pain? NRS anchors: 0=No pain, 10=Worst possible pain

DAILY ASSESSMENT OF SLEEP is to be completed by a patient each morning upon waking.

Awakenings

Last night, did you wake up during the night after falling asleep? [Yes, No]

[If yes], how many times did you wake up last night after falling asleep? [Enter number of times]

Sleep Time

How long did you sleep last night? Do not count any time you lay in bed, but did not sleep. [Enter hours, enter minutes]

Overall Sleep Quality

Please rate the overall quality of your sleep last night.

1=Very poor 2=Poor 3=Fair 4=Good 5=Very good

Prohibited Medications

All medicine listed in the sections below are excluded during the Screening, Pretreatment, Randomization, and Treatment Periods. A 1-day washout means that the particular medicine is not allowed during the calendar day before the EGD and Bravo pH monitoring; a 5-day washout means that the particular medicine is not allowed during the 5 calendar days before the EGD and Bravo pH monitoring; a 14-day washout means that the particular medicine is not allowed during the 14 calendar days before the Pretreatment Visit.

Patients should be on a stable dose of all concomitant medications at the time of the Screening Visit and should intend to maintain their usual medication regimen throughout the study.

Changes in concomitant medication regimens or use of a new concomitant medication other than as described below is not allowed during the study unless required to treat an AE or is prescribed by a physician to treat another emergent medical issue.

1-DAY WASHOUT: Antacids; Sucralfate

1-Day Washout

H2 Receptor Antagonists (prescribed or over-the-counter [OTC]) (e.g. cimetidine, ranitidine, famotidine, and nizatidine).

14-Day Washout

Bile acid sequestrants (e.g., Welchol (colesevelam), cholestyramine, and colestipol)

Drugs with a known drug-drug interaction or a potential for a drug-drug interaction with colesevelam (cyclosporine, levothyroxine [and other thyroid replacement therapies], olmesartan medoxomil, phenytoin, warfarin)

Drugs with a narrow therapeutic index (e.g. warfarin, digoxin, theophylline)

Prokinetic agents (e.g. metoclopramide, tegaserod, erythromycin); anti-cholinergic and anti-muscarinic agents (e.g. dicyclomine, flavoxate, scopolamine, hyoscyamine, propantheline, oxybutynin, tolterodine, solifenacin, darifenacin, and trospium)

[inhaled ipratropium and tiotropium are permitted]

Antipsychotic agents (e.g., risperidone, haloperidol, droperidol, chlorpromazine, perphenazine, all phenothiazines, quetiapine, olanzapine, clozapine)

GABAergics (e.g., baclofen, valproic acid, gabapentin, pregabalin, benzodiazepine)

Calcium channel blockers (e.g., verapamil, nifedipine, diltiazem, amlodipine, felodipine, nicardipine, nimodipine, nisoldipine)

Beta blockers (e.g., metoprolol, timolol, atenolol, betaxolol)

All narcotics either alone or in combination (e.g., tramadol, codeine, morphine, propoxyphene, loperamide, diphenoxylate)

[narcotics used as anesthesia for an EGD require a 7 calendar day wash-out prior to the patient entering into the Pretreatment Period.]

Tricyclic antidepressants (e.g. amitriptyline, imipramine, and nortriptyline)

[Patients may take another single antidepressant (such as a selective serotonin reuptake inhibitor {SSRI}, or serotonin-norepinephrine reuptake inhibitor {SNRT} medication) as long as the dose has been stable for at least 30 days prior to the Screening Visit and the patient plans to continue a stable dose of the medications throughout the study. Use of more than 1 antidepressant medication is exclusionary.]

Other gastric-retentive drugs (e.g., Glumetza, Gralise)

Concomitant Medications:

Patients must have been on once-daily (QD) PPI therapy for at least 8 weeks before the Screening Visit.

Daily use of estrogens and/or low-dose aspirin (up to 162 mg/day) is permitted if, after an appropriate evaluation (e.g., history and physical exam), the Investigator believes these medications are not contributing to the patient's symptoms.

Nonsteroidal anti-inflammatory drugs (NSAIDs) are permitted for occasional use. Chronic use is not permitted.

Oral contraceptives containing ethinyl estradiol and norethindrone have a known drug-drug interaction with colesevelam. All female patients of childbearing potential using oral contraceptives with the ingredients listed above as birth control must agree to use another additional form of contraception from the date they sign the ICF until 24 hours after their final dose of study drug (e.g. condom).

TABLE 4
LOS ANGELES CLASSIFICATION OF ESOPHAGITIS

| Classification | Definition |
|---|---|
| Not | No breaks (erosions) in the esophageal mucosa. |
| Present | (However, edema, erythema, or friability may |
| | be present.) |
| Grade A | One or more mucosal breaks not more than 5 mm in |
| | maximum length. |
| Grade B | One or more mucosal breaks more than 5 mm in |
| | maximum length, but not continuous between the |
| | tops of 2 mucosal folds. |
| Grade C | Mucosal breaks that are continuous between the |
| | tops of 2 or more mucosal folds, but involve |
| | less than 75% of the esophageal circumference. |
| Grade D | Mucosal breaks that involve at least 75% of |
| | the esophageal circumference. |

Example of a “WHITE DIET”: Acceptable foods: Water, milk, chicken, fish, potatoes.

Weekly and End of Treatment Assessments

Degree of Relief Assessments

Administered weekly starting during Pretreatment Period: How would you rate your heartburn (a burning sensation in your chest, behind the breastbone) over the past 7 days? How would you rate your regurgitation (the feeling of stomach contents, either liquid or food, moving upwards to your throat or mouth) over the past 7 days?

Administered weekly during Treatment Period: Compared to before you started this study, how would you rate your overall GERD symptoms over the past 7 days?

Response Scale for all Degree of Relief Assessments: 1=Significantly relieved 2=Moderately relieved 3=Somewhat relieved 4=Unchanged 5=Somewhat worse 6=Moderately worse 7=Significantly worse

Global Treatment Satisfaction Assessment

Administered weekly during Treatment Period:

How would you rate your satisfaction with the study treatment? 1=Very dissatisfied 2=Dissatisfied 3=Neither satisfied nor dissatisfied 4=Satisfied 5=Very satisfied

Bothersomeness Assessments

Administered weekly starting during Pretreatment Period: How much were you bothered by heartburn (a burning sensation in your chest, behind the breastbone) over the past week? How much were you bothered by regurgitation (the feeling of stomach contents, either liquid or food, moving upwards to your throat or mouth) over the past week?

1=Not at all 2=A little bit 3=A moderate amount 4=A great deal 5=An extreme amount

Swallow Item Question

Administered End of Treatment Visit only:

How difficult were the tablets to swallow?

1=Not at all difficult 2=A little difficult 3=Moderately difficult 4=Extremely difficult

EQ-5D-3L

Health Questionnaire English version for the USA

By placing a checkmark in one box in each group below, please indicate which statements best describe your own health state today.

Mobility

I have no problems in walking about; I have some problems in walking about; I am confined to bed

Self-Care

I have no problems with self-care; I have some problems washing or dressing myself; I am unable to wash or dress myself ∇

Usual Activities (e.g. work, study, housework, family or leisure activities)

I have no problems with performing my usual activities; I have some problems with performing my usual activities; I am unable to perform my usual activities ∇

Pain/Discomfort

I have no pain or discomfort; I have moderate pain or discomfort; I have extreme pain or discomfort

Anxiety/Depression

I am not anxious or depressed; I am moderately anxious or depressed; I am extremely anxious or depressed □

To help people say how good or bad a health state is, we have drawn a scale (rather like a thermometer) on which the best state you can imagine is marked 100 and the worst state you can imagine is marked 0.

We would like you to indicate on this scale how good or bad your own health is today, in your opinion. Please do this by drawing a line from the box below to whichever point on the scale indicates how good or bad your health state is today.

QIDS-SR:

TABLE 5
SF-12v2 ® Health Survey Single-Item Acute Recall for Handheld Device

| | | | | | | |
|---|---|---|---|---|---|---|
| | Answer | Answer | Answer | Answer | Answer | Answer |
| Question Text | Text 1 | Text 2 | Text 3 | Text 4 | Text 5 | Text 6 |

| | | | | | |
|---|---|---|---|---|---|
| Your Health and Well- | | | | | |
| Being | | | | | |
| In general, would you | Excellent | Very good | Good | Fair | Poor |
| say your health is: | | | | | |
| The following | | | | | |
| questions are about | | | | | |
| activities you might | | | | | |
| do during a typical | | | | | |
| day. Does your | | | | | |
| health now limit you | | | | | |
| in these activities? | | | | | |
| If so, how much? | | | | | |
| Does your health | Yes, | Yes, limited | No, not | | |
| now limit you in | limited a | a little | limited at | | |
| moderate activities, | lot | | all | | |
| such as moving a | | | | | |
| table, pushing a | | | | | |
| vacuum cleaner, | | | | | |
| bowling, or playing | | | | | |
| golf? If so, how | | | | | |
| much? | | | | | |
| Does your health now | Yes, | Yes, limited | No, not | | |
| limit you in climbing | limited a | a little | limited at | | |
| several flights of | lot | | all | | |
| stairs? If so, | | | | | |
| how much? | | | | | |
| During the past week, | All of the | Most of the | Some of the | A little of | None of the |
| how much of the time | time | time | time | the time | time |
| have you had any of | | | | | |
| the following | | | | | |
| problems with your | | | | | |
| work or other regular | | | | | |
| daily activities as a | | | | | |
| result of your | | | | | |
| physical health? | | | | | |
| During the past week, | All of | Most of | Some of | A little of | None of |
| how much of the time | the time | the time | the time | the time | the time |
| were you limited in | | | | | |
| the kind of work or other | | | | | |
| activities as a result of | | | | | |
| your physical health? | | | | | |
| During the past week, | | | | | |
| how much of the time have | | | | | |
| you had any of the | | | | | |
| following problems with | | | | | |
| your work or other regular | | | | | |
| daily activities as a | | | | | |
| result of any emotional | | | | | |
| problems (such as feeling | | | | | |
| depressed or anxious)? | | | | | |
| During the past week, | All of | Most of | Some of | A little of | None of |
| how much of the time have | the time | the time | the time | the time | the time |
| you accomplished less than | | | | | |
| you would like as a result | | | | | |
| of any emotional problems | | | | | |
| (such as feeling depressed | | | | | |
| or anxious)? | | | | | |
| During the past week, | All of | Most of | Some of | A little of | None of |
| how much of the time have | the time | the time | the time | the time | the time |
| you done work or other | | | | | |
| activities less carefully | | | | | |
| than usual as a result of | | | | | |
| any emotional problems (such | | | | | |
| as feeling depressed | | | | | |
| or anxious)? | | | | | |
| During the past week, | Not at | A little bit | Moderately | Quite a bit | Extremely |
| how much did pain interfere | all | | | | |
| with your normal work | | | | | |
| (including both work outside | | | | | |
| the home and housework)? | | | | | |
| These questions are about | | | | | |
| how you feel and how things | | | | | |
| have been with you during | | | | | |
| the past week. For each | | | | | |
| question. please give the | | | | | |
| one answer that comes closest | | | | | |
| to the way you have been feeling. | | | | | |
| How much of the time | All of | Most of | Some of | A little | None of |
| during the past | the time | the time | the time | of the time | the time |
| week have you felt calm | | | | | |
| and peaceful? | | | | | |
| How much of the | All of | Most of | Some of | A little of | None of |
| time during the past | the time | the time | the time | the time | the time |
| week did you have | | | | | |
| a lot of energy? | | | | | |
| How much of the | All of | Most of | Some of | A little of | None of |
| time during the past | the time | the time | the time | the time | the time |
| week have you felt | | | | | |
| downhearted and | | | | | |
| depressed? | | | | | |
| During the past week, | All of | Most of | Some of | A little of | None of |
| how much of the time | the time | the time | the time | the time | the time |
| has your physical | | | | | |
| health or emotional | | | | | |
| problems interfered | | | | | |
| with your social | | | | | |
| activities (like visiting | | | | | |
| with friends, | | | | | |
| relatives, etc.)? | | | | | |

mRESQ-eD (Modified Reflux Symptom Questionnaire Electronic Diary)

The primary objective of the cognitive debriefing interviews is to evaluate the content validity of the mRESQ-eD and qualitatively examine how rGERD patients think about and define a meaningful change in symptom improvement.

A subset of participants (30) participate in cognitive debriefing interviews. They are divided into 3 cohorts.

Cohort 1: Patients in Cohort 1 are to complete the mRESQ-eD daily for the duration of the Pretreatment Period and at least four weeks of the Treatment Period (n=20). Patients in Cohort 1 must have completed the mRESQ-eD rather than the RESQ to be eligible to participate in the cognitive interviews.

Cohort 2: Patients in Cohort 2 have erosive esophagitis (evaluated by EGD) and/or evidence of pathological acid reflux (evaluated with the Bravo® device), and be mRESQ-eD naïve (n=5). Screen failures are not eligible for Cohort 2.

Cohort 3: Patients in Cohort 3 do not have erosive esophagitis (evaluated by EGD) and/or evidence of pathological acid reflux (evaluated with the Bravo® device), and be mRESQ-eD naïve (n=5).

Cohorts are scheduled for interviews during the clinical trial visits outlined below.

Cohort 1 Schedule Interview to Occur During: Week 4 or Week 8 Visit

Cohort 2 Schedule Interview to Occur During: Pretreatment Visit

Cohort 3 Schedule Interview to Occur During: Any time after screen for EE failed. Interviews are scheduled to coincide with other Cohort 1 or 2 interviews as scheduled for the same day.

Regardless of cohort or interview timing, all interview participants are cognitively debriefed on the mRESQ-eD and asked to provide input on what constitutes a meaningful change in symptom improvement on the mRESQ-eD.

Interview Materials

TABLE 6
PPI DOSE LEVELS DURING STUDY PARTICIPATION
Acceptable Dose Levels of the Various PPIs for Participation in the Trial¹

| | | Acceptable | | Acceptable |
|---|---|---|---|---|
| | | Dose Levels for | | Dose Level |
| PPI | Common | Non-Erosive | | for Erosive |
| Generic Name | Brand Name(s) | Esophagitis² | | Esophagitis |
| dexlansoprazole | Dexilant ®, | 30-60 | mg QD | 60 mg QD |
| | Kapidex ® | | | |
| esomeprazole | Nexium ® | 20-40 | mg QD | 40 mg QD |
| lansoprazole | Prevacid ® | 15-30 | mg QD | 30 mg QD |
| omeprazole | Prilosec ® | 20 | mg QD | 20 mg QD |
| pantoprazole | Protonix ® | No indication | | 40 mg QD |
| rabeprazole | AcipHex ® | 20 | mg QD | 20 mg QD |

¹The dose levels are acceptable provided that the PPI treatment has been optimized. (Optimized means treatment that, according to the Investigator's judgment, could not be further improved by changing the brand or timing of PPIadministration.)
²For dexlansoprazole, esomeprazole, and lansoprazole, the approved dose level for non-erosive esophagitis is the first one that is listed. However, either dose is acceptable for enrollment in the study.

Outcomes

This is a Randomized, placebo-controlled, double-blind, dose-ranging study; 70 patients/arm study.

Weekly Heartburn Severity Score (WHSS)

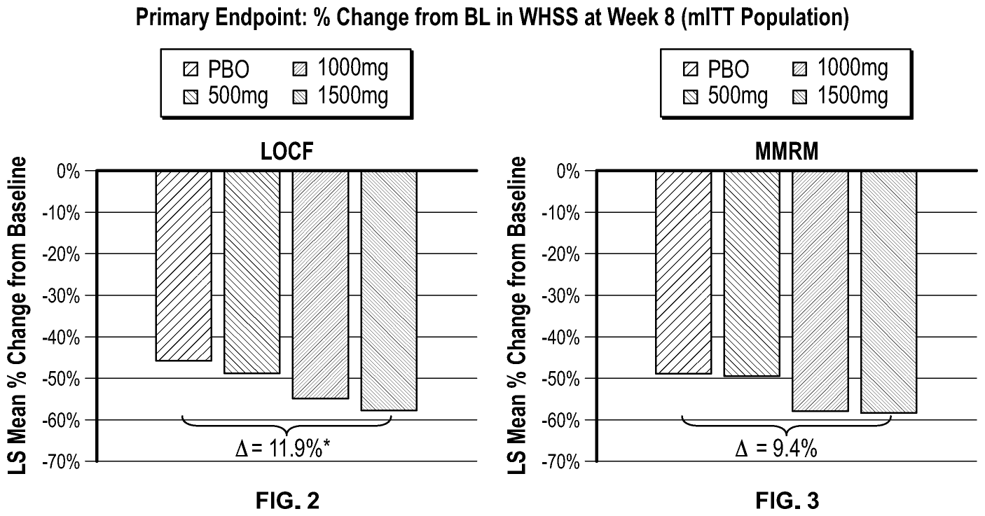

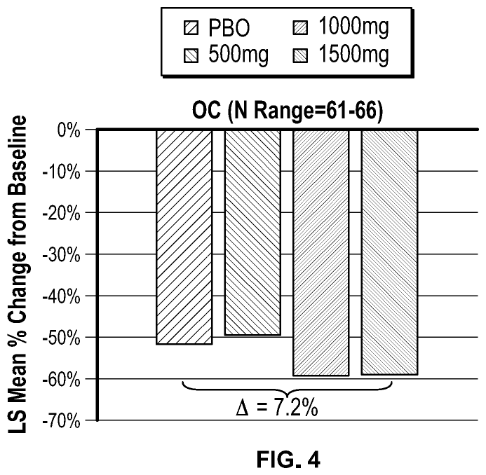

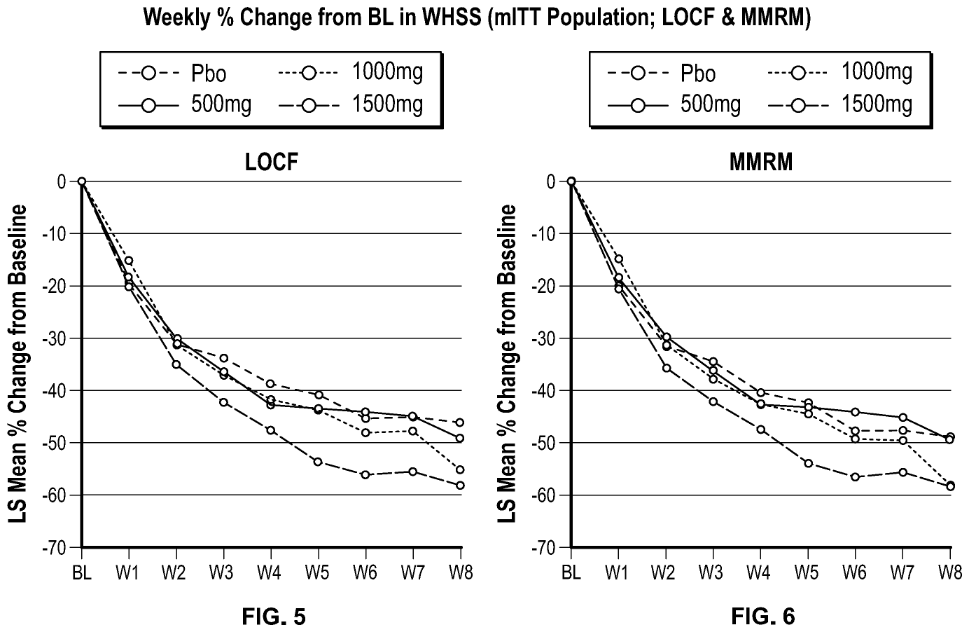

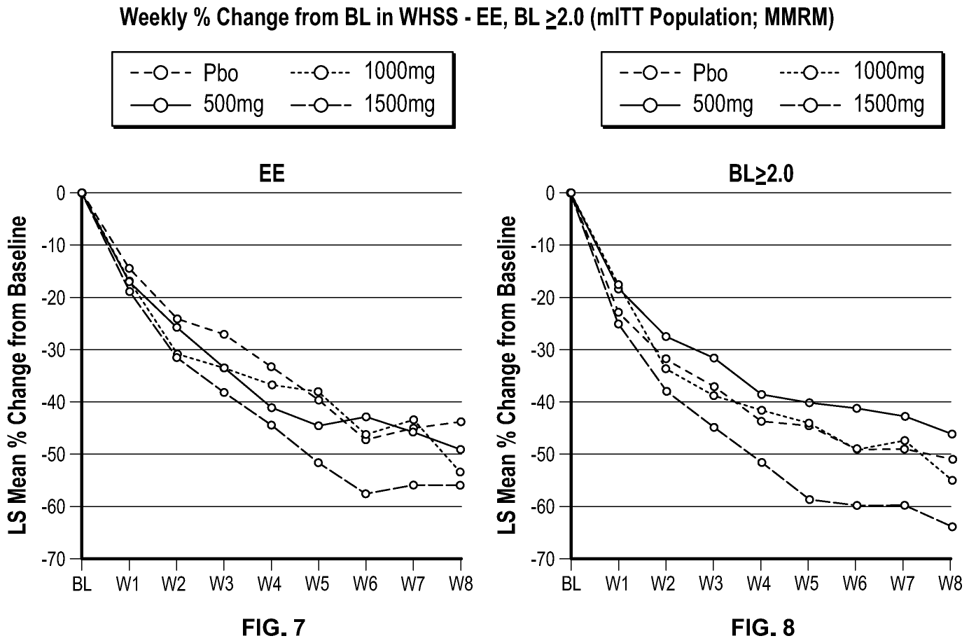

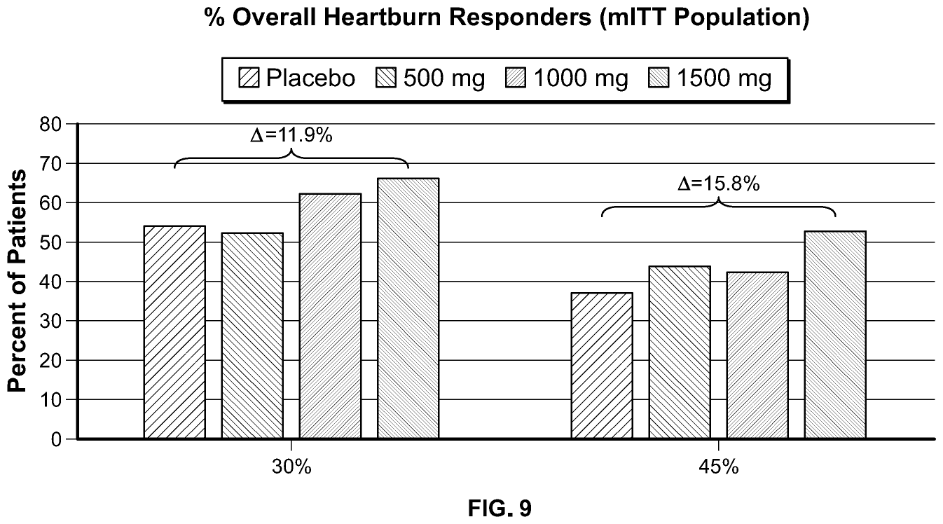

Weekly Heartburn Severity Score (WHSS) is defined as the weekly average of the Daily Heartburn Severity Score (DHSS). DHSS is defined as the maximum of the 3 items measuring heartburn from a particular day, as collected with the mRESQ-eD. Data were collected for the modified-intent-to-treat (mITT) group and the patient populations are presented as LOCF (last observation carried forward); MMRM (mixed model repeated measures); EE (patients with erosive esophagitis), and OC (observed cases). FIG. 2-FIG. 8 show that patients taking 1500 mg of IW-3718 twice daily had a clinically significant percent change from baseline (BL) at week 8 (between 7.2% and 12.7%) in all 3 patient populations. There was also a clinically significant dose dependent response to IW-3718 in all 3 populations. These conclusions are true for EE patients as well as for baseline above 0 or above or equal to 2.5. P-values are based on a pairwise comparison versus placebo in an ANCOVA model with fixed effect terms for treatment group and esophagitis and baseline value as covariate. Trend test nominal P=0.0225, performed using linear contrast statement. EE are patients with erosive esophagitis.

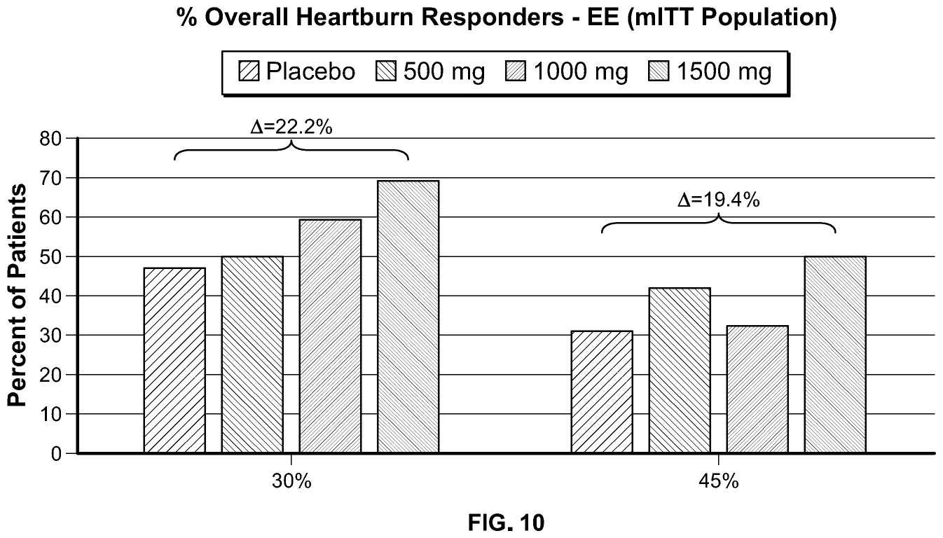

Data for percent overall heartburn responders for the mITT population are shown in FIG. 9-FIG. 10. An Overall Heartburn Responder had a decrease of at least 30% (or 45%) in WHSS for at least 4 of the 8 treatment weeks, including at least 1 of the last 2 weeks. Again, a clinically significant percent of patients taking 1500 mg of IW-3718 twice daily was overall heartburn responders, as compared to those on placebo (between 11.9% to 15.8% more than those on placebo; for EE patients between 19.4% to 22.2% more than those on placebo). There was also a clinically significant dose dependent response to IW-3718.

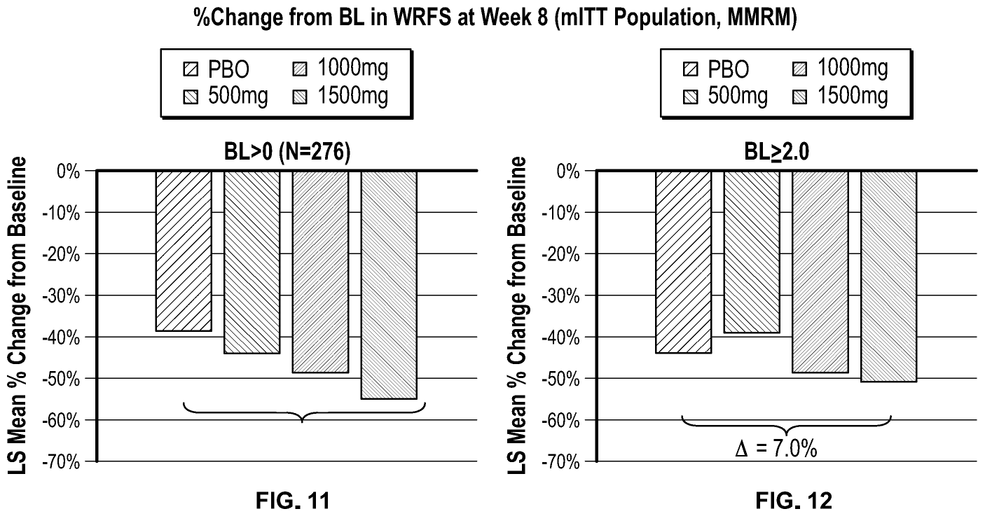

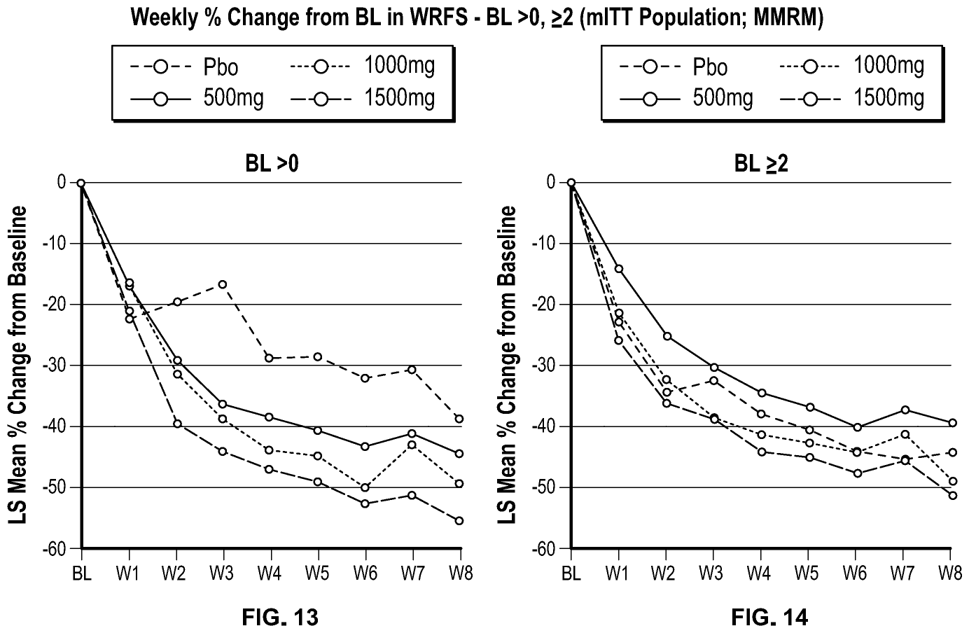

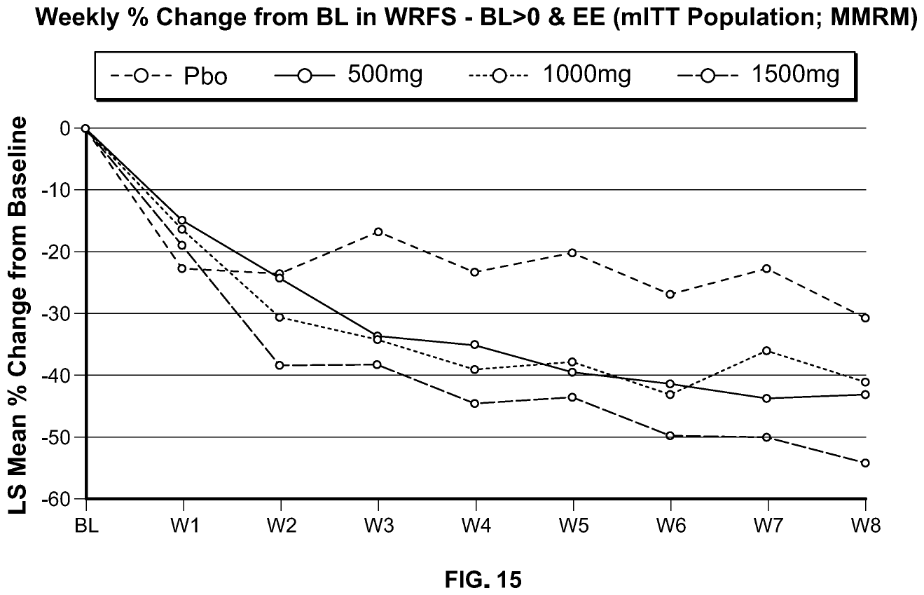

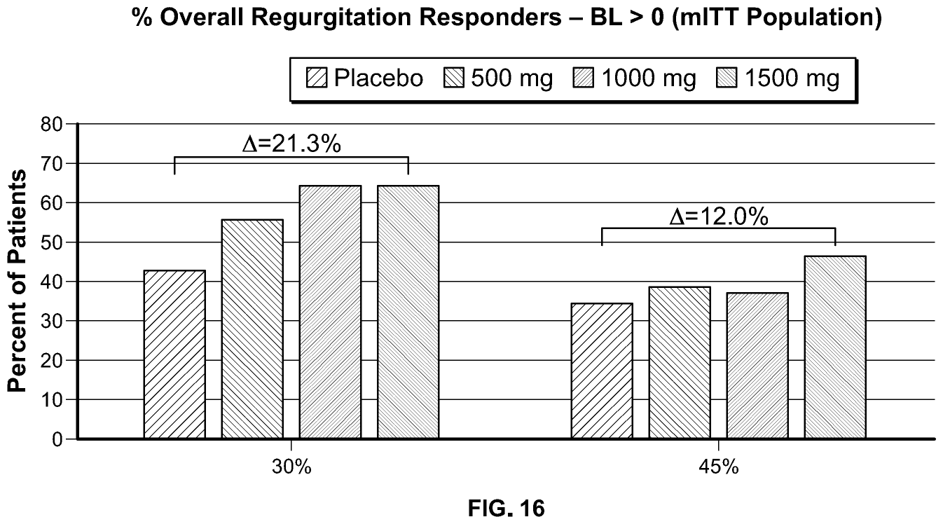

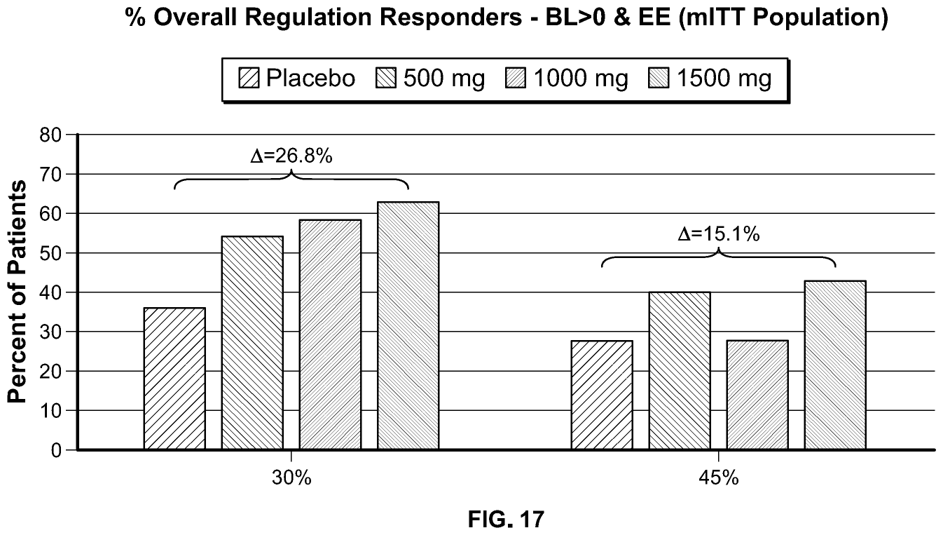

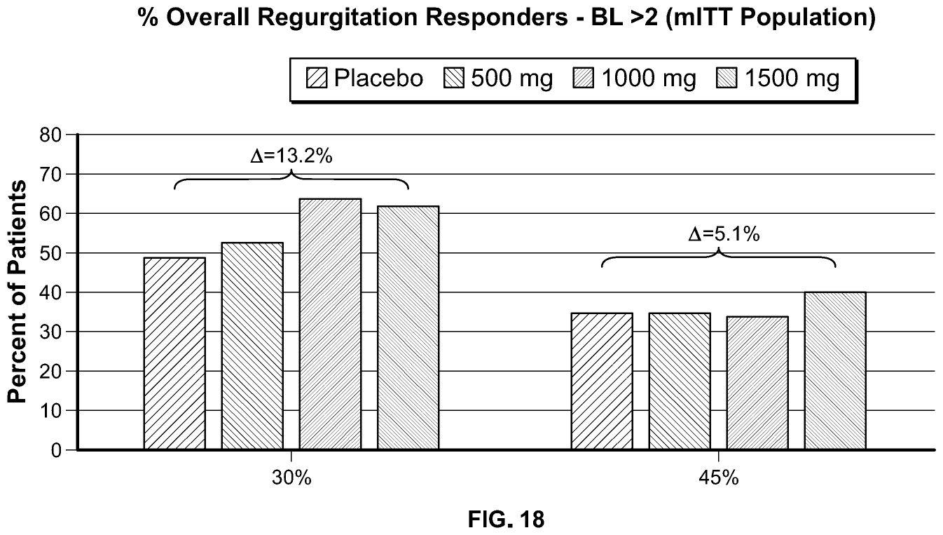

Data for percent change from baseline in weekly regurgitation frequency score (WRFS) for mITT patients (MMRM, or mixed model repeated measures) are shown in FIG. 11-FIG. 15. A clinically significant decrease from baseline was observed in patients taking IW-3718, in a dose-dependent fashion. This is true for EE patients, for BL greater than 0, and BL greater than or equal to 2. Weekly Regurgitation Frequency Score (WRFS) is defined as the weekly average of the Daily Regurgitation Frequency Score (DRFS). DRFS is defined as the maximum of the 2 items measuring regurgitation from a particular day, as collected with the mRESQ-eDData for percent overall regurgitation responders for the mITT population are shown in FIG. 16-FIG. 18. A clinically significant percent increase in overall regurgitation responders (an Overall Regurgitation Responder had a decrease of at least 30% (45%) in WRFS for 4 of the 8 treatment weeks, including 1 of the last 2 weeks) was observed in patients taking IW-3718 (between 12.0% to 26.8%), in a dose-dependent fashion. Again, this is true for EE patients, for BL greater than 0, and BL greater than or equal to 2.

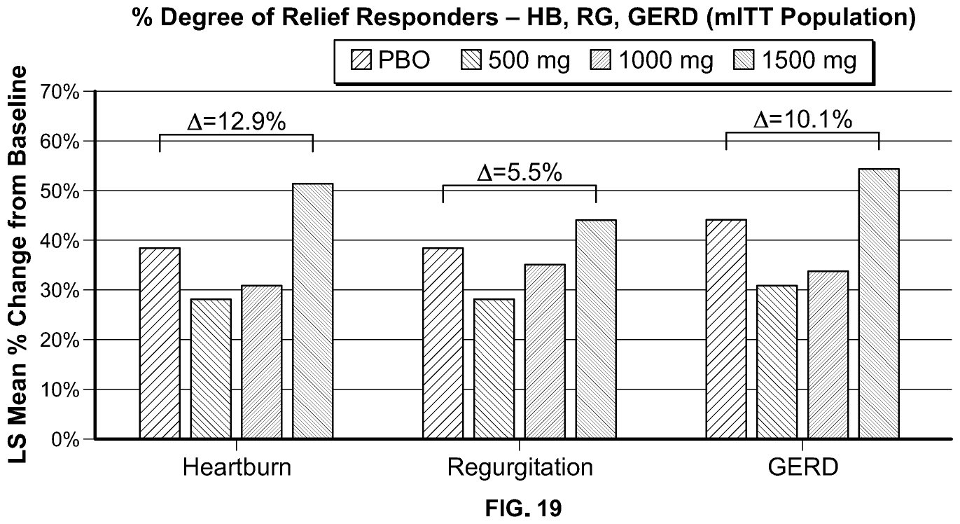

Data for percent degree of relief responders for heartburn (HB), regurgitation (RG) and GERD for the mITT population are shown in FIG. 19. A % Degree of Relief Responder was “significantly” or “moderately” relieved for 4 of the 8 treatment weeks. Clinically significant response, in a dose dependent manner, were observed for patients taking IW-3718, with an increase between 5.5% to 12.9% over placebo.

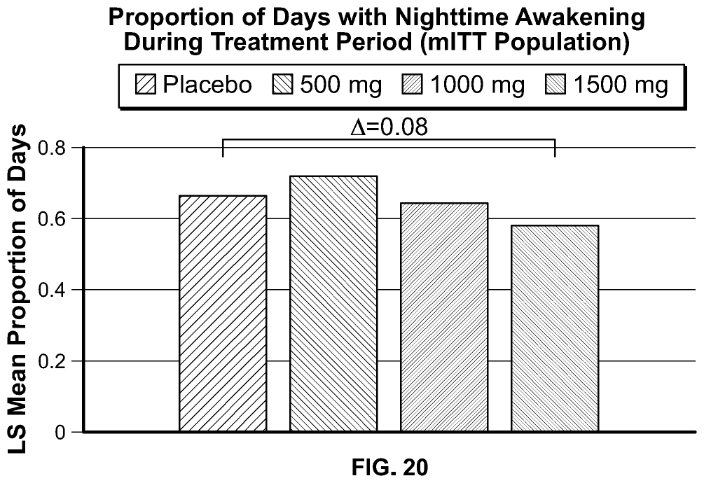

Improvement in nighttime awaking during treatment period was also observed for the mITT population. FIG. 20.

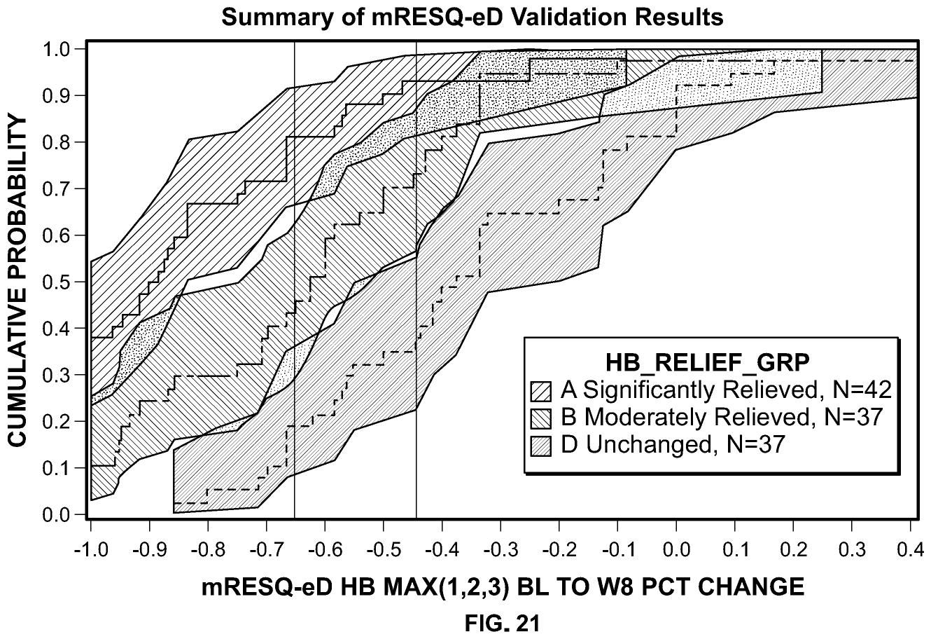

FIG. 21 shows a summary of mRESQ-eD validation results. A large number of participants on IW-3718 obtained relief of their GERD symptoms.

Overall, there was no safety issues. There were no deaths, very few serious AE (1-2 per arm and all unrelate), few dropouts due to AE (1-3 per arm; 2 IW3718 patients withdrew due to constipation. TEAEs rate for patients on IW-3718 was 42-52% and for placebo was 41%. Constipation rate for patients on IW-3718 was 7.4-8.5%, similar to patients on placebo (7.1%).

### Example 2: A Single Center, Open-Label, Randomized, Single Dose, 3-Way Scintigraphy Study in Healthy Subjects with 3 Periods, Each with a Different Breakfast Composition Designed to Evaluate the In Vivo Performance of IW-3718 Compared with Immediate Release Colesevelam in the Fed State

The purpose of this study is to compare the gastro-retentive performance of a 500 mg IW-3718 tablet versus the immediate release (IR comparator product Cholestagel [colesevelam; 625 mg]) in the fed state. The gastro-retentive performance of both drugs is investigated following administration after breakfasts with different fat and calorie content.

The recommended dose for Cholestagel is 6×625 mg tablets per day; the dose selected for this study is 1×625 mg tablet in each of the 3 periods. This dose is well within the recommended daily dose, thereby limiting the exposure to healthy volunteers yet sufficient to observe tablet disintegration via scintigraphic analysis.

The purpose of this study is to show a difference between the gastric retention of IR colesevelam and IW-3718 after breakfasts differing in fat and calorie content. Therefore it is an advantage to minimize variability; and as such, healthy volunteers are the most appropriate population for this study.

The European Medicines Agency (EMA) suggest including subjects in the age group 18 to 55 years with normal weight who are non-smokers without a history of alcohol or drug abuse. The latter criteria are proposed to avoid interaction on drug metabolism and to avoid non-compliance.

As GERD affects equally in men and women and presents around 20 to 50 years of age, male and female subjects are eligible for this study. Pregnancy tests are conducted at Screening and Admission (Day −1) and a positive result excludes the subject from the study.

In an interaction study in healthy volunteers, Cholestagel reduced the Cₘₐₓ of norethindrone as well as the AUC and Cₘₐₓ of ethinylestradiol when administered simultaneously with the oral contraceptive pill. This interaction was also observed when Cholestagel was administered one hour after the oral contraceptive pill. Therefore women who are on hormonal methods of contraception (oral, injectable, transdermal, intravaginal, intrauterine hormone-release system) are not permitted in this study.

IW-3718 (colesevelam) is an orally administered, non-absorbed, non-digestible polymer that binds bile acids in the GI tract. Colesevelam was approved in 2000 in the US as the active ingredient in Welchol, a drug indicated as an adjunct to diet and exercise for reduction of elevated LDL-c in adults with primary hyperlipidaemia. Colesevelam is currently available as an IR formulation only.

In clinical trials of colesevelam (Welchol), the most common adverse reactions included constipation, dyspepsia, and nausea. Other postmarketing adverse reports include bowel obstruction, dysphagia, esophageal obstruction, faucal impaction, hypertriglyceridemia, pancreatitis, and increased transaminases.

The dosage forms contain a radionuclide (not more than 1 megabecquerel (MBq; 27 μCi) ¹¹¹In and not more than 4 MBq (108 μCi)^{99m}Tc so subjects are exposed to ionizing radiation. In addition, to capture gamma scintigraphic images, two anatomical markers are taped to the skin on each dosing occasion. Each of these contain ^{99m}Tc (not more than 0.05 MBq [1.35 μCi]), resulting in an effective dose of 0.04 millisievert (mSv). The effective dose that each subject receives from one administration is not to exceed 0.53 mSv. This is approximately 3 months of the average natural source of background radiation dose received in the UK each year (2.2 mSv; data obtained from the Public Health England [PHE] Website). This equates to slightly less than the radiation dose that would result from an x-ray of the abdomen (0.7mSv).

Electrocardiogram stickers on the subjects' chests and limbs may cause some local irritation and may be uncomfortable to remove but subjects are closely monitored to ensure any local irritation does not persist.

The primary objective of the study is: To evaluate the in vivo gastro-retention properties of IW-3718 compared with IR colesevelam using scintigraphic methods.

The secondary objectives of the study are: To determine the gastro-retention properties of IW-3718 following 3 different breakfasts

To determine the disintegration profile of IW-3718 following 3 different breakfasts

To evaluate the disintegration profile of IR colesevelam tablet following 3 different breakfasts

To provide additional information on the safety and tolerability of IW-3718 after oral administration

Endpoints

Primary Endpoints: A comparison of IW-3718 with IR colesevelam on complete gastric emptying time T₉₀ after subjects consume a low-fat high calorie breakfast.

Secondary Endpoints:

A comparison of the in vivo transit and disintegration of IW-3718 following different breakfasts by measuring the following scintigraphic parameters: gastric emptying, time and location of initial and complete disintegration, rate of erosion.

A comparison of the in vivo transit and disintegration of IR colesevelam tablet following different breakfasts by measuring the following scintigraphic parameters: gastric emptying, time and location of initial and complete disintegration.

To collect further information about the safety and tolerability of IW-3718 by assessing: physical examination, safety laboratory tests, vital signs, ECGs, and AEs.

Study Design

Study Plan

This is a single center, open-label, randomized, single dose 3-way scintigraphy study in healthy subjects with 3 periods, each with a different meal composition. It is planned to enroll 18 healthy subjects to collect data on 15 evaluable subjects at the end of the clinical study. A subject is considered evaluable if they have at least one scintigraphic evaluation.

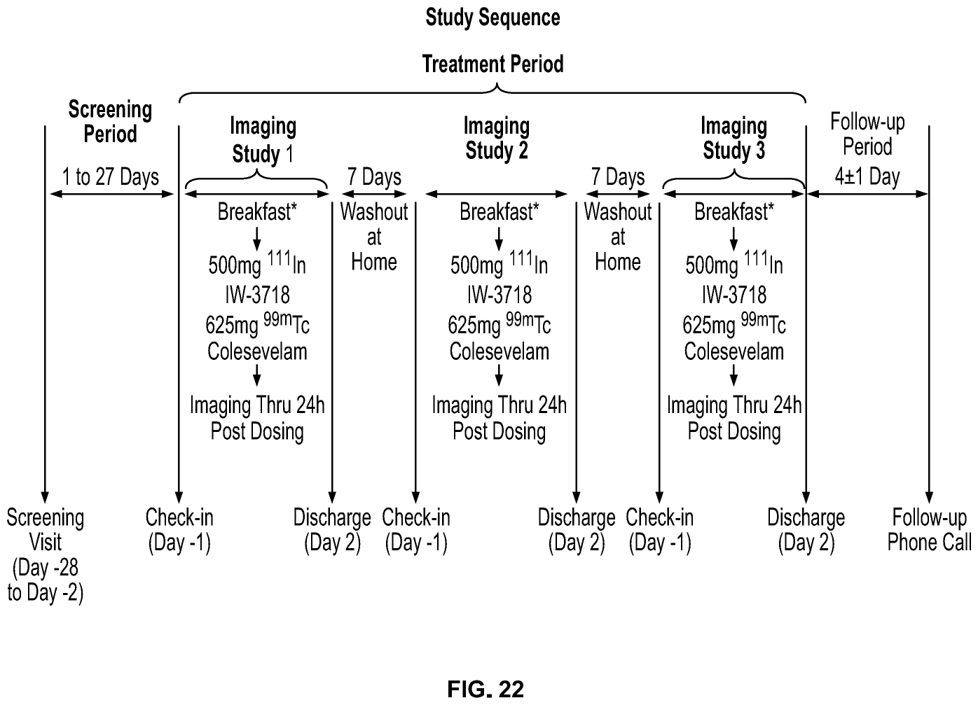

Subjects are to undergo screening procedures to determine their eligibility for the study at the Screening Visit (which can occur from Day −28 to Day −2). Each period follows the same study design. FIG. 22. Eligible subjects are admitted to the imaging facility for an overnight stay on 3 occasions, each at least 7 days, but no more than 3 weeks, apart. Subjects are admitted in the evening on the day before dosing (Day −1) and remain onsite until 24 h post-dose. On each of the study days the subjects receive 1 of 3 different breakfasts:

Regimen A: high-fat, high calorie—1000 calories with 50% fat

Regimen B: low-fat, high calorie—1000 calories with 33% fat

Regimen C: low-fat, low-medium calorie—500 calories with 33% fat

Subjects are randomly assigned in a 1:1:1:1:1:1 ratio to 1 of the 6 possible meal sequences. Table 7. In each period, subjects receive one tablet of 500 mg ¹¹¹In-labelled IW-3718 and one tablet of 625 mg IR ⁹⁹mTc-labelled colesevelam approximately 30 min after completion of the appropriate breakfast.

Following an overnight fast of 10 hours (h), subjects start the assigned breakfast 30 minutes (min) before administration of the IMPs, and should complete the meal evenly over a 25 min period; subjects are expected to consume 100% of the meal. The IMPs are administered 30 min after the start of breakfast. The test products are administered with 240 mL water and no further food is permitted until 4 h post-dose. Water can be taken as desired except for 1 h before and after IMP administration. Subjects receive standardized meals at the same time in each period of the study.

On each of the 3 study periods each subject receives one of the following regimens in an order according to the randomization schedule:

TABLE 7
Investigational Medicinal Product (IMP)

| Regimen | IMP Dose | Food Status |
|---|---|---|
| A | ¹¹¹In-IW-3718 Tablet | 30 minutes after |
| | (500 mg labelled with | start of high-fat, |
| | NMT 1 MBq ¹¹¹In) | high calorie |
| | ^{99m}Tc-colesevelam Tablet | breakfast |
| | (625 mg labelled with | |
| | NMT 4 MBq ^{99m}Tc) | |
| B | ¹¹¹In-IW-3718 Tablet | 30 minutes after |
| | (500 mg labelled with | start of low-fat, |
| | NMT 1 MBq ¹¹¹In) | high calorie |
| | ^{99m}Tc-colesevelam Tablet | breakfast |
| | (625 mg labelled with | |
| | NMT 4 MBq ^{99m}Tc) | |
| C | ¹¹¹In-IW-3718 Tablet | 30 minutes after |
| | (500 mg labelled with | start of low-fat, |
| | NMT 1 MBq ¹¹¹In) | low-medium calorie |
| | ^{99m}Tc-colesevelam Tablet | breakfast |
| | (625 mg labelled with | |
| | NMT 4 MBq ^{99m}Tc) | |

Anterior and posterior scintigraphic images are acquired immediately after administration of IMPs, and then at selected time points until 24 h post-dose.

Using a computer-generated randomization schedule, subject numbers are allocated to sequence. Table 8. The allocation is balanced with 3 subjects receiving each sequence.

TABLE 8
Treatment Sequences

| Treatment | Treatment | Treatment | Treatment |
|---|---|---|---|
| Sequence | Period 1 | Period 2 | Period 3 |
| 1 | Regimen A | Regimen B | Regimen C |
| 2 | Regimen A | Regimen C | Regimen B |
| 3 | Regimen B | Regimen A | Regimen C |
| 4 | Regimen B | Regimen C | Regimen A |
| 5 | Regimen C | Regimen B | Regimen A |
| 6 | Regimen C | Regimen A | Regimen B |

Subject Withdrawal

Subjects are withdrawn for the following reasons:

Experiencing a serious or severe AE including but not limited to: corrected QT (QTc) interval of >500 ms or increase in QTc interval of >60 ms from baseline (confirmed following a repeat ECG); alanine aminotransferase (ALT) concentration>3× the upper limit of the reference range; Termination of the study; Upon the subject's request (withdrawal of consent); Significant deviation from the protocol; Concurrent illness or requirement for prohibited medication; At the discretion of the investigator.

Any subject discontinuing the study prematurely because of an IMP-related AE or termination of the study is considered to have completed the study, and is not replaced. Subjects withdrawing for other reasons may be replaced. Up to 3 replacement subjects may be enrolled into the study. The maximum number of subjects that may be dosed is 21. Additional subjects enrolled are dosed with the next planned regimen of the withdrawn subject, and they do not receive any regimen that the withdrawn subject has already received. A subject is considered to be an evaluable subject if they have at least 1 scintigraphic evaluation.

Selection of Subjects

A full medical history is obtained from each subject's general practitioner (GP) within the last 12 months, prior to enrolment in the study. Each subject's GP is informed of their participation in the study.

Before subjects are admitted to the clinic, The Over Volunteering Prevention System (TOPS) is checked to ensure that each subject has not participated in a study at another site within at least 3 months of the dosing date.

Subjects are provided with a written explanation of the study at least the day before the Screening Visit. A physician or nurse explains to each subject the nature of the study, its purpose, expected duration and the benefits and risks involved in study participation. Subjects are informed that, for safety reasons, brief details of their involvement in the study may be revealed to other units and companies that carry out clinical studies in the local area. Subjects are then given the opportunity to ask questions and are informed of their right to withdraw from the study without prejudice. After this explanation and before entering the study, the subject voluntarily signs an informed consent form (ICF). The advice of the National Radiological Protection Board (1993) concerning the exposure to ionizing radiation of women who may be pregnant, is discussed with female subjects. They are tested using a sensitive urine pregnancy screening kit prior to each study period. Any subject found to be pregnant, or in whom pregnancy cannot reasonably be excluded, is to be withdrawn from the study.

Inclusion Criteria

Healthy males or non-pregnant, non-lactating healthy females

Age 18 to 55 years of age

Body mass index of >18.5 to ≤32.0 kg/m² or, if outside the range, considered not clinically significant by the investigator

Subjects must demonstrate their ability to swallow an empty size 000 capsule

Must be willing and able to communicate and participate in the whole study

Must provide written informed consent

Subject agrees to refrain from making any major lifestyle changes (e.g., starting a new diet or changing their exercise pattern) from time of signature of the ICF until after the follow-up call

Must agree to use an adequate method of contraception.

Females using hormonal method of contraception (oral, injectable, transdermal, intravaginal, intrauterine hormone-release system) are not permitted in this study.

Exclusion Criteria

Subjects who have received any IMP in a clinical research study within the previous 3 months

Subjects who are study site employees, or immediate family members of a study site or sponsor employee

Subjects who have previously been enrolled in this study

History of any drug or alcohol abuse in the past 2 years

Regular alcohol consumption in males>21 units per week and females>14 units per week (1 unit=½ pint beer, 25 mL of 40% spirit or a 125 mL glass of wine)

Current smokers and those who have smoked within the last 12 months. A breath carbon monoxide reading of greater than 10 ppm at Screening.

Current users of e-cigarettes and nicotine replacement products and those who have used these products within the last 12 months

Females of childbearing potential who are pregnant or lactating (female subjects must have a negative serum pregnancy test at Screening and a negative urine pregnancy test at Admission). A woman is considered of childbearing potential unless she is permanently sterile (hysterectomy, bilateral salpingectomy or bilateral oophorectomy) or is postmenopausal (had no menses for 12 months without an alternative medical cause and a serum follicle-stimulating hormone [FSH] concentration>40 IU/L)

Radiation exposure, including that from the present study, excluding background radiation but including diagnostic x-rays and other medical exposures, exceeding 5 mSv in the last 12 months or 10 mSv in the last 5 years. No occupationally exposed worker, as defined in the Ionizing Radiation Regulations 1999, shall participate in the study

Clinically significant abnormal biochemistry, hematology, coagulation or urinalysis test results at Screening as judged by the investigator

Subject has elevated (>1.25× upper limit of normal) levels of ALT, aspartate aminotransferase or creatinine at Screening

Positive drugs of abuse test result at Screening

Positive hepatitis B surface antigen (HBsAg), hepatitis C virus antibody (HCV Ab) or human immunodeficiency virus (HIV) results at Screening History of clinically significant cardiovascular, renal, hepatic, respiratory, metabolic, hematological, pulmonary, musculoskeletal, dermatological, urogenital, eye, ear, nose and throat, psychiatric, neurological or particularly GI disease, especially peptic ulceration, GI bleeding, ulcerative colitis, Crohn's Disease, bowel obstruction or Irritable Bowel Syndrome

History of gall bladder stones, including asymptomatic gall bladder stones, biliary colic, biliary tract obstruction

Subject has history of malignancy, diagnosed or known to be active or actively treated within the past 5 years, other than resected lesions of low malignant potential, such as basal cell skin cancers

Subject has a 12-lead ECG demonstrating severe bradycardia (heart rate<40 bpm) or average QT interval corrected for heart rate using Fridericia's formula (QTcF)

≥450 ms for male subjects or ≥470 ms for female subjects (if on the initial ECG, QTcF exceed criteria, then the ECG is repeated 2 more times, and the average of the 3 QTcF values are used to determine the subject's eligibility)

Serious adverse reaction or serious hypersensitivity to any drug or the formulation excipients

Presence or history of clinically significant allergy requiring treatment, as judged by the investigator. Hay fever is allowed unless it is active

Donation or loss of greater than 400 mL of blood within the previous 3 months

Subjects who are taking, or have taken, any prescribed medications (other than topical medications) in the 14 days before IMP administration. Exceptions may apply on a case by case basis, if considered not to interfere with the objectives of the study.

Subjects who are taking, or have taken, any over-the-counter drug (other than 4 g per day paracetamol and topical medications) or herbal remedies in the 7 days before IMP administration. Exceptions may apply on a case by case basis, if considered not to interfere with the objectives of the study.

Subject has undergone a surgical procedure during the 30 days before Day −1, other than minor dermatological procedures

History of GI surgery including cholecystectomy (with the exception of appendectomy unless it was performed within the previous 12 months)

Subject has an acute or chronic condition that, in the investigator's opinion, would limit the subject's ability to complete or participate in this clinical study

Acute diarrhea or constipation in the 7 days before the predicted first study day. If Screening occurs >7 days before the first study day, this criterion is to be determined on first study day. Diarrhea is defined as the passage of liquid faces and/or a stool frequency of greater than 3 times per day. Constipation is defined as a failure to open the bowels more frequently than every other day

History of dysphagia, swallowing disorders or GI obstruction

Failure to satisfy the investigator of fitness to participate for any other reason

Healthy subjects who do not meet the inclusion/exclusion criteria for a study should not be enrolled into the study without exception.

Contraception

Male subjects who are sexually active with a partner of child bearing potential must use, with their partner, a condom plus an approved method of highly effective contraception from the time of informed consent until 90 days after study discharge as the IMP is radiolabeled. Female subjects who are sexually active and of childbearing potential must use, with their partner, an approved method of highly effective contraception from the time of informed consent until 30 days after study discharge as the IMP is radiolabeled.

The following methods are acceptable: Implantable intrauterine device (IUD) and Surgical sterilization (for example, vasectomy or bilateral tubal ligation). Alternatively, true abstinence is acceptable when it is in line with the subject's preferred and usual lifestyle. Periodic abstinence e.g., calendar, ovulation, symptothermal, post-ovulation methods and withdrawal are not acceptable methods of contraception. If a subject is usually not sexually active but becomes active, they, with their partner, must comply with the contraceptive requirements detailed above.

Female subjects who are not of childbearing potential do not need to use any methods of contraception. A woman is considered of childbearing potential unless post-menopausal or permanently sterile. Permanent sterilization methods include hysterectomy, bilateral salpingectomy and bilateral oophorectomy. A post-menopausal state is defined as no menses for 12 months without an alternative medical cause and confirmed by a FSH result of ≥40 IU/L.

Exposure to Partners During the Study

There is a significant risk of radiation exposure through the ejaculate (which also applies to vasectomized males) that might be harmful to the sexual partners (both male and female), including pregnant partners of male subjects. Therefore, a condom should be used by all male subjects throughout the study and for 90 days after study discharge.

Male subjects should not donate sperm for the duration of the study and for at least 90 days after the last dose.

Subjects are instructed that if they/their partner become pregnant during the study this should be reported to the investigator. The investigator should also be notified of pregnancy occurring during the study but confirmed after completion of the study. In the event that a subject/subject's partner is subsequently found to be pregnant after the subject is included in the study, then consent is to be sought from the partner and, if granted, any pregnancy is to be followed and the status of mother and/or child is to be reported. Any subject reporting a pregnancy during the study are discontinued from the study treatment.

Additional Study Restrictions

Subjects must not eat anything likely to disturb GI transit (e.g., spicy or high-fat meals such as curry or fish and chips or foods of a high-fiber content such as All-Bran) for 24 h prior to Admission until 24 h after the final dose.

Subjects must abstain from alcohol during the 48 h prior to Screening and the 48 h prior to Admission until 24 h after the final dose.

Subjects must not smoke or use nicotine-containing products from 12 months prior to admission until 24 h after the final dose.

Subjects must not drink liquids or eat food containing caffeine from 48 h prior to Admission until 24 h after the final dose.

Subjects must not drink liquids or eat food containing grapefruit or cranberry from 72 h prior to Admission until 24 h after the final dose.

Subjects should refrain from eating food containing poppy seeds for 48 h prior to Screening and for 48 h prior to Admission until 24 h after the final dose.

Subjects must not take part in any unaccustomed strenuous exercise from the 72 h before the Screening Visit and then from 72 h prior to Admission until discharge from the study.

Study Procedures

Screening

Within the 28 days preceding first dose, all subjects are required to undergo a Screening Visit. If the start of the study is delayed for any reason so that the interval between Screening and first dose exceeds 28 days, all or part of the screening procedures may be repeated at the discretion of the investigator. Subjects previously screened for another study or generically screened may participate in this study provided they meet the subject selection criteria. Screening data from the previous study or visit is considered sufficient to satisfy the requirements of this study. Procedures required by this protocol is only done if they were not performed for the previous study or visit, and may be done on Admission Day −1. All screening data must be obtained within 28 days prior to administration of study medication, as stipulated above.

Subject Re-Screening

This study permits the re-screening of a subject who has discontinued the study as a pre-treatment failure (i.e., subject has not been randomized/has not been treated); the reason for failure must be temporary and expected to resolve. If re-screened, the subject must be re-consented.

Admission and Pre-dose Procedures

The identity of the subjects is confirmed at Admission on Day −1 and pre-dose on Day 1. In addition, the ongoing eligibility of subjects is re-assessed at Admission on Day −1 and pre-dose on Day 1. Reserve subjects for the first dose occasion, in any group, are not required admission procedures to be repeated, if dosing is within 2 days.

The subjects are admitted to the clinical unit on the evening of the day before dosing (Day −1).

Study Day Procedures

The total blood volume for each subject is not to exceed 100 mL over the duration of the study.

There are times where the protocol requires more than one procedure to be completed at the same time point. In these instances: Scintigraphic imaging takes precedence over other procedures and therefore is collected at the nominal time; Vital signs are recorded after the nominal time; ECG recordings are taken prior to the nominal time; All safety assessments are timed and performed relative to the start of dosing.

Discharge from the Clinical Unit

A subject is allowed to leave the premises following completion of study-specific procedures at 24 h post-dose, providing that:

no AEs have been reported during the study visit

the subject responds in the affirmative when asked if they are feeling well

If any of these conditions are not met, then the subject is only allowed to leave the clinical unit with the authorization of the investigator or appropriately qualified delegate.

Follow-up

A follow-up phone call takes place 3 to 5 days after the final dose to ensure the ongoing wellbeing of the subjects. If a subject reports any AEs which can present a cause for concern, they are required to attend the clinic for a further follow-up assessment (as an unscheduled visit). Completion of the last follow-up call or unscheduled follow-up visit is considered the end of the study.

Dosing of Subjects

Food and Fluid Intake

Meals (breakfast) are required to be controlled by clinical staff members on the first day of each period. Meals are provided at nominal times. A restricted breakfast menu is provided to subjects.

On each of the study days the subjects receive 1 of 3 different breakfasts:

high-fat, high calorie—1000 calories with 50% fat

low-fat, high calorie—1000 calories with 33% fat

low-fat, low-medium calorie—500 calories with 33% fat

The start and stop time of the meal must be recorded in the source workbook and where less than 100% of the meal has been consumed, the percentage and reason behind

<100% consumed must be recorded in the source workbook. Subjects are expected to consume 100% of the meal.

Subjects are allowed water up to 1 h before the scheduled dosing time and are provided with 240 mL of water at 1 h post-dose. Water can be taken as desired except for 1 h before and after IMP administration. Decaffeinated fluids are allowed ad libitum from lunch time on the day of dosing.

Subjects are provided with a light snack and then are too fast from all food and drink (except water) for a minimum of 10 h until the following morning, when they are provided with a breakfast according to the randomization schedule.

The breakfast should be consumed over a maximum period of 25 min, with dosing occurring approximately 30 min after the start of breakfast. Subjects should be encouraged to eat their meal evenly over the 25-min period. It is acknowledged that some subjects take less time to eat, but dosing should still occur approximately 30 min after the start of breakfast. No further food is permitted until 4 h post-dose.

Lunch is provided at approximately 4 h post-dose and an evening meal at approximately 10 h post-dose. On subsequent days, meals are provided at appropriate times. Subjects receive standardized meals scheduled at the same time in each period of the study.

Subjects receive 2000 calories per day. For lunch, all subjects receive approximately 500 calories (approximately 33% fat); for dinner subjects receive the balance of their calories with a target fat content (33% as a percentage of total daily calories). Therefore, subjects receiving the high calorie breakfast would receive a 500-calorie dinner and subjects receiving a low-calorie breakfast would receive a 1000 calorie dinner.

Administration of Test Preparations

Subjects are dosed on the morning of Day 1 of each study period. The exact time of dosing is decided based on logistics. Subjects receive concomitant administration of both IMPs on 3 separate occasions, with a washout of at least 7 days, but no more than 3 weeks, between each administration. 240 mL of water are given immediately following oral administration.

During all clinical phases of the study, subjects are observed by study staff to assure compliance to all study procedures, including dose administration. Compliance of swallowing tablets is confirmed by scintigraphy.

No prescription medication, including hormonal contraceptives and hormone replacement therapy, is permitted from 14 days before Admission until the follow-up call except for the following:

Prescription Topical Medications

Those deemed necessary by the investigator to treat AEs

Not considered to interfere with the objectives of the study as agreed by the PI and sponsor's medical monitor.

No over-the-counter medication or herbal remedies are permitted from the 7 days before Admission except for the following: Single-ingredient, paracetamol products (up to 4 g/day) are permitted; Topical medications are permitted; Not considered to interfere with the objectives of the study as agreed by the PI and sponsor's medical monitor.

Gamma Scintigraphic Assessments

Gamma Scintigraphic Procedures

For Regimens A, B and C, in vivo gamma scintigraphic imaging are performed as follows:

An anterior anatomical marker containing not more than 0.05 MBq (1.35 μCi) 99mTc is taped to the skin where the mid-clavicular line meets with the right costal margin so that it lies in approximately the same transverse plane as the pylorus. A second anatomical marker containing not more than 0.05 MBq (1.35 μCi) 99mTc is taped to the skin posteriorly, directly in line with the anterior marker. All images are acquired with the subjects standing in front of the gamma camera.

Anterior and posterior scintigraphic dual isotope images, each of at least 50 s duration, are recorded using a gamma camera with a 40-cm field of view (FOV) and fitted with a medium energy parallel hole collimator. Image duration is to be increased as necessary to ensure the quality of the data.

Images are recorded immediately after dosing and then at approximately 10 min intervals until 1 h post-dose, every 15 min from 1 h until 4 h post-dose, every 30 min from 4 h until 8 h post-dose. Thereafter, images are acquired at 1 h intervals until 16 h post-dose, and a final image is collected at 24 h post-dose.

The imaging schedule provided is a guide. The actual timing of the acquisition is controlled by the member of staff performing the image; however, images should be recorded ±5 min of the nominal time. Actual image times are recorded.

Typically, a pair of images is required at each time point. However, a second image pair may be required if the entire spread of radioactivity within the GI tract cannot be encompassed by a single FOV. Images may also need to be repeated if, for example, the subject moves during the acquisition or if the position of the camera needs to be adjusted. Adjustments to the schedule may also be made to allow time to alter the fixed marker to maintain image quality. These factors may affect the imaging schedule.

Assessment of Safety

Definition and Classification of Adverse Events

An AE is any untoward medical occurrence in a subject that occurs either before dosing (referred to as a pre-dose AE) or once a medicinal product has been administered, including occurrences which are not necessarily caused by or related to that product. An adverse drug reaction is any AE where a causal relationship with the IMP is at least a reasonable possibility (possibly related or related). Adverse events are monitored from the time the subject signs the ICF until after the final follow-up call. The severity of AEs is assessed as follows:

Mild: An AE that is easily tolerated by the subject, causes minimal discomfort and does not interfere with everyday activities

Moderate: An AE that is sufficiently discomforting to interfere with normal everyday activities; intervention may be needed

Severe: An AE that prevents normal everyday activities; treatment or other intervention usually needed

Assessment of Causality

Every effort should be made by the investigator to try to explain each AE and assess its relationship, if any, to the IMP. The temporal relationship of the event to IMP administration should be considered in the causality assessment (i.e., if the event starts soon after IMP administration and resolves when the IMP is stopped).

Causality should be assessed using the following categories:

Unrelated: Clinical event with an incompatible time relationship to IMP administration, and that could be explained by underlying disease or other drugs or chemicals or is incontrovertibly not related to the IMP

Possibly related: Clinical event with a reasonable time relationship to IMP administration, and that is unlikely to be attributed to concurrent disease or other drugs or chemicals

Related: Clinical event with plausible time relationship to IMP administration and that cannot be explained by concurrent disease or other drugs or chemicals

The degree of certainty with which an AE is attributed to IMP administration (or alternative causes, e.g., natural history of the underlying disease, concomitant therapy, etc.) is determined by how well the experience can be understood in terms of one or more of the following: known pharmacology of the IMP; reactions of a similar nature have been previously observed with the IMP or this class of drug; the experience being related by time to IMP administration, terminating with IMP withdrawal or recurring on re-challenge; alternative cause.

A serious adverse event (SAE) is defined as any untoward medical occurrence that at any dose: results in death; is life-threatening; requires hospitalization or prolongation of existing hospitalization; results in persistent or significant disability or incapacity; consists of a congenital anomaly or birth defect; an important medical event as recognized by the PI SAEs must be immediately reported to the sponsor.

Definition of Suspected Unexpected Serious Adverse Reactions: Suspected unexpected serious adverse reactions (SUSARs) are AEs that are believed to be related to an IMP and are both unexpected and serious.

Laboratory Measurements: Blood and urine samples results are reviewed by a physician and acted upon before the subject is dosed or receives their next dose, or is released from the study, as is appropriate.

Hematology, Clinical Chemistry and Coagulation; Laboratory tests are performed by The Doctors Laboratory. Blood samples are collected in the fasted state and processed.

The acceptable deviations from the nominal blood sampling time points for laboratory assessments are:

The pre-dose blood sample to be taken ≤2 h before dosing; Discharge blood samples to be taken ±1 h from the nominal blood sampling time.

Urinalysis: Urinalysis is performed on-site using a dipstick. If microscopy is required, a urine sample is to The Doctors Laboratory.

The acceptable deviations from the nominal urine sampling time points for urinalysis are:

The pre-dose urine sample is to be taken ≤3 h before dosing or the first void of the day

Discharge urine samples is to be taken ±2 h from the nominal urine sampling time

Pregnancy Test

Serum pregnancy tests at Screening and urine pregnancy tests at Admission are performed.

Follicle-Stimulating Hormone Test

Serum FSH tests are performed.

Drug Screen

A urine drug screen is to be performed on-site using a dipstick method.

Alcohol Breath Test

An alcohol breath test is performed. A positive result excludes the subject from dosing during that admission.

Carbon Monoxide Breath Test

A carbon monoxide breath test is to be performed. Result of greater than 10 ppm excludes the subject from the study.

Abnormal Laboratory Findings

In cases where laboratory findings are outside the normal range and the investigator believes that the results may be of clinical significance, repeat sampling may be requested as clinically indicated. If the abnormal finding is clinically significant, appropriate actions are taken, e.g., the subject is not entered into the study or the subject may be withdrawn from the study. The same applies if the results of the HBsAg, HCV Ab or HIV test are positive and in addition the investigator ensures that adequate counselling is available if requested. Abnormal at follow-up assessments may also require repeat testing if the investigator believes the results may be of clinical significance. Any clinically significant abnormality, including changes from baseline, must be reported as an AE. Additional and/or urine samples may be taken for safety tests. Furthermore, additional assays outside those specified in the protocol may be performed for safety reasons as requested by the investigator.

Vital Signs Measurements

Blood pressure and heart rate are measured by an automated recorder after the subject has been in a supine position for a minimum of 5 min. Table 12. Oral temperature is measured. Table 12. The acceptable deviations from the nominal vital signs measurement time points are:

Discharge vital signs measurements are taken ±1 h from the nominal time point. If a subject shows an abnormal assessment at any stage, repeat measurements may be made and the abnormality followed to resolution if required. Additional measurements may be taken as deemed necessary by the investigator. Any clinically significant abnormality, including changes from baseline, must be reported as an AE.

ECG Measurements

12-lead ECGs are measured after the subject has been in the supine position for a minimum of 5 min as detailed in the study flow chart. The acceptable deviations from the nominal ECG measurement time points are:

Discharge ECG measurements are taken ±1 h from the nominal time point. if a subject shows an abnormal assessment at any stage, repeat measurements may be made and the abnormality followed to resolution if required. Additional measurements may be taken as deemed necessary by the investigator.

Any clinically significant abnormality, including changes from baseline, is to be reported as an AE.

Body Weight

The subject's body weight is measured.

Physical Examination

Subjects undergo a physical examination.

Additional Safety Procedures

Additional non-invasive procedures that are already specified in the protocol may be performed, if it is believed that an important effect of the IMP(s) is occurring or may occur at a time when no measurements are scheduled, or if extra procedures are needed in the interests of safety.

Additional blood samples for safety assessments may be taken if required by the investigator at any point.

Statistics and Data Analysis

Sample Size Justification

The sample size calculation is based on a paired T-test comparing IW-3718 with immediate release colesevelam on complete gastric emptying time T90 after subjects consume a low-fat high calorie breakfast. Fifteen scintigraphic evaluable subjects provide >90% power to detect a difference of 5 hours gastric retention time between IW-3718 with immediate release colesevelam, assuming the standard deviation among different subjects is 5. The type I error is controlled at 0.05 (two-sided).

Scintigraphic Data Analysis

Qualitative and quantitative scintigraphic data analysis are performed to determine the following parameters:

IW-3718

Time for 90% of gastric emptying (T₉₀)

Time for 50% gastric emptying time (T₅₀)

Anatomical location and time of initial tablet disintegration

Anatomical location and time of complete tablet disintegration

Quantitative analysis of tablet erosion

Quantitative analysis of gastric emptying to include eroding tablet remnants

Gastric emptying time of tablet if incomplete disintegration in the stomach

Colon arrival time of tablet if incomplete disintegration before arrival in the colon (if applicable)

Small Bowel transit time (if applicable)

IR colesevelam

Time for 90% of gastric emptying (T₉₀)

Time for 50% gastric emptying time (T₅₀)

Quantitative analysis of gastric emptying of IR colesevelam tablet remnants

Initial and complete gastric emptying time for tablet remnants

Anatomical location and time of initial tablet disintegration

Anatomical location and time of complete tablet disintegration

Analysis of Key Scintigraphic Parameters

A Wilcoxon signed-rank test is used to compare IW-3718 with immediate release colesevelam on complete gastric emptying time (T₉₀) after subjects have consumed a low-fat high calorie breakfast (i.e., Regimen B).

Analysis of Other Scintigraphic Parameters

Wilcoxon signed-rank test is used to compare IW-3718 with immediate release colesevelam on gastric emptying time (T90 and T50) for Regimen A (high-fat, high calorie breakfast) and Regimen C (low-fat, low-medium calorie breakfast), and T50 for Regimen B (low-fat high calorie breakfast). There is no adjustment for multiplicity. P-values are considered nominal for other scintigraphic parameters.

Populations for analysis are determined for safety and scintigraphic data after database lock using the criteria defined in the reporting and analysis plan (RAP); the RAP is to be signed off prior to database lock.

TABLE 9
Investigational Medicinal Products

| | Investigational Medicinal | Dose and Route of |
|---|---|---|
| Treatment | Product | Administration |
| Regimen | ¹¹¹In-IW-3718 Tablet | 1 × 500 mg tablet of |
| A | (500 mg labelled with | ¹¹¹In-labelled |
| | NMT 1 MBq ¹¹¹In) | IW-3718 1 × 625 mg IR ^{99m}Tc- |
| | ^{99m}Tc-colesevelam Tablet | labelled colesevelam tablet |
| | (625 mg labelled with NMT | Oral, 30 minutes after start |
| | 4 MBq ^{99m}Tc) | of high-fat, high calorie |
| | | breakfast |
| Regimen | ¹¹¹In-IW-3718 Tablet | 1 × 500 mg tablet of |
| B | (500 mg labelled with | ¹¹¹In-labelled |
| | NMT 1 MBq ¹¹¹In) | IW-3718 1 × 625 mg IR ^{99m}Tc- |
| | ^{99m}Tc-colesevelam Tablet | labelled colesevelam tablet |
| | (625 mg labelled with NMT | Oral, 30 minutes after start |
| | 4 MBq ^{99m}Tc) | of low-fat, high calorie |
| | | breakfast |
| Regimen | ¹¹¹In-IW-3718 Tablet | 1 × 500 mg tablet of |
| C | (500 mg labelled with | ¹¹¹In-labelled |
| | NMT 1 MBq ¹¹¹In) | IW-3718 1 × 625 mg IR ^{99m}Tc- |
| | ^{99m}Tc-colesevelam Tablet | labelled colesevelam tablet |
| | (625 mg labelled with NMT | Oral, 30 minutes after start of |
| | 4 MBq ^{99m}Tc) | low-fat, low-medium calorie |
| | | breakfast |

Table 10 List of Abbreviations

### Abbreviation Definition

^{99m}Tc technetium-99
¹¹¹In indium-111
AE adverse event
ALT alanine aminotransferase
ARSAC Administration of Radioactive Substances Advisory Committee
CHMP Committee for Medicinal Products for Human Use DGER duodenogastroesophageal reflux
EC ethics committee
ECG electrocardiogram
EMA European Medicines Agency FDA US
Food and Drug Administration FOV field of view
FSH follicle stimulating hormone
GCP good clinical practice
GERD gastroesophageal reflux disease GI gastrointestinal
GP general practitioner HBsAg hepatitis
B surface antigen
HCl hydrochloride
HCV Ab hepatitis C virus antibody
HIV human immunodeficiency virus ICF informed consent form
ICH International Council for Harmonization IMP investigational medicinal product
IR immediate release
ISF Investigator Site File
LDL-c low density lipoprotein-cholesterol MBq megabecquerel
MedDRA Medical Dictionary for Regulatory Activities
MHRA Medicines and Healthcare products Regulatory Agency mSv millisievert
PHE Public Health England
PPI proton pump inhibitor
QA quality assurance
QC quality control
QTc corrected QT
QTcF QT interval corrected for heart rate using Fridericia's formula
RAP Reporting and Analysis Plan SAE serious adverse event
SOP standard operating procedure
SUSAR suspected unexpected serious adverse reaction TEAE treatment-emergent adverse event
WHO DDE World Health Organization Drug Dictionary Enhanced

TABLE 11
CLINICAL LABORATORY PARAMETERS

| | Clinical | | | |
|---|---|---|---|---|
| Hematology | Chemistry | Virology | Urinalysis | Drugs of Abuse |
| Basophils | Alanine | Hepatitis B | Bilirubin | Amphetamines |
| Eosinophils | Aminotransferase | Surface | Blood | Barbiturates |
| Hematocrit | (ALT) | Antigen | Glucose | Benzodiazepines |
| (Packed Cell | Albumin | Hepatitis C | hCG (female subjects | Cocaine |
| Volume- | Alkaline | Antibody | only)^{b} | Marijuana/Cannabis |
| PCV) | phosphatase | HIV | Ketones | Methadone |
| Hemoglobin | aspartate | Antibody | Leukocytes | Methamphetamine/ |
| Lymphocytes | Aminotransferase | | Nitrites | Ecstasy |
| Mean Cell | (AST) | | pH | Morphine/Opiates |
| Hemoglobin | Bicarbonate | | Protein | Phencyclidine |
| (MCH) | Bilirubin (Total) | | Specific | Tricyclic |
| Mean Cell | Bilirubin (Direct) | | gravity | Antidepressants |
| Hemoglobin | (only if total is | | Urobilinogen | |
| Concentration | elevated) | | At discretion of | |
| (MCHC) | Calcium chloride | | investigator based | |
| Mean Cell | Creatine kinase | | on urinalysis | |
| Volume | (CK) creatine | | results | |
| (MCV) | Follicle | | Microbiology | |
| Monocytes | stimulating | | Urine Microscopy | |
| Neutrophils | hormone (FSH; | | | |
| Platelet Count | post-menopausal | | | |
| Red Blood | female subjects | | | |
| Cell (RBC) | at screening | | | |
| Count | only) | | | |
| White Blood | Gamma | | | |
| Cell (WBC) | Glutamyl | | | |
| Count | Transferase | | | |
| Coagulation | (GGT) Glucose | | | |
| Tests | Glucose | | | |
| Prothrombin | (Fasting) | | | |
| Time | hCG (female | | | |
| Activated | subjects only)^{a} | | | |
| partial | Potassium | | | |
| thromboplastin | Phosphate | | | |
| time (APTT) | (Inorganic) | | | |
| | Protein (Total) | | | |
| | Sodium | | | |
| | Urea | | | |

^{a}Screening
^{b}Admission

TABLE 12
STUDY FLOW CHART

| | | Period 1 to 3 | | | | | |
|---|---|---|---|---|---|---|---|
| | | | 1 | | | 2 | Follow Up Call |
| | | | | | Times After | | 3 to 5 |
| | −28 to −2 | −1 | Pre- | | Dosing (h) | | days after |
| Study Day | Screening | Admission | dose | 0 | 1 to 16 | 24 | final dose |
| General | | | | | | | |
| Assessments | | | | | | | |
| Informed Consent | X | | | | | | |
| Inclusion/Exclusion Criteria | X | X | | | | | |
| Medical History | X | X^{a} | | | | | |
| Weight and Height | X | | | | | | |
| Carbon Monoxide | X | X | | | | | |
| Breath Test | | | | | | | |
| Drug Screen | X | X | | | | | |
| Alcohol Breath | X | X | | | | | |
| Test | | | | | | | |
| Serum Pregnancy | X | | | | | | |
| Test | | | | | | | |
| Urine Pregnancy | | X | | | | | |
| Test | | | | | | | |
| FSH | X | | | | | | |
| Virology | X | | | | | | |
| Ability to Swallow | X | | | | | | |
| Empty Size 000 | | | | | | | |
| Capsule | | | | | | | |
| IMP | | | | X | | | |
| Administration | | | | | | | |
| Safety | | | | | | | |
| Assessments | | | | | | | |
| Physical | X | | | | | X^{b} | |
| Examination | | | | | | | |
| Safety Labs^{c} | X | | X^{d} | | | X | |
| Urinalysis | X | | X^{d} | | | X | |
| ECG | X | X | | | | X | |
| Vital Signs^{e} | X | X | | | | X | |

| Adverse Events | [symbol] | |
|---|---|---|
| Scintigraphic | | |
| Assessments | | |
| Dual isotope | | [symbol] |
| anterior and | | |
| posterior images ^{f} | | |

^{a}Medical history update
^{b}Targeted physical examination 24 h after the final dose
^{c}Hematology, coagulation and clinical chemistry
^{d}Admission to Period 1 only
^{e}Blood pressure and heart rate. Oral temperature is measured at screening only
^{f} Images is recorded immediately after dosing and then at approximately 10 min intervals until 1 h post-dose, every 15 min from 1 h until 4 h post-dose, every 30 min from 4 h until 8 h post-dose. Thereafter, images are acquired at 1 h intervals until 16 h post-dose, and a final image is collected at 24 h post-dose.

Results

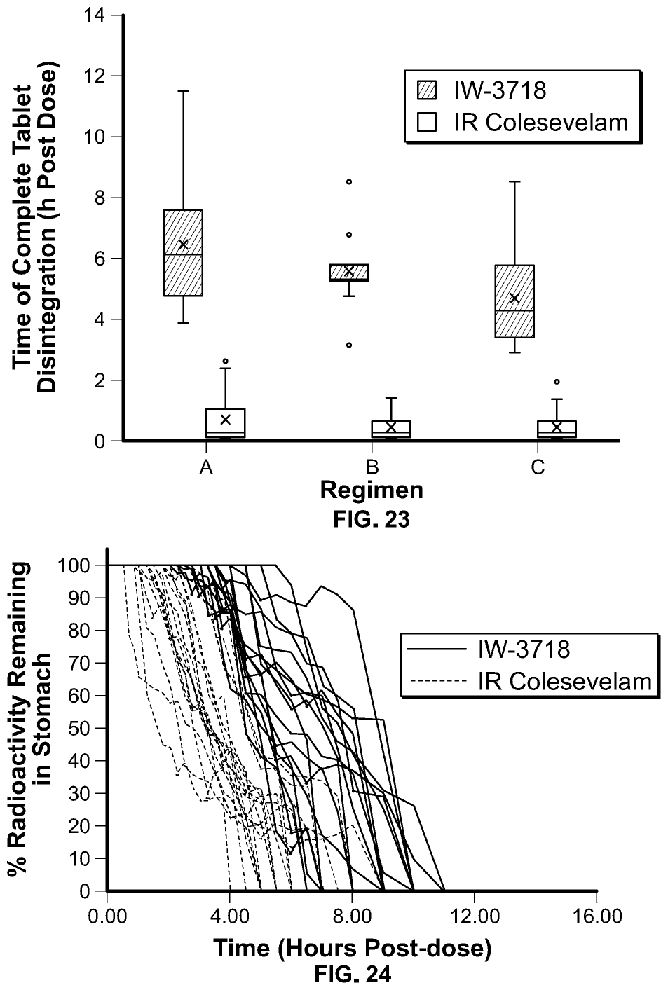

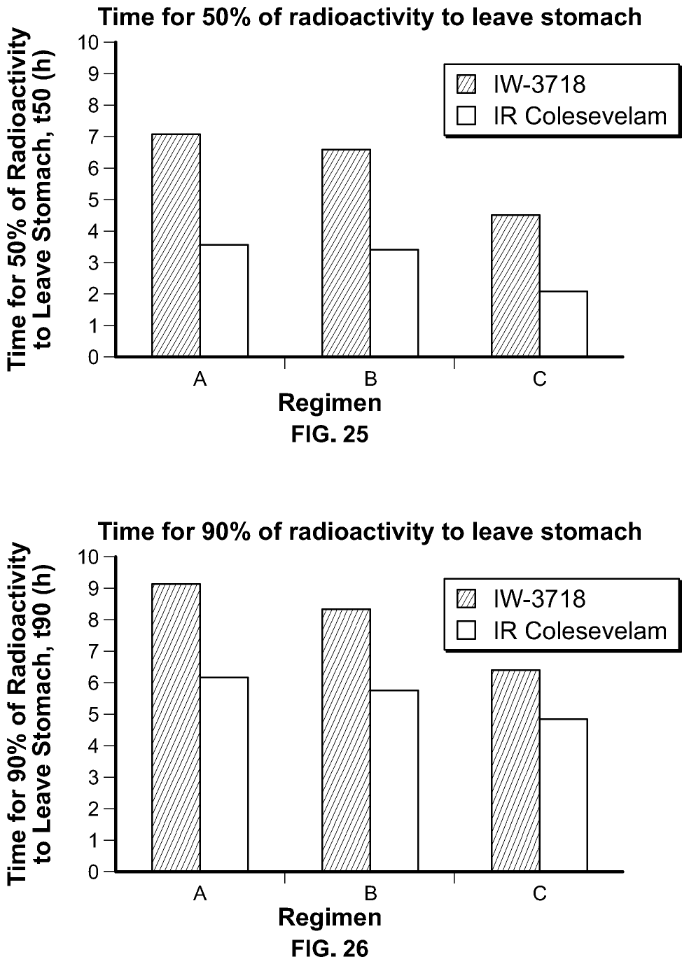

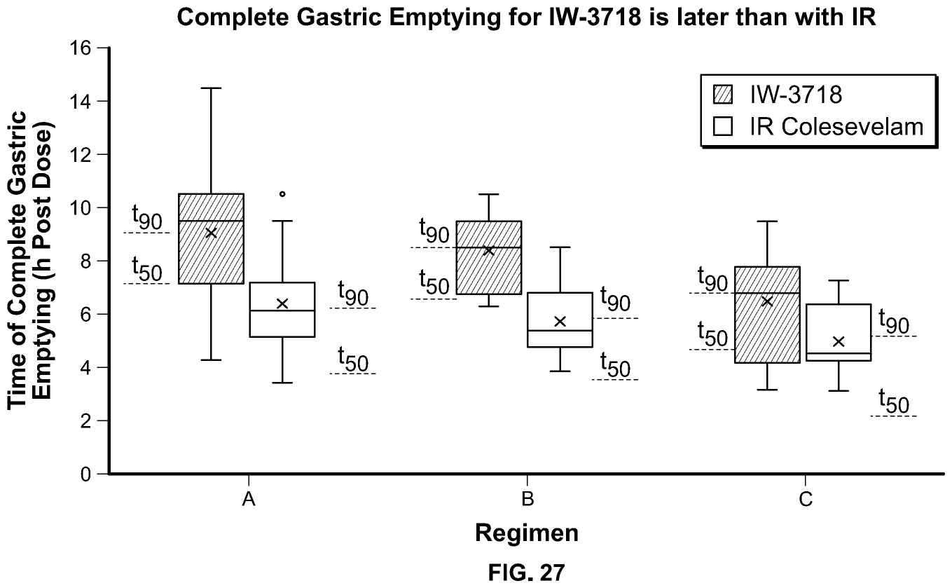

As shown in FIG. 23-FIG. 27, scintigraphy showed that IW-3718, formulated as a gastro-retentive tablet, was retained in the stomach, with mean disintegration in the stomach significantly longer than the immediate release formulation of colesevelam, for subjects in all 3 regimens. The large majority of the IW-3718 tablets (in 16 of 18 subjects) was retained in the stomach until complete disintegration. But not so the IR colesevelam dosage form. FIG. 23. FIG. 24 shows results for each subject and clearly radioactivity from IW-3718 is retained in the stomach longer than seen with the IR dosage form. Also, it took longer for 50% and for 90% of radioactivity from IW-3718 to leave the stomach than that of the IR formulation of colesevelam. FIG. 25 and FIG. 26. Complete gastric emptying of radioactivity from IW-3718 took longer than from the immediate release formulation of colesevelam. 50% of IW-3718 remains in stomach after 90% of IR has emptied with regimens A, B. FIG. 27.

TABLE 13

| | Expected | Actual |
|---|---|---|
| IW-3718 | | |
| Time for complete tablet | 7-8 | 5-6* |
| disintegration (h) | | |
| Residence time of tablet in | 5-6 | 5-6 |
| stomach (h) | | |
| IR colesevelam | | |
| Time for complete tablet | <1 | <1 |
| disintegration (h) | | |
| Residence time of tablet in stomach (h) | n/a | n/a |

*Time for in-vitro disintegration appears to over-estimates disintegration behavior in-vivo

TABLE 13B
Gastric Emptying - Quantitative

| | T50% | T90% |
|---|---|---|
| | (h post-dose) | (h post-dose) |
| IW-3718 | | |
| High fat, high calorie breakfast | 7.069 (1.944) | 9.179 (2.621) |
| [Reg A; n = 18] | | |
| Low fat, high calorie breakfast | 6.589 (1.439) | 8.394 (1.373) |
| [Reg B; n = 18] | | |
| Low fat, low-medium calorie | 4.510 (1.205) | 6.432 (1.946) |
| breakfast | | |
| [Reg C; n = 18] | | |
| Colesevelam | | |
| High fat, high calorie breakfast | 3.567 (1.276) | 6.184 (2.114) |
| [Reg A; n = 18] | | |
| Low fat, high calorie breakfast | 3.399 (0.782) | 5.781 (1.247) |
| [Reg B; n = 18] | | |
| Low fat, low-medium calorie | 2.084 (0.697) | 4.863 (1.313) |
| breakfast | | |
| [Reg C; n = 18] | | |

TABLE 13C
Tablet disintegration

| | Time of | Time of | Total time |
|---|---|---|---|
| | initial tablet | complete tablet | for tablet |
| | disintegration | disintegration | disintegration |
| | (h post-dose) | (h post-dose) | (h) |
| IW-3718 | | | |
| High fat, high | 3.074 (0.918) | 6.441 (2.033) | 3.367 (2.162) |
| calorie breakfast | | | |
| [Reg A; n = 18] | | | |
| Low fat, high | 3.189 (0.940) | 5.550 (1.075) | 2.361 (1.027) |
| calorie breakfast | | | |
| [Reg B; n = 18] | | | |
| Low fat, low-medium | 2.499 (0.645) | 4.659 (1.557) | 2.201 (1.285) |
| calorie breakfast | | | |
| [Reg C; n = 18] | | | |
| Colesevelam | | | |
| High fat, high | 0.038 (0.062) | 0.669 (0.897) | 0.631 (0.874) |
| calorie breakfast | | | |
| [Reg A; n = 18] | | | |
| Low fat, high | 0.084 (0.146) | 0.431 (0.456) | 0.347 (0.368) |
| calorie breakfast | | | |
| [Reg B; n = 18] | | | |
| Low fat low-medium | 0.063 (0.142) | 0.444 (0.507) | 0.381 (0.454) |
| calorie breakfast | | | |
| [Reg C; n = 18] | | | |

See also FIG. 25 to FIG. 27.

TABLE 14
IW-3718 Formulation Used in Examples 1-2

| | Theoretical | Theoretical |
|---|---|---|
| | Quantity per | Quantity per |
| COMPONENT | Tablet (w/w %) | Tablet (mg) |
| Colesevelam Hydrochloride | 46.0 | 500.0 |
| Polyethylene oxide | 46.0 | 500.0 |
| (Polyox WSR N750) | | |
| Butylated hydroxytoluene | 0.06 | 0.65 |
| Colloidal silicon dioxide | 2.0 | 21.7 |
| Magnesium stearate | 0.5 | 5.4 |
| Tablet core weight | 100.0 | 1087 |
| | COATED TABLET | |
| Opadry ® II 85F | 3 | 33 |
| Purified water | NA | NA |
| (removed during processing) | | |
| Microcrystalline cellulose | ~5.4 (adjusted | 59.1 |
| | according to API | |
| | moisture, etc.) | |
| Coated Tablet Weight | NA | 1120 |

Note that the tablet cores and the tablets are about 1100 mg in weight, of which 500 mg is Colesevelam.

TABLE 15
Other Exemplary Dosage Forms

| | Theoretical | Theoretical | Theoretical | Theoretical | Theoretical |
|---|---|---|---|---|---|
| | Quantity | Quantity | Quantity | Quantity | Quantity |
| | per Tablet | per Tablet | per Tablet | per Tablet | per Tablet |
| COMPONENT | (w/w %) | (mg) | (mg) | (mg) | (mg) |
| Colesevelam | 46.0 | 688.97 | 688.97 | 700.0 | 700.0 |
| Hydrochloride | | | | | |
| Polyethylene oxide | 46.0 | 688.97 | 688.97 | 700.0 | 700.0 |
| (Polyox WSR N750) | | | | | |
| Butylated | 0.06 | 0.90 | 0 | 0.90 | 0 |
| hydroxytoluene | | | | | |
| Colloidal silicon | 2.0 | 29.94 | 29.94 | 30.0 | 30.0 |
| dioxide | | | | | |
| Magnesium stearate | 0.5 | 7.45 | 7.45 | 7.5 | 7.5 |
| Microcrystalline | ~5.4(adjusted | 81.55 | 82.45 | 61.6 | 62.5 |
| cellulose | according to | | | | |
| | API moisture, etc.) | | | | |
| Tablet core weight | 100.0 | 1500 | 1500 | 1500 | 1500 |
| | COATED TABLET | | | | |
| Opadry ® II 85F | 3 | 45.54 | 45.54 | 45.0 | 45.0 |

### Summary of Outcomes of Examples 1-2

1500 mg BID dose demonstrated efficacy in HB (heartburn) & RG (regurgitation) with strong safety profile—greater improvement of HB & RG observed for IW-3718 1500 mg vs PBO

1500 mg BID demonstrated >10% more responders vs placebo, using by-patient improvement threshold indicated by PRO analysis as clinically meaningful

The study population is reflective of uGERD (uncontrolled GERD) population, as most patients had positive Bravo (acid reflux); 52% had EE

1500 mg differentiated vs PBO on primary EP (nominal p-value 0.04; LOCF)

Difference was sensitive to analysis method

Magnitude of improvement was consistent for both HB & RG

EE stratum showed modest gains vs overall population

˜12% improvement in responder rate for IW-3718 vs PBO observed in mITT

Scintigraphy shows controlled release of IW-3718 with prolonged gastric-emptying properties differentiated from colesevelam

Quantitative analysis of the mRESQ-eD suggests 44-60% improvement for individual patients is clinically meaningful.

### Example 3. A Randomized, Double-blind, Placebo-controlled, Parallel-group, Dose-range-finding Trial to Determine Safety and Efficacy of a Bile Acid Sequestrant Dosage Form Administered Orally for 8 Weeks to Patients with Gastroesophageal Reflux Disease

Eligibility Criteria

Inclusion Criteria

Each patient must meet all of the following criteria to be eligible for enrollment in this study:

Patient has signed an ICF before any study-specific procedures are performed.

Patient is an ambulatory male or female (if female, nonpregnant) and is at least 18 years old at the Screening Visit.

Patient has a diagnosis of GERD and reports experiencing GERD symptoms (heartburn or regurgitation) on average ≥4 days per week over the last 8 weeks before the Screening Visit.

Patient has been receiving standard-labeled dose, QD, PPI therapy (treatment that, according to the Investigator's judgment, could not be further improved by changing the brand or timing of PPI administration) for a minimum of 8 weeks before the Screening Visit. Patients should be on a PPI dose and schedule that is consistent with the approved labeling. Patients who have their PPI modified during the Screening Period may be re-screened after 8 weeks of standard-labeled dose, QD, PPI therapy provided they have not previously entered the Pretreatment Period.

Up to 96 hours of pH monitoring (with a Bravo® device) during the Screening Period (while the patient continues taking their PPI) demonstrates evidence of pathological acid reflux (pH is <4 for ≥4.2% of the recording time) during at least 1 of the 24-hour time intervals of pH testing with the Bravo® device as confirmed by centralized review of pH monitoring.

During the last 7 days before randomization patient reports an average heartburn severity (HS, maximum of Items #1 and #2 on mRESQ-eD) of ≥2 (mild) and has a daily HS≥3 (moderate) for at least 2 of those days.

Female patients must be postmenopausal for ≥1 year, surgically sterile (i.e., bilateral oophorectomy, hysterectomy, or tubal sterilization [tie, clip, band, or burn]); or must agree to completely abstain from heterosexual intercourse; or, if heterosexually active, must agree to use 1 of the following methods of birth control from the date she signs the ICF until 24 hours after their final dose of study drug:

a. Progesterone implant or an intrauterine device (IUD)

b. Combination of 2 highly effective birth control methods (e.g., diaphragm with spermicide plus a condom, condom with spermicide plus a diaphragm or cervical cap, hormonal contraceptive [e.g., oral and transdermal patch] plus a barrier method, partner with vasectomy [conducted>60 days before the Screening Visit or confirmed via sperm analysis] plus a hormone or barrier method).

Females of childbearing potential must have a negative urine or serum pregnancy test at the Screening Visit and at the Randomization Visit prior to dosing. Positive urine test results will be confirmed by a serum pregnancy test.

Patient agrees not to make any changes to their usual diet or exercise regimen during the study.

Patient has adequately completed the eDiary questions on at least 5 days each week during the 14 days before the start of the Treatment Period and has completed the weekly questions at least once during the 7 days prior to randomization.

Patient is compliant with QD PPI dosing during the 14 days before the start of the Treatment Period. Patients are considered compliant if, as reported in the eDiary, they take their PPI on at least 5 days each week.

Patient is fluent and literate in at least one of the languages to be used for PRO assessments.

Patient is able to operate the eDiary adequately and agrees to adhere to the study requirements.

For patients who are receiving supplementation of a fat-soluble vitamin to correct or avoid a fat-soluble vitamin deficiency, the patient is willing to take the vitamin supplement at least 4 hours before taking study medication.

Exclusion Criteria

Patients who meet any of the following criteria is not eligible to participate in the study:

Patient has a history of complete lack of GERD symptom response to PPIs.

Patient reports epigastric pain or epigastric burning as his or her predominant symptom at the Screening Visit.

Patient has a diagnosis of gastroparesis per gastric emptying study, or a history of bowel obstruction, or is at risk for a bowel obstruction (e.g., patient has an organic gastrointestinal [GI] motility disorders or a history of major GI surgery).

3. Patient has a history of serum triglyceride concentrations>500 mg/dL on a fasting specimen, or has serum triglyceride concentrations>500 mg/dL on a fasting specimen at Screening or any time during the Pretreatment Period.

Patient has a history of hypertriglyceridemia-induced pancreatitis.

In the Investigator's opinion, patient is susceptible to a deficiency of fat-soluble vitamins (especially vitamin D deficiency; e.g., the patient has osteoporosis or osteomalacia) and is put at risk by receiving colesevelam for 8 weeks.

Patient has an active swallowing disorder that would compromise their ability to swallow the study medication.

Patient has any alarm symptoms including, but not limited to, GI bleeding, anemia, vomiting, or unexpected weight loss at any time during the Screening or Pretreatment Periods.

Patient has undergone surgery that meets any of the following criteria:

a. Surgery of the GI tract (including gastric banding) other than an appendectomy, cholecystectomy, or minor oral or rectal surgery (e.g., tonsillectomy, hemorrhoidectomy, rectocele repair) at any time before the Screening Visit.

b. An appendectomy during the 3 months before the Screening Visit or a cholecystectomy during the 6 months before the Screening Visit or minor oral or rectal surgery during the 30 days before the Screening Visit.

c. Non-GI surgery of the abdomen, pelvis, or retroperitoneal structures during the 6 months before the Screening Visit.

d. Thoracic surgery during the 6 months before the Screening Visit.

e. Other major non-GI surgery during the 30 days before the Screening Visit.

Patient has previously undergone thoracic or abdominal radiotherapy.

Patient has large (>5 cm) hiatal hernia.

EGD, conducted during the Screening Period, reveals that the patient has long-segment Barrett's esophagus (greater than 3 centimeters) or definite dysplastic changes in the esophagus, peptic ulcer disease, active GI bleeding, presence of symptomatic esophageal strictures, presence of esophageal or fundic varices, erosive gastritis, or eosinophilic, herpetic or Candida esophagitis.

Patient has Gilbert's disease, Crohn's disease, diabetes mellitus (defined as A1C>6.5%), Zollinger-Ellison syndrome, pancreatitis, cholecystitis, or systemic sclerosis.

Patient has elevated (defined as >1.5 times the upper limit of normal by the laboratory) levels of serum bilirubin at Screening or any time during the Pretreatment Period.

14. Patient has a history of clinically significant hypersensitivity or allergies to any of the excipients contained in the study medication (active or placebo).

Patient has a history of cancer (resected basal cell or squamous cell carcinoma is acceptable). Note: patients with a history of cancer are allowed provided that the malignancy has been in complete remission for at least 5 years before the Screening Visit. A complete remission is defined as the disappearance of all signs of cancer in response to treatment.

Patient has active substance abuse or history of chronic substance abuse (including alcoholism but not including nicotine) within 12 months before the Screening Visit or is positive for any of the following at the Screening Visit unless legally prescribed for anything but gastrointestinal pain: amphetamines, benzodiazepines, opiates, barbiturates, cocaine, or phencyclidine.

Marijuana use, whether prescribed or not, is prohibited from 30 days before screening through the duration of the study. Use of illicit drugs is not allowed during the study.

Patient has any clinically significant finding on a physical exam, 12-lead electrocardiogram (ECG), or clinical laboratory test after signing the ICF but before receiving the first dose of study medication. (Note: The Investigator will determine if a particular finding is clinically significant. In making this determination, the Investigator will consider whether the particular finding could prevent the patient from performing any of the protocol-specified assessments, could represent a condition that would exclude the patient from the study, could represent a safety concern if the patient participates in the study, or could confound the study-specified assessments of safety or efficacy.)

Patient reports using a prohibited medication during the Screening or Pretreatment Periods, or is not willing or able to abide by the restrictions regarding use of prohibited medications.

19. Patient has received an investigational drug during the 30 days before the Screening Visit, or is planning to receive another investigational drug or use an investigational device at any time during the study.

Patient has an acute or chronic condition that, in the Investigator's opinion, would limit the patient's ability to complete or participate in this clinical study.

Patient has previously entered the Treatment Period of a IW-3718 study.

22. Patient has previously entered the Pretreatment Period of this study. (Note: patients who failed the Pretreatment Period due to abnormal laboratory findings or timing issues may be re-screened.)

23. Patient is enrolled in this study at another clinical study site; is an employee of the Institution or Ironwood Pharmaceuticals; or is a first-degree family member, significant other, or relative residing with an employee of the Institution or Ironwood Pharmaceuticals.

### Example 4 Comparative Data of Two Enteric-coated Oral Dosage Forms in the Form of a Tablet of a Bile Acid Sequestrant Dispersed in a Polymeric Matrix

TABLE 16
Composition of the Two Formulations

| Formulation | 2a | 2b |
|---|---|---|
| API (Colesevelam Hydrochloride) | | 46 |
| DSM | | |
| API (Colesevelam Hydrochloride) | 50 | |
| Formosa | | |
| Polyethylene oxide (Polyox ™ | | 46 |
| WSR N-750) | | |
| Polyethylene oxide (Polyox ™ | 25 | |
| WSR N-60K) | | |
| Colloidal silicon dioxide | | 2 |
| (Aerosil 200) | | |
| Butylated hydroxytoluene | | 0.06 |
| Microcrystalline Cellulose | 24.5 | 5.44 |
| Magnesium Stearate | 0.5 | 0.5 |
| Pharmaceutical coat (3%) | HPMC based | PVA based |
| | (hypromellose and | (Opadry ® |
| | diacetylated | II 85F) |
| | monoglycerides) | |

Formulation 2b comprise PEG-7M (polyethylene oxide CAS Number 25322-68-3, approximate molecular weight 300,000 (Polyox™ WSR N-750)), which is a lower molecular weight polyethylene oxide than Polyox™ WSR N-60K (INCI name: PEG-45M) in formulation 2a. Formulation 2b has an increased amount of polyethylene oxide (46%) than 2a (25%). Formulation 2b comprises BHT as an anti-oxidant, to inhibit oxidative cleavage; formulation 2a does not comprise BHT. Formulation 2b comprises colloidal silicon dioxide and 2a does not. The source of the API has changed from Formosa to DSM.

Formulation 2a tablets were packaged in 100 cc, white opaque, induction sealed high density polyethylene (HDPE) bottles with two 2 g silica gel desiccant sachets. Each bottle contained thirty-five (35) tablets and is fitted with a polypropylene child resistant (CR) cap. Drug Product was stored refrigerated.

Formulation 2b tablets are packaged in Aclar blister strips. Each strip contains 6 tablets. Drug Product is stored refrigerated.

Stability

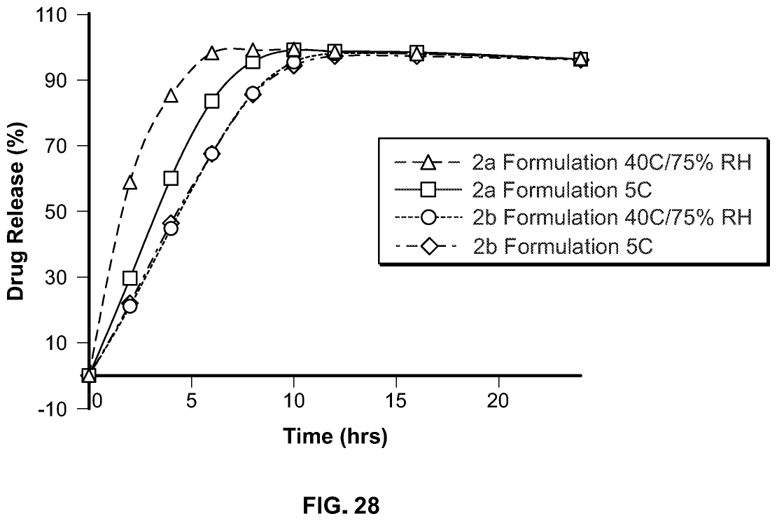

Formulation 2b, an embodiment of the disclosed dosage form, is more stable than formulation 2a. FIG. 28-FIG. 30 show that drug release over time data for formulation 2a and 2b.

Formulation 2a releases drug more quickly at after 1 week in storage at 5° C. than after 1 week in storage at 5° C.; and even more quickly after 1 week in storage at 50° C. In contrast, formulation 2b has the same drug release profile in all 3 tested conditions (after 2 weeks in storage at 5° C.; after 3 months in storage at 40° C./75% RH; and after 4.5 months in storage at 40° C./75% RH). FIG. 28.

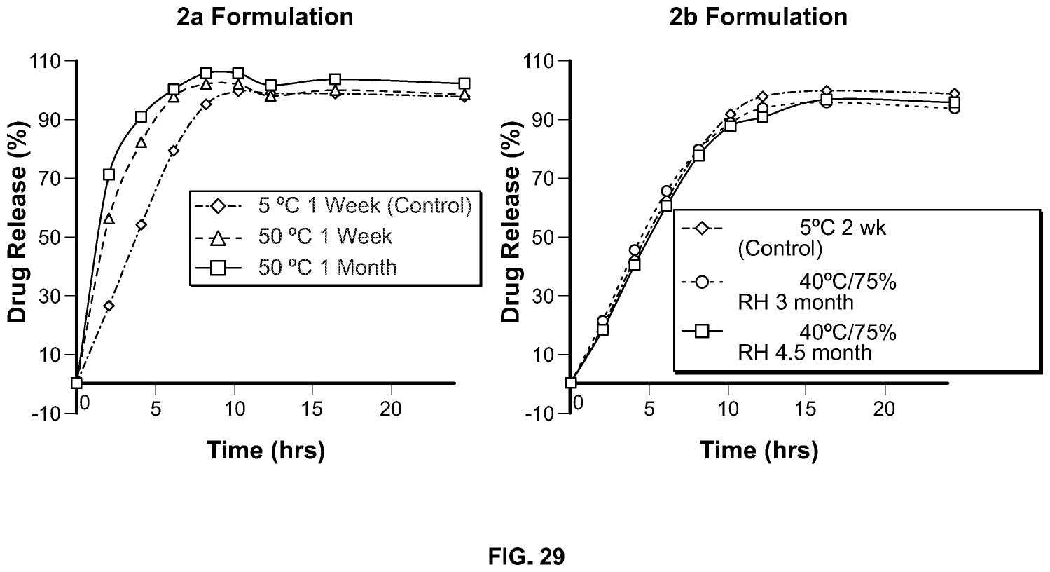

Formulation 2a releases drug more quickly after storage at 40° C./75RH for one month than at 5° C. for one month; formulation 2b releases drug at the about the same rate after storage at 40° C./75RH for one month than at 5° C. for one month. FIG. 29 and Table 17.

TABLE 17

| | Time (hour) | | | | | | | | |
|---|---|---|---|---|---|---|---|---|---|
| | 0 | 2 | 4 | 6 | 8 | 10 | 12 | 16 | 24 |
| 2a Formulation 40° C./ | 0 | 59 | 85 | 98 | 99 | 99 | 98 | 98 | 96 |
| 75% RH | | | | | | | | | |
| 2a Formulation 5° C. | 0 | 30 | 60 | 83 | 95 | 99 | 99 | 98 | 96 |
| 2b Formulation 40° C./ | 0 | 21 | 45 | 67 | 86 | 95 | 98 | 98 | 96 |
| 75% RH | | | | | | | | | |
| 2b Formulation 5° C. | 0 | 22 | 46 | 67 | 85 | 94 | 97 | 97 | 96 |

API Source

Two identical formulations, except for the API, were prepared; 1 from Formosa and 1 with DSM. Table 18 shows that the formulation with the DSM API (IW-ELN-000433-04A) disintegrated more slowly than the one with Formosa (IW-ELN-000433-041).

TABLE 18
pH 4.5 Acetate Buffer

| | | Disintegration Time |
|---|---|---|
| Batch | Tablet | (hours:minutes) |
| IW-ELN-000433-04a | 1 | 16:13 |
| (DSM Drug Substance) | 2 | 14:29 |
| | 3 | 14:38 |
| IW-ELN-000433-04I | 1 | 9:22 |
| (Formosa Drug Substance) | 2 | 8:30 |
| | 3 | 9:30 |

Production Method (Direct Compression Vs Dry Granulation)

Formulation 2a utilized a dry granulation process to aid flowability and compression. The drug product was manufactured with MCC KG1000 to achieve the highest compression but that MCC is not the best for flow (powder properties of KG1000 are designed for compressibility not flow). Formulation 2b was made by direct compression process was chosen. Direct compression may not have been 100% unachievable for formulation 2a but initial work indicated it was not. Direct compression is beneficial because it removes a unit operation saving time and money for every single batch. At a 1200 kg scale it is at least one day of work/batch.

BHT

Butylated hydroxytoluene (BHT) is generally recognized as safe (GRAS) for use in food when the total content of antioxidants is not over 0.02 percent of fat or oil content, including essential (volatile) oil content of food, provided the substance is used in accordance with good manufacturing practice. For a reference value of 70 g daily fat for an average adult, 0.02%=14 mg BHT. This is very much in line with the European Food Safety Authority 2012 recommendation of 0.25 mg/kg/day BHT, which for a 60 kg patient=15 mg BHT. Current formulation of IW-3718 tablets contains ˜4.8 mg in the targeted maximum dose of 8 tablets (4000 mg IW-3718). The level of BHT in IW-3718 is below the level recognized as safe.

Four formulations were prepared at 0% (no added BHT), 0.06% (added), 0.12% (added) and 0.2% (added) BHT using DSM API. These 4 samples were tested after storing for 1 week at 50° C., 1 month at 50° C., and 1 month at 40° C./75% RH. After 1 week no difference was shown between formulations with 0.06% and 0.12% BHT so only 0.06% was tested. 0.12 and 0.2% were pulled at future time points but never tested.

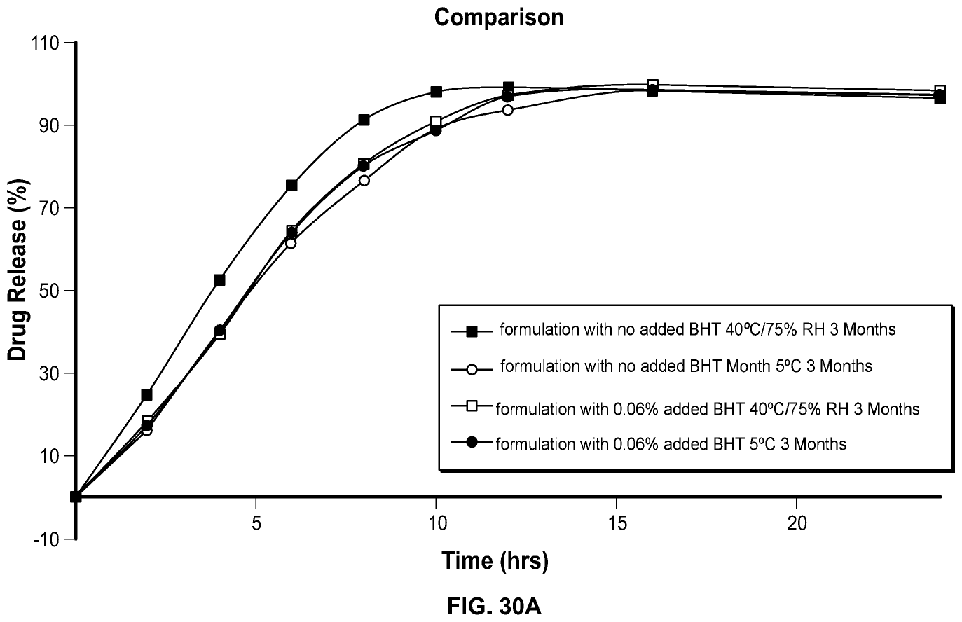

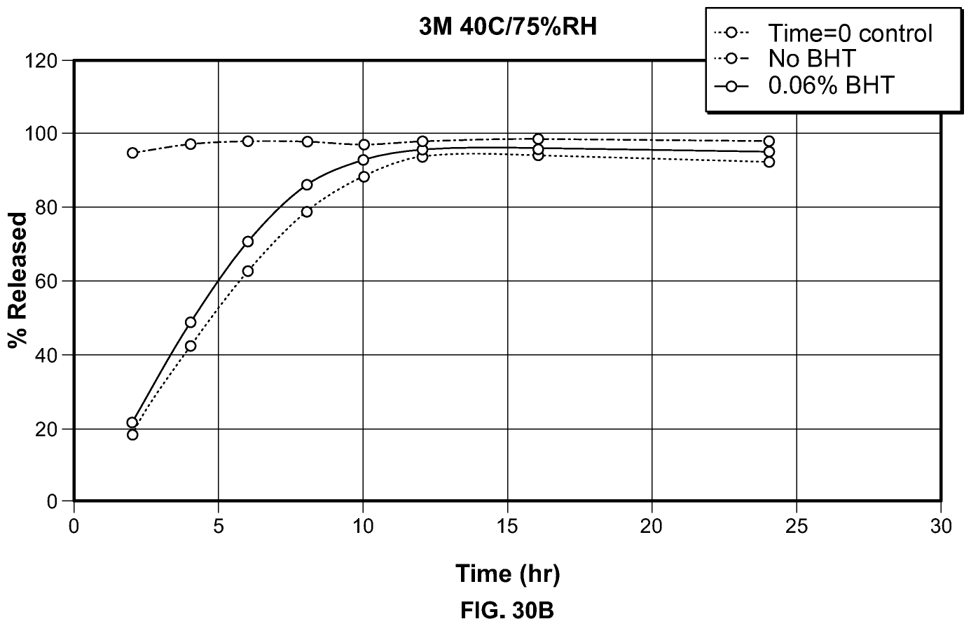

A formulation with 0.06% added BHT after 3 months storage at 40° C./75% RH is as stable as the same formulation stored at 5° C. for 3 months. FIG. 30a. A formulation without 0.06% added BHT after storing at 40° C./75% RH starts to be less stable compared to the same formulation stored at 5° C. for 3 months. FIG. 30a. Primera forced PEO degradation study, FIG. 30b, shows the contribution of 0.06% BHT to formulation stability.

### OTHER EMBODIMENTS

The foregoing description discloses only exemplary embodiments of the invention.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes occur to those skilled in the art. It is therefore to be understood that the appended claims are intended to cover all such modifications and changes as fall within the true spirit of the invention.

What is claimed is:

1. A method of reducing one or more symptoms of gastroesophageal reflux disease (GERD) in a human patient with symptomatic GERD not completely responsive to proton pump inhibitors (PPIs), comprising administering to the patient a therapeutically effective amount of an enteric coated gastro-retentive oral dosage form in the form of a tablet of a bile acid sequestrant dispersed in a polymeric matrix consisting essentially of polyethylene oxide CAS Number 25322-68-3, approximate molecular weight 300,000 (PEG-7M) and one or more filler or compressing agent selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate, wherein the tablet has a tablet core and is coated with an enteric coating, for prolonged retention of the bile acid sequestrant to the stomach of the patient, and administering a pharmaceutical composition comprising a PPI;
wherein the patient experiences a clinically meaningful reduction in one or more symptoms of GERD.

2. The method of claim 1, wherein the bile acid sequestrant is colesevelam or colesevelam hydrochloride.

3. The method of any one of the preceding claims, wherein the patient is administered a dose of 500 mg, 700 mg, 750 mg, 1,000 mg, 1400 mg, 1,500 mg, or 2,100 mg, or more, of the bile acid sequestrant, twice per day.

4. The method of any one of the preceding claims, wherein the patient is administered a dose of 1,500 mg of the bile acid sequestrant, twice per day.

5. The method of any one of the preceding claims, wherein the dose of 1,500 mg is administered as either 2 tablets, each tablet having 750 mg of the bile acid sequestrant or as 3 tablets, each tablet having 500 mg of the bile acid sequestrant, twice per day.

6. The method of any one of the preceding claims, wherein the dose of 1,500 mg is administered as 2 tablets, each tablet having 750 mg of the bile acid sequestrant.

7. The method of any one of the preceding claims, wherein the dose of 1,500 mg is administered as 3 tablets, each tablet having 500 mg of the bile acid sequestrant, twice per day.

8. The method of any one of the preceding claims, wherein prior to administering said enteric coated gastro-retentive, oral dosage form in the form of a tablet of a bile acid sequestrant, the patient was not completely responsive to other treatments, including individually optimized, standard-labeled dose daily PPI therapy for a minimum of 8 weeks.

9. The method of any one of the preceding claims, wherein the patient has erosive esophagitis.

10. The method of claim 9, wherein the patient has erosive esophagitis on esophagogastroduodenoscopy (EGD) with approximately 48 to 96 hours of pH monitoring with a catheter-free, capsule-based pH monitoring device that is attached to the patient's esophagus.

11. The method of claim 9 or claim 10, wherein the patient has evidence of pathological acid reflux on EGD with approximately 48 to 96 hours of pH monitoring with a catheter-free, capsule-based pH monitoring device that is attached to the patient's esophagus.

12. The method of any one of claims 1-11, wherein said enteric coated gastro-retentive, oral dosage form in the form of a tablet of a bile acid sequestrant is administration for eight weeks (eight treatment weeks) or more.

13. The method of any one of the preceding claims, wherein the patient experiences a clinically meaningful weekly heart burn severity score reduction compared to baseline.

14. The method of any one of the preceding claims, wherein the patient experiences a clinically meaningful weekly heart burn severity score reduction compared to baseline of at least 30% for at least four of the eight treatment weeks, including at least one of the last two weeks.

15. The method of any one of the preceding claims, wherein the patient experiences a clinically meaningful weekly heart burn severity score reduction compared to baseline of at least 45% for at least four of the eight treatment weeks, including at least one of the last two weeks.

16. The method of any one of the preceding claims, wherein the patient experiences a clinically meaningful Weekly Regurgitation Frequency Score (WRFS) reduction compared to baseline.

17. The method of any one of the preceding claims, wherein the patient experiences a clinically meaningful a clinically meaningful Weekly Regurgitation Frequency Score (WRFS) reduction compared to baseline of at least 30% for at least four of the eight treatment weeks, including one of the last two weeks.

18. The method of any one of the preceding claims, wherein the patient experiences a clinically meaningful a clinically meaningful Weekly Regurgitation Frequency Score (WRFS) reduction compared to baseline of at least 45% for at least four of the eight treatment weeks, including one of the last two weeks.

19. The method of any one of the preceding claims, wherein the dosage form is retained in the stomach until it is substantially or completely disintegrated.

20. The method of any one of the preceding claims, wherein the enteric coated gastro-retentive, oral dosage form further comprise at least about 0.06% per weight butylated hydroxytoluene of the tablet core.

21. A method of reducing one or more symptoms of gastroesophageal reflux disease (GERD) in a human patient with symptomatic GERD not completely responsive to proton pump inhibitors (PPI), comprising administering a therapeutically effective amount of an enteric coated gastro-retentive oral dosage form in the form of a tablet of colesevelam or colesevelam hydrochloride a dispersed in a polymeric matrix consisting essentially of polyethylene oxide CAS Number 25322-68-3, approximate molecular weight 300,000 (PEG-7M) and one or more filler or compressing agent selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate, wherein the tablet has a tablet core and is coated with a polyvinyl alcohol based enteric coating, for prolonged retention of the bile acid sequestrant in the stomach of the patient in a dose of 1,500 mg twice daily;
wherein: wherein prior to administering said enteric coated gastro-retentive, oral dosage form in the form of a tablet of a bile acid sequestrant, the patient was not completely responsive to other treatments, including individually optimized, standard-labeled dose daily PPI therapy for a minimum of 8 weeks, the patient has erosive esophagitis; said enteric coated gastro-retentive, oral dosage form in the form of a tablet of a bile acid sequestrant is administration for eight weeks (eight treatment weeks);
the dosage form is retained in the stomach until it is substantially or completely disintegrated;
the patient experiences a clinically meaningful weekly heart burn severity score reduction compared to baseline; and clinically meaningful Weekly Regurgitation Frequency Score (WRFS) reduction compared to baseline.

22. The method of claim 21, wherein the patient experiences a clinically meaningful weekly heart burn severity score reduction compared to baseline of at least 30% for at least four of the eight treatment weeks, including at least one of the last two weeks.

23. The method of claim 21 or claim 22, wherein the patient experiences a clinically meaningful weekly heart burn severity score reduction compared to baseline of at least 45% for at least four of the eight treatment weeks, including at least one of the last two weeks.

24. The method of any one of claims 21 to 23, wherein the patient experiences a clinically meaningful Weekly Regurgitation Frequency Score (WRFS) reduction compared to baseline of at least 30% for at least four of the eight treatment weeks, including at least one of the last two weeks.

25. The method of any one of claims 21-24, wherein the patient experiences a clinically meaningful Weekly Regurgitation Frequency Score (WRFS) reduction compared to baseline of at least 45% for at least four of the eight treatment weeks, including at least one of the last two weeks.

26. The method of any one of claims 21-25, wherein the enteric coated gastro-retentive, oral dosage form further comprise at least about 0.06% butylated hydroxytoluene by weight of the tablet core.

27. An enteric coated gastro-retentive oral dosage form in the form of a tablet comprising: colesevelam or colesevelam hydrochloride dispersed in a polymeric matrix consisting essentially of PEG-7M (polyethylene oxide CAS Number 25322-68-3, approximate molecular weight 300,000 (Polyox™ WSR N-750)) and one or more filler or compressing agent selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate, wherein the tablet has a tablet core and is coated with an enteric coating, for prolonged retention of the bile acid sequestrant in the stomach.

28. The dosage form of claim 27, wherein the dosage form is for prolonged retention in the stomach until it is substantially or completely disintegrated.

29. The dosage form of claim 27 or claim 28, wherein the one or more filler or compressing agent is microcrystalline cellulose at 1-10% w/w of the tablet core, butylated hydroxytoluene at about 0.01 to about 0.10% w/w of the tablet core, colloidal silicon dioxide at about 1-5% w/w of the tablet core, magnesium stearate at about 0.1 to 1.0% w/w of the tablet core.

30. The dosage form of any one of claims 27 to 29, wherein the enteric coating is a polyvinyl alcohol based enteric coating.

31. The dosage form of claim 30, wherein the enteric coating is a polyvinyl alcohol based enteric coating.

32. The dosage form of claim 31, wherein the enteric coating is a polyvinyl alcohol based enteric coating at about 1-5% w/w of the tablet core.

33. The dosage form of any one of claims 27 to 32, wherein the PEG-7M (polyethylene oxide CAS Number 25322-68-3, approximate molecular weight 300,000 (Polyox™ WSR N-750)) is about 30 to about 60% w/w of the tablet core.

34. The dosage form of any one of claims 27-33, wherein the PEG-7M (polyethylene oxide CAS Number 25322-68-3, approximate molecular weight 300,000 (Polyox™ WSR N-750)) is about 46% w/w of the tablet core.

35. The dosage form of any one of claims 27-34, wherein the enteric coating is a polyvinyl alcohol based enteric coating at about 3% w/w of the tablet core.

36. The dosage form of any one of claims 27-35, wherein the one or more filler or compressing agent is microcrystalline cellulose at about 5.4% w/w of the tablet core, butylated hydroxytoluene at about 0.06 w/w of the tablet core, colloidal silicon dioxide at about 2.0% w/w of the tablet core, magnesium stearate at about 0.5% w/w of the tablet core.

37. The dosage form of any one of claims 27-36, wherein the enteric coated gastro-retentive, oral dosage form further comprise at least about 0.06% butylated hydroxytoluene per weight of the tablet core.

38. A pharmaceutical composition comprising the gastro-retentive oral dosage form of claim 27.

39. The pharmaceutical composition of claim 38, further comprising an additional therapeutic agent.

40. A method of treating a disease selected from heartburn, indigestion, dyspepsia, erosive esophagitis, peptic ulcer, gastric ulcer, esophageal ulcers, esophagitis, laryngitis, pharyngitis, coarse voice, gastroesophageal reflux disease (GERD), Barrett's esophagus, gastric cancer, esophageal cancer (e.g., adenocarcinoma), gastritis and GERD-related pulmonary dysfunction, and symptomatic GERD not completely responsive to proton pump inhibitor, comprising administering a therapeutically effective amount of a gastro-retentive, oral dosage form of claim 27 or the pharmaceutical composition of claim 39 to a subject in need thereof.

41. The method of claim 40, wherein the disease is symptomatic GERD not completely responsive to proton pump inhibitor.

42. A method to treat/prevent signs and/or symptoms associated with bile acid reflux comprising administering to the patient a therapeutically effective amount of an enteric coated gastro-retentive oral dosage form in the form of a tablet of a bile acid sequestrant dispersed in a polymeric matrix consisting essentially of poly(alkylene)oxide and one or more filler or compressing agent selected from microcrystalline cellulose, butylated hydroxytoluene, colloidal silicon dioxide, lactose, starch, maltodextrins, magnesium stearate, diacetylated monoglycerides, hypromellose, and dibasic calcium phosphate, wherein the tablet is coated with an enteric coating, for prolonged retention of the bile acid sequestrant to the stomach of the patient, to a patient in an amount effective to ameliorate, reduce, palliate, lessen, delay, and/or alleviate one or more of the signs and/or symptoms associated with bile acid reflux.

43. The method of claim 42, wherein the bile acid sequestrant is colesevelam or colesevelam hydrochloride.

44. The method of claim 42 or claim 43, wherein the dosage form is retained in the stomach until it is substantially or completely disintegrated.
